SEQUENCE LISTING (96 sequences)

SEQ ID NO: 1 - DNA, 20 bp, Artificial Sequence
  Primer
  SpCas9 Reverse
aaaagcaccgactcggtgcc

SEQ ID NO: 2 - DNA, 65 bp, Artificial Sequence
  Primer
  SpCas9 Forward
  misc_feature (26)..(45): N = Any nucleotide
gcgcgctaatacgactcactataggnnnnnnnnnnnnnnnnnnnngttttagagctagaaatagc

SEQ ID NO: 3 - DNA, 20 bp, Artificial Sequence
  Protospacer
  Outer cut
gtgcttcgatatcgatcgtt

SEQ ID NO: 4 - DNA, 23 bp, Artificial Sequence
  Primer
  pJ327
agcctagtccaagttttagagag

SEQ ID NO: 5 - DNA, 24 bp, Artificial Sequence
  Primer
  pJ328
aaactgcccagaacaccaaactgg

SEQ ID NO: 6 - DNA, 18 bp, Artificial Sequence
  Primer
  M13 Forward
tgtaaaacgacggccagt

SEQ ID NO: 7 - DNA, 17 bp, Artificial Sequence
  Primer
  M13 Reverse
caggaaacagctatgac

SEQ ID NO: 8 - DNA, 25 bp, Artificial Sequence
  Primer
  T7 Promoter
gcgcgctaatacgactcactatagg

SEQ ID NO: 9 - DNA, 20 bp, Artificial Sequence
  Primer
  pJ161
aaaagcaccgactcggtgcc

SEQ ID NO: 10 - DNA, 46 bp, Artificial Sequence
  Primer
  pJ227
gcgcgctaatacgactcactataggtaatttctactaagtgtagat

SEQ ID NO: 11 - DNA, 20 bp, Artificial Sequence
  Primer
  pJ335
gaatctcccttgctaaccat

SEQ ID NO: 12 - DNA, 20 bp, Artificial Sequence
  Primer
  pJ338
atgttgcatctataaaagaa

SEQ ID NO: 13 - DNA, 23 bp, Artificial Sequence
  Primer
  pJ343
ggtttgattgcatgtgataaccc

SEQ ID NO: 14 - DNA, 23 bp, Artificial Sequence
  Primer
  pJ348
aaagaaatataatgttgcatcta

SEQ ID NO: 15 - DNA, 20 bp, Artificial Sequence
  sgRNA
  Exon 20 Targeting
gtgcttcgatatcgatcgtt

SEQ ID NO: 16 - DNA, 80 bp, Artificial Sequence
  Primer
  pJ356
gaaaaaggaaaatgcttccatatgtatgttaaagaatctcccttgctaaccatttttactgaatgaaggaatggctcctg

SEQ ID NO: 17 - DNA, 85 bp, Artificial Sequence
  Primer
  pJ358
cttaacaaatacagcattacttgagacaaaagaaatataatgttgcatctataaaagaatttatgggacggatttgctattttac

SEQ ID NO: 18 - DNA, 93 bp, Artificial Sequence
  Primer
  pJ357
gtgcttcgatatcgatcgtttgggaaaggaccaacttgtaatgttggtttgattgcatgtgataaccctaaaagaaaaaggaaaatgcttcca

SEQ ID NO: 19 - DNA, 87 bp, Artificial Sequence
  Primer
  pJ359
gtgcttcgatatcgatcgtttggcatatagaaattagcattaaactgcccagaacaccaaactggaccttaacaaatacagcattac

SEQ ID NO: 20 - DNA, 24 bp, Artificial Sequence
  Primer
  pJ375
cgatcagcatactcactgcttcag

SEQ ID NO: 21 - DNA, 24 bp, Artificial Sequence
  Primer
  pJ361
caggaggccttccatctgttgctg

SEQ ID NO: 22 - DNA, 23 bp, Artificial Sequence
  Primer
  pJ360
agctgcaagaactcttcctcacg

SEQ ID NO: 23 - DNA, 24 bp, Artificial Sequence
  Primer
  pJ355
gtctcctttcagactgattccatg

SEQ ID NO: 24 - DNA, 62 bp, Artificial Sequence
  Allele- Fig 2B
  Endogenous slc35f2
tatccctgactgtatgaaccagtggcccacatttgtaatcctagcacctgggaggtagaaat

SEQ ID NO: 25 - DNA, 62 bp, Artificial Sequence
  Allele- Fig 2B
  Endogenous slc35f2
atagggactgacatacttggtcaccgggtgtaaacattaggatcgtggaccctccatcttta

SEQ ID NO: 26 - DNA, 71 bp, Artificial Sequence
  Allele- Fig 2B
  Endogenous slc35f2
tcccggagacccactctgcagtcttagtggacaagccacagactctggcatgtcaccaggagagagcagaa

SEQ ID NO: 27 - DNA, 71 bp, Artificial Sequence
  Allele- Fig 2B
  Endogenous slc35f2
agggcctctgggtgagacgtcagaatcacctgttcggtgtctgagaccgtacagtggtcctctctcgtctt

SEQ ID NO: 28 - DNA, 117 bp, Artificial Sequence
  Allele- Fig 2B
  RT slc35f2
gtgcttcgatatcgatgctttggagactgtatgaaccagtggcccacatttgtaatcctagcagaagttcctattccgaagttcctattcttcaaatagtataggaacttccctggg

SEQ ID NO: 29 - DNA, 117 bp, Artificial Sequence
  Allele- Fig 2B
  RT slc35f2
cacgaagctatagctagcaaacctctgacatacttggtcaccgggtgtaaacattaggatcgtcttcaaggataaggcttcaaggataagaagtttatcatatccttgaagggaccc

SEQ ID NO: 30 - DNA, 131 bp, Artificial Sequence
  Allele- Fig 2B
  RT slc35f2
cccttccctgtccttgaagttcctattccgaagttcctattctctagaaagtataggaacttcgctagagctggacaagccacagactctggcatgtcaccaggagagccaaacgatcgatatcgaagcac

SEQ ID NO: 31 - DNA, 131 bp, Artificial Sequence
  Allele- Fig 2B
  RT slc35f2
gggaagggacaggaacttcaaggataaggcttcaaggataagagatctttcatatccttgaagcgatctcgacctgttcggtgtctgagaccgtacagtggtcctctcggtttgctagctatagcttcgtg

SEQ ID NO: 32 - DNA, 117 bp, Artificial Sequence
  Allele- Fig 5
  Edited sk35f2
agcatgtatgaagctttatatccctagactgtatgaaccagtggcccacatttgtaatcctagcagaagttcctattccgaagttcctattcttcaaatagtataggaacttccctg

SEQ ID NO: 33 - DNA, 133 bp, Artificial Sequence
  Allele- Fig 5
  Edited sk35f2
ccttccctgtccttgaagttcctattccgaagttcctattctctagaaagtataggaacttcgctagagctggacaagccacagactctggcatgtcaccaggagagccaaacgatcgatatcgaagcaccta

SEQ ID NO: 34 - DNA, 32 bp, Artificial Sequence
  Allele- Fig 5
  Deleted
ccactctgcagtcttctagctggacaagccac

SEQ ID NO: 35 - DNA, 50 bp, Artificial Sequence
  Allele- Fig 5
  Edited sk35f2
ggagacccactctgcagactctggcatgtcaccaggagagagcagaacct

SEQ ID NO: 36 - DNA, 2922 bp, Artificial Sequence
  Locus- Fig 6A
  Edited slc35f2
gggtctctctcatgatgtgcatagttgccaattagttattccattttgtgagacagcttttctgatggaatccagtacgtggttcactatagattcttcccaaaggcctaggggatgcagctcatttggtagtctattggtttagcatgtatgaagcttatatccctagactgtatgaaccagtggcccacatttgtaatcctagcagaagttcctattccgaagttcctattcttcaaatagtataggaacttccctgggaggtagaaataggaggatcagacatccatggtcagccttgactacccagtgagtctgagatctgcctgggctacctgcgaccctgtcacaaacaaacaaaagccaaggatccagttagctstcaagccatctgagtccagtatttacctgtgtataggggagggctggggtattgtcatctcctatgatgacttccagaaagagactgtgaaggtagatgtcaaagaacagctgacaacaggccacagccccttgcttgtatgagggagtgcaactaactgcctgttttctctccacagttctcaggactctacatcctgtcgttcacagtcatcatggttggcttcatcttstactgttccaccccaacgcgcacggtagagcccccagaaagcagcgtgccccctgttaccagcatcgggatcgacaatttgggactgaagcttgaggagagtggtctcccggagacccactctgcagtcttggaattcgagaacctgtacttcccagagtggaggttccggcgggcatcatcaccaccatcacgaacaaaaactcatctcagaagaggatctgggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttcccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctccttgataaatatgcagcggacgaagatgccttctttgctgattacgctgaggctcaccaaaagctttccgagcttgggtttgctgatgccacgcgtgactacaaagaccatgacggtgattataaagatcatgacatcgattacaaggatgacgatgacaagggaggttccggcgggtacccttacgacstacctgattatgcctatccctatgatgtacccsactatgcataaggaacccggaaggccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcaacaaccccagtgccacgttgtgagttggatagttgtggaaagagtcaaatggctctcctcaagcctattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacacacgataataccatgctgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccata

SEQ ID NO: 37 - DNA, 1709 bp, Artificial Sequence
  Locus- Fig 6B
  Edited slc35f2- Before Linker
  misc_feature (639)..(639): n is a, c, g, or t
  misc_feature (1223)..(1223): n is a, c, g, or t
  misc_feature (1234)..(1234): n is a, c, g, or t
  misc_feature (1248)..(1248): n is a, c, g, or t
ttggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaaccggatagtgaaacaggggcaatggtgtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtgntgtgcactgtgtttgctgacgcaaccccactggttggggcattgccaccacctgtcagctcctttccgggacttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggnaagacaacctntagggcctgcgggntctattgggaaccaagctggagtgcagtggcacaatcttggctcactgcaatctccgcctcctgggttcaagcgattctcctgcctcagcctcccgagttgttgggattccaggcatgcatgatcaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaagttcctattccgaagttcctattctctagaaagtataggaacttcgctagagctggacaagccacagactctggcatgtcaccaggagagccaaacgatcgatatcgaagcacctagcggatctgacggttcactaaaccagctctgcttatatagacctcccaccgtacacgcctaccg

SEQ ID NO: 38 - DNA, 49 bp, Artificial Sequence
  Locus- Fig 6B
  Edited slc35f2- After Linker
gagacccactctgcagactctggcatgtcaccaggagagagcagaacct

SEQ ID NO: 39 - DNA, 63 bp, Artificial Sequence
  Locus- Fig7B
  Endogenous slc35f6
ttgctagccgtgggtttggaaaccaatgagttgtcatttgagtgacatcgaggcagagggaca

SEQ ID NO: 40 - DNA, 63 bp, Artificial Sequence
  Locus- Fig7B
  Endogenous slc35f6
aacgatcggcacccaaagctttggttactcaacagtaaactcactgtagctccgtctccctgt

SEQ ID NO: 41 - DNA, 74 bp, Artificial Sequence
  Locus- Fig7B
  Endogenous slc35f6
ggagagcaagagagactgctaggtgacagtcggactcccatcaatgaagccagctgagcccgcagtgagatctc

SEQ ID NO: 42 - DNA, 74 bp, Artificial Sequence
  Locus- Fig7B
  Endogenous slc35f6
cctctcgttctctctgacgatccactgtcagcctgagggtagttacttcggtcgactcgggcgtcactctagag

SEQ ID NO: 43 - DNA, 119 bp, Artificial Sequence
  Locus- Fig7B
  RT slc35f6
gtgcttccgatatcgatcgtttggccgtgggtttggaaaccaatgagttgtcatttgagtgacagaagttcctattccgaagttcctattcttcaaatagtataggaacttctcgaggc

SEQ ID NO: 44 - DNA, 119 bp, Artificial Sequence
  Locus- Fig7B
  RT slc35f6
cacgaaggctatagctagcaaaccggcacccaaacctttggttactcaacagtaaactcactgtcttcaaggataaggcttcaaggataagaagtttatcatatccttgaagagctccg

SEQ ID NO: 45 - DNA, 139 bp, Artificial Sequence
  Locus- Fig7B
  RT slc35f6
ccactgctcccttccctgtccttgaagttcctattccgaagttcctattctctagaaagtataggaacttcgctagagtcggactcccatcaatgaagccagctgagcccgcagtgccaaacgatcgatatcgaagcac

SEQ ID NO: 46 - DNA, 139 bp, Artificial Sequence
  Locus- Fig7B
  RT slc35f6
ggtgacgagggaagggacaggaacttcaaggataaggcttcaaggataagagatctttcatatccttgaagcgatctcagcctgagggtagttacttcggtcgactcgggcgtcacggtttgctagctatagcttcgtg

SEQ ID NO: 47 - DNA, 113 bp, Artificial Sequence
  Allele- Fig10
  5 prime cut
ggcttgagttgctagccgtgggtttggaaaccaatgagttgtcatttgagtgacagaagttcctattccgaagttcctattcttcaaatagtataggaacttctcgaggcaga

SEQ ID NO: 48 - DNA, 164 bp, Artificial Sequence
  Allele- Fig10
  3 prime cut
actgctcccttccctgtccttgaagttcctattccgaagttcctattctctagaaagtataggaacttcgctagccttccaagtattatcagtcttgttacatgataattaagactcccatcaatgaagccagctgagcccgcagtgagatctctgctgccacc

SEQ ID NO: 49 - DNA, 3419 bp, Artificial Sequence
  Locus- Fig 11A
  Edited slc35f6
tatacacttcctctagagggagcgacagccaggaaggggccaaacagtgcacctgagcttggaccagttatactcagaacctcactgctgagcagttctgagtctctggagcacagctcccgacaggcagggggaaagaagggggcatcctgacagggcacagtggcttgagttgctagccgtgggtttggaaaccaatgagttgtcatttgagtgacagaagttcctattccgaagttcctattcttcaaatagtataggaacttctcgaggcagagggacagatcctgagagtggacaacagaaacctaacaaagacagtggtcagagatgagagtgcctgtgggccatgctgggagcccaggcagggtgagaatgtgatgcacgtgtctgcttcccccaggcttcttcggcttcgtgatcctctcgctgctccttgtgcctatgttctacatcccacggcctcctttagtgggaaccctcgcggggtgctggaagacgcgctggatgccttctgccaagtgggccgacagcccctgattgccctggctctgctaggcaacatcagcagcattgccttcttcaacttttcgggcatcagcgtcaccaaggaactgagtgccaccacccgcatggtgctagacaccctgcgcaccatagtcatctgggcctttaccctcgcactgggctgggagatcttctatccattgcagatccttggtttcctcatcctccttatgggcaccgccctctacaatgggctgcaccgaccactgctagccttcctgtccagacgctggcgactccctacccaggagggagagcaagagagactgctaggtgacagccgaacccctattaacgaggcgagtgaattcgagaacctgtacttccagagtggaggttccggcgggcatcatcaccaccatcacgaacaaaaactcatctcagaagaggatctgggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctatttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgataaatatgcagcggacgaagatgccttctttgctgattacgctgaggctcaccaaaagctttccgagcttgggtttgctgatgccacgcgtgactacaaagaccatgacggtgattataaagatcatgacatcgattacaaggatgacgatgacaagggaggttccgcgggtacccttacgacgtacctgattatgcctatccctatgatgtacccgactatgcataaggaacccggaaggccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacacacgataataccatgctgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccgtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatga

SEQ ID NO: 50 - DNA, 1538 bp, Artificial Sequence
  Locus- Fig 11B
  Edited slc35f6
actatatccgtaacctggatagtgaaacaggggcaatggtgtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctccttccttgtataaatcctggttgctgtctctttatgaggagttgtggcccgttgtcaggcaacgtggcgtggtgtgcactgtgtttgctgacgcaacccccactggttggggcattgccaccacctgtcagctccttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttgaagttcctattccgaagttcctattctctagaaagtataggaacttcgctagccttccaagtattatcagtcttgttacatgataattaagactcccatcaatgaagccagctgagcccgcagtgagatctctgctgccaccagatgctgaggccatgcagactggtgggctgtgggtgccctgtccccaaggcctcaccccacccatccctaatgacccctctgtgcggccgttgccagagaggatgggaggataccaggtcttgcttaatcctgaggcctgtaatattgggacccagcccccacaggcccagagatggcaaccactctcagcctccagttctgctgcactagaacaaaattacgagcttgaatggcaagaattcac

SEQ ID NO: 51 - DNA, 124 bp, Artificial Sequence
  Allele- Fig 12A
  MED13L
gcaaactgctacgtcacgggatcatgcccgtcccaaaaactgtcccacacaagctcacagatcagcttccagtcctttaaccagccccacacgcgcaagtttcccgccatcacctaggtaaatg

SEQ ID NO: 52 - protein, 64 aa, Artificial Sequence
  Allele- Fig 12A
  MED13L Exon 20 Amino Acid
MCRLGQHKPICKVLRDGIMRVGKTVAQKLTDELVSEQFNQPWSGEENDNHSRLKLYAQVCRHHL

SEQ ID NO: 53 - DNA, 20 bp, Artificial Sequence
  Allele- Fig 12A
  MED13L WT
tgacaatcattccagactca

SEQ ID NO: 54 - DNA, 21 bp, Artificial Sequence
  Allele- Fig 12A
  MED13L Mutant
tgacaatcatttccagactca

SEQ ID NO: 55 - protein, 6 aa, Artificial Sequence
  Allele- Fig 12A
  MED13L Mutant Amino Acid >
DNMFQT

SEQ ID NO: 56 - DNA, 126 bp, Artificial Sequence
  Allele- Fig 12C
  MED13L Mutant 5′
attacaattacctgaaaggaccaacttgtaatgttggtttgattgcatgtgataaccctaaaagaaaaaggaaaatgcttccatatgtatgttaaagaatctcccttgctaaccattggtactgaa

SEQ ID NO: 57 - DNA, 126 bp, Artificial Sequence
  Allele- Fig 12C
  MED13L Mutant 5′
taatgttaatggactttcctggttgaacattacaaccaaactaacgtacactattgggattttctttttccttttacgaaggtatacatacaatttcttagagggaacgattggtaaccatgactt

SEQ ID NO: 58 - DNA, 124 bp, Artificial Sequence
  Allele- Fig 12C
  MED13L Mutant 3′
gtcccatcccttcttttatagatgcaacattatatttcttttgtctcaagtaatgctgtatttgttaaggtccagtttggtgttctgggcagtttaatgctaatttctatatggacatatattt

SEQ ID NO: 59 - DNA, 124 bp, Artificial Sequence
  Allele- Fig 12C
  MED13L Mutant 3′
cagggtagggaagaaaatatctacgttgtaatataaagaaaacagagttcattacgacataaacaattccaggtcaaaccacaagacccgtcaaattacgattaaagatatacctgtatataaa

SEQ ID NO: 60 - DNA, 141 bp, Artificial Sequence
  Allele- Fig 12C
  MED13L RT 5′
cgattgtgcttcgatatcgatcgtttgggaaaggaccaacttgtaatgttggtttgattgcatgtgataaccctaaaagaaaaaggaaaatgcttccatatgtatgttaaagaatctcccttgctaaccatttttactgaa

SEQ ID NO: 61 - DNA, 141 bp, Artificial Sequence
  Allele- Fig 12C
  MED13L RT 5′
gctaacacgaagctatagctagcaaaccctttcctggttgaacattacaaccaaactaacgtacactattgggattttctttttccttttacgaaggtatacatacaatttcttagagggaacgattggtaaaaatgactt

SEQ ID NO: 62 - DNA, 144 bp, Artificial Sequence
  Allele- Fig 12C
  MED13L RT 3′
tcccataaattcttttatagatgcaacattatatttcttttgtctcaagtaatgctgtatttgttaaggtccagtttggtgttctgggcagtttaatgctaatttctatatgccaaacgatcgatatcgaagcacaatcactag

SEQ ID NO: 63 - DNA, 144 bp, Artificial Sequence
  Allele- Fig 12C
  MED13L RT 3′
agggtatttaagaaaatatctacgttgtaatataaagaaaacagagttcattacgacataaacaattccaggtcaaaccacaagacccgtcaaattacgattaaagatatacggtttgctagctatagcttcgtgttagtgatc

SEQ ID NO: 64 - DNA, 153 bp, Artificial Sequence
  Allele- Fig 14
  MED13L 5′
ggtacattacaattacctgaaaggaccaacttgtaatgttggtttgattgcatgtgataaccctaaaagaaaaaggaaaatgcttccatatgtatgttaaagaatctcccttgctaacctttgcgcagggacgcggctgctctgggcgtggtt

SEQ ID NO: 65 - DNA, 50 bp, Artificial Sequence
  Allele- Fig 14
  MED13L Puromycin 3′
  misc_feature (47)..(47): n is a, c, g, or t
tgcccgaaggaccgcgcacctggtgcatgacccgcaagcccggtgcntga

SEQ ID NO: 66 - DNA, 54 bp, Artificial Sequence
  Allele- Fig 14
  MED13L Exon20 3′
atgacaatcattccagactcaaactttatgcgcaagtttgccgccatcacctag

SEQ ID NO: 67 - DNA, 176 bp, Artificial Sequence
  Allele- Fig 14
  MED13L 3′
gtaaatgggaatttatttggcaaagtagaattggagaagtaaaatagcaaatccgtcccataaattcttttatagatgcaacattatatttcttttgtctcaagtaatgctgtatttgttaaggtccagtttggtgttctgggcagtttaatgctaatttctatatggacatatat

SEQ ID NO: 68 - DNA, 634 bp, Artificial Sequence
  Allele- Fig 15A
  MED13L Mutant Restored
gtagattacaattacctgaaaggaccaacttgtaatgttggtttgattgcatgtgataaccctaaaagaaaaaggaaaatgcttccatatgtatgttaaagaatctcccttgctaacctttgcgcagggacgcggctgctctgggcgtggttccgggaaacgcagcggcgccgacccgggactcgcacattcttcacgtccgttcgcagcgtcacccggatcttcgccgctacccttgtgggccccccggcgacgcttcctgctccgcccctaagtcgggaaggttccttgcgttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggt

SEQ ID NO: 69 - DNA, 736 bp, Artificial Sequence
  Allele- Fig 15B
  MED13L Mutant Restored
ccgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccactttctagagtcggggcggccggccgcttcgagcagacatgataagatacattga

SEQ ID NO: 70 - DNA, 541 bp, Artificial Sequence
  Allele- Fig 15C
  MED13L Mutant Restored
tgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcaggctaatgatcttgtctatgatatcagatgtgtaggcttgggcagcacaagcccatctgcaaagtgctacgtgacgggatcatgcgcgtgggaaaaactgtggcacagaagctgacagatgagcttgtgagtgagtggtttaaccagccttggagcggcgaggagaatgacaatcattccagactcaaactttatgcgcaagttttgccgccatcacctaggtaaatgggaatttatttggcaaagtagaattggagaagtaaaatagcaaatccgtcccataaattctttatagatgcaacattatatttcttttgtctcaagtaatgctgtatttgttaaggtccagtttggtgttctgggcagtttaatgctaatttctatatgacatata

SEQ ID NO: 71 - DNA, 125 bp, Artificial Sequence
  Allele- Fig 15D
  MED13L Deleted Sequence
atttttactgaatgaaggaatggctcctgttataactaatgatcttgtctatgatgacagcagagatccactttggccgcggctcgagggggttggggttgcgccttttccaaggcagccctggg

SEQ ID NO: 72 - DNA, 126 bp, Artificial Sequence; identical to SEQ ID NO: 56
  Allele- Fig 16B
  MED13L Endogenous 5′

SEQ ID NO: 73 - DNA, 126 bp, Artificial Sequence; identical to SEQ ID NO: 57
  Allele- Fig 16B
  MED13L Endogenous 5′

SEQ ID NO: 74 - DNA, 124 bp, Artificial Sequence; identical to SEQ ID NO: 58
  Allele- Fig 16B
  MED13L Endogenous 3′

SEQ ID NO: 75 - DNA, 124 bp, Artificial Sequence; identical to SEQ ID NO: 59
  Allele- Fig 16B
  MED13L Endogenous 3′

SEQ ID NO: 76 - DNA, 126 bp, Artificial Sequence; identical to SEQ ID NO: 56
  Allele- Fig 16B
  MED13L RT 5′

SEQ ID NO: 77 - DNA, 126 bp, Artificial Sequence; identical to SEQ ID NO: 57
  Allele- Fig 16B
  MED13L RT 5′

SEQ ID NO: 78 - DNA, 124 bp, Artificial Sequence; identical to SEQ ID NO: 58
  Allele- Fig 16B
  MED13L RT 3′

SEQ ID NO: 79 - DNA, 124 bp, Artificial Sequence; identical to SEQ ID NO: 59
  Allele- Fig 16B
  MED13L RT3′

SEQ ID NO: 80 - DNA, 259 bp, Artificial Sequence
  Allele- Fig 18
  MED13L 5′
ttttttaaagagcctagtccaagttttaaagagacagatagcaatttaagggtacattacaattacctgaaaggaccaacttgtaatgtttgcatgtgataaccctaaaaaaaaaaggaaaatgcttccttatgtatgttaaaaaatctcccttgctaaccattggtactgaatgaaggaatggctcctgttataactaatgatcttgtctatgatgacagcaaaaatccactttggccgcggctcgagggggttgggg

SEQ ID NO: 81 - DNA, 178 bp, Artificial Sequence
  Allele-Fig 18
  MED13L 3′
gtaaatgggaatttatttggcaaagtagaattggagaagtaaaatagcaaatccgtcccataaattcttttatagatgcaacattatatttcttttgtctcaagtaatgctgtatttgttaaggtccagtttggtgttctgggcagtttaatgctaatttctatatggacatatattt

SEQ ID NO: 82 - DNA, 729 bp, Artificial Sequence
  Allele- FIG19A
  MED13L Mutant Restored
ttttaaagagcctagtccaagttttaaagagacagatagcaatttaagggtacattacaattacctgaaaggaccaacttgtaatgtttgcatgtgataaccctaaaaaaaaaaggaaaatgcttccttatgtatgttaaaaaatctcccttgctaacattggtactgaatgaaggaatggctcctgttataactaatgatcttgtctatgatgacagcaaaaatccactttggccgcggctcgagggggttggggttgcgccttttccaaggcagccctgggtttgcgcagggacgcggctgctctgggcgtggttccgggaaacgcagcggcgccgaccctgggactcgcacattcttcacgtccgttcgcagcgtcacccggatcttcgccgctacccttgtgggccccccggcgacgcttcctgctccgcccctaagtcgggaaggttcctgcggttcgcggcgtgccggacgtgacaaacggaagccgcacgtctcactagtaccctcgcagacggacagcgccagggagcaatggcagcgcgccgacccgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagccccggagcgcacgtcggcagcggctccctcgttgac

SEQ ID NO: 83 - DNA, 736 bp, Artificial Sequence
  Allele- FIG19B
  MED13L Mutant Restored
cgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctg

SEQ ID NO: 84 - DNA, 595 bp, Artificial Sequence
  Allele- FIG19C
  MED13L Mutant Restored
tagatcttagccactttctagagtcgggccggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgttcaggttcaggctaatgacttgtctatgatatcagatgtgtaggctgggcagcacaagcccatctgcaaagtgctacgtgacgggatcatgcgcgtgggaaaaactgtggcacagaagctgacagatgagcttgtgagtgagtggtttaaccagccttggagcggcgaggagaatgacaatcattccagactcaaactttatgcgcaagtttgcccatcacctaggtaaatgggaatttatttggcaaagtagaattggagaagtaaaatagcaaatccgtcccatcccttcttagatgcaacattttgtctcaagtaatgctgtatttgttaaggtccagtttggtgttctgggcagtttaatgctaatttctatatggacatatattt

SEQ ID NO: 85 - DNA, 510 bp, Artificial Sequence
  Allele- FIG20A
  MED13L
aatttaagggtacattacaattacctgaaaggaccaacttgtaatgttggtttgattgcatgtgataaccctaaaagaaaaaggaaaatgcttccatatgtatgttaaagaatctcccttgctaaccattggtactgaatgaaggaatggctcctgttataactaatgatcttgtctatgatatcagatgtgtaggcttgggcagcacaagcccatctgcaaagtgctacgtgacgggatcatgcgcgtgggaaaaactgtggcacagaagctgacagatgagcttgtgagtgagtggtttaaccagccttggagcggcgaggagaatgacaatcattccagactcaaactttatgcgcaagtttgccgccatcacctaggtaaatgggaatttatttgggaaagtagaattggagaagtaaaatagcaaatccgtcccatcccttcttttatagatgcaacattatatttcttttgtctcaagtaatgctgtatttgttaaggtccagt

SEQ ID NO: 86 - DNA, 510 bp, Artificial Sequence
  Allele- FIG20A
  MED13L Complement
ttaaattcccatgtaatgttaatggactttcctggttgaacattacaaccaaactaacgtacactattgggattttctttttccttttacgaaggtatacatacaatttcttagagggaacgattggtaaccatgacttacttccttaccgaggacaatattgattactagaacagatactatagtctacacatccgaacccgtcgtgttcgggtagacgtttcacgatgcactgccctagtacgcgcaccctttttgacaccgtgtcttcgactgtctactcgaacactcactcaccaaattggtcggaacctcgccgctcctcttactgttagtaaggtctgagtttgaaatacgcgttcaaacggcggtagtggatccatttacccttaaataaaccgtttcatcttaacctcttcattttatcgtttaggcagggtagggaagaaaatatctacgttgtaatataaagaaaacagagttcattacgacataaacaattccaggtca

SEQ ID NO: 87 - DNA, 31 bp, Artificial Sequence
  Locus- FIG20C
  Cas9 target 5′
ttaaagaatctcccttgctaaccattggtac

SEQ ID NO: 88 - DNA, 31 bp, Artificial Sequence
  Locus- FIG20C
  Cas9 target 5′
aatttcttagagggaacgattggtaaccatg

SEQ ID NO: 89 - DNA, 85 bp, Artificial Sequence
  Locus- FIG20C
  iCAP Cas9 deletion
aaatgcttccatatgtatgttaaagaatctcccttgctaacttttatagatgcaacattatatttcttttgtctcaagtaatgct

SEQ ID NO: 90 - DNA, 39 bp, Artificial Sequence
  Locus- FIG20C
  Cpf1 target 5′
ttgtaatgttggtttgattgcatgtgataaccctaaaag

SEQ ID NO: 91 - DNA, 39 bp, Artificial Sequence
  Locus- FIG20C
  Cpf1 target 5′
aacattacaaccaaactaacgtacactattgggattttc

SEQ ID NO: 92 - DNA, 91 bp, Artificial Sequence
  Locus- FIG20C
  iCAP Cpf1 deletion
aagggtacattacaattacctgaaraggaccaacttgtaatgttggttttgtctcaagtaatgctgtatttgttaaggtccagtttggtgt

SEQ ID NO: 93 - DNA, 31 bp, Artificial Sequence
  Locus- FIG20C
  Cas9 target 3′
catcccttcttttatagatgcaacattatat

SEQ ID NO: 94 - DNA, 31 bp, Artificial Sequence
  Locus- FIG20C
  Cas9 target 3′
gtagggaagaaaatatctacgttgtaatata

SEQ ID NO: 95 - DNA, 42 bp, Artificial Sequence
  Locus- FIG20C
  Cpf1 target 3′
tttatagatgcaacattatatttcttttgtctcaagtaatgc

SEQ ID NO: 96 - DNA, 42 bp, Artificial Sequence
  Locus- FIG20C
  Cpf1 target 3′
aaatatctacgttgtaatataaagaaaacagagttcattacg

(19) United States
(12) Patent Application Publication
(10) Pub. No.: US 2024/0100184 A1
(43) Pub. Date: Mar. 28, 2024

(54) METHODS OF PRECISE GENOME EDITING BY IN SITU CUT AND PASTE (ICAP)

(71) Applicant: Rowan University, Glassboro, NJ (US)

(72) Inventors: Ping Jiang, Bryn Mawr, PA (US); Kevin M. Kemper, Voorhees, NJ (US); Salvatore J. Caradonna, Voorhees, NJ (US)

(21) Appl. No.: 17/767,944

(22) PCT Filed: Oct. 20, 2020
(86) PCT No.: PCT/US2020/056453

§ 371 Date: Apr. 11, 2022

Related U.S. Application Data
(60) Provisional application No. 62/923,727, filed on Oct. 21, 2019.

(51) Int. Cl. A61K 48/00; A61K 31/7088; A61K 38/46; C12N 9/22; C12N 15/11; C12N 15/90

(52) U.S. Cl. CPC A61K 48/0016; A61K 31/7088; A61K 38/465; C12N 9/22; C12N 15/11; C12N 15/907; C12N 2310/20; C12N 2800/80

(57) ABSTRACT

The present invention relates to innovative means of DNA sequence editing involving in-situ cut-and-paste (iCAP) or alternatively cut-and-paste in-situ (CAPi). Thus, in various embodiments described herein, the methods of the invention relate to methods of generating paired-end nucleic acid fragment sharing common linker nucleic acid sequences using a nicking endonuclease, a T7 endonuclease, a restriction enzyme or a transposase, methods of analyzing the nucleotides sequences from the linked-paired-end sequenced fragments and methods of de novo whole genome mapping.

### CROSS-REFERENCE TO RELATED APPLICATION

The present application claims priority under 35 U.S.C. § 119(e) to U.S. Provisional Patent Application 62/923,727, filed Oct. 21, 2019, which is hereby incorporated by reference in its entirety.

### BACKGROUND OF THE INVENTION

Genome editing has broad applications in biomedical research and clinical therapeutics. Genome editing has been significantly facilitated by the Crispr/Cas system, but an intentional and targeted editing technique capable of inducing several types of precise nucleotide alterations (i.e., single base additions, deletions, and/or exchanges) at precise locations in mammalian genomes remains to be a challenging task.

Thus, despite all developments in genome manipulation, there remains a need in the art for a method of precise genome editing that simply utilizes a single platform for achieving multiple different types of alterations (i.e., nucleotide alterations at a single base level, precise deletion or addition of DNA sequence fragments, and so forth) and alterations of multiple sites simultaneously, as most of existing methods are able to make single edits or require complicated, programmable nuclease derivatives. The present invention addresses these needs.

### SUMMARY OF THE INVENTION

The present invention relates to innovative means of DNA sequence editing involving in-situ cut-and-paste (iCAP) or alternatively cut-and-paste in-situ (CAPi). Thus, in various embodiments described herein, the methods of the invention relate to methods of generating paired-end nucleic acid fragment sharing common linker nucleic acid sequences using a nicking endonuclease, a T7 endonuclease, a restriction enzyme or a transposase, methods of analyzing the nucleotides sequences from the linked-paired-end sequenced fragments and methods of de novo whole genome mapping.

Thus, in some aspects, the invention includes a method of editing, mutating, or modifying a genomic target DNA sequence in a cell. In certain embodiments, the method comprises providing (i) a DNA replacement template (dRT) comprising the target DNA sequence comprising the desired edited, mutated, or modified nucleotide(s), and (ii) a sequence encoding a nuclease. In certain embodiments, the method comprises contacting the genomic target DNA sequence, the DNA RT, and heterologous guide-RNAs (gRNAs) under conditions that allow for the gRNAs to induce double-strand breaks of the genomic target DNA sequence and the RT by the nuclease generating either blunt ends or overhanging ends. In certain embodiments, the method comprises subjecting the blunt ends of the genomic target DNA sequence and DNA RT to 5′ to 3′ DNA end resection to generate complementary 3′ overhangs. In certain embodiments, the method comprises annealing 3′ complementary overhangs of the DNA replacement template to the complementary 3′ overhang sequences of the target DNA sequence. In certain embodiments, the method comprises ligating the annealing sites, thereby resulting in incorporation of the DNA RT into the genomic target DNA sequence.

In certain embodiments, the method further comprises subjecting the overhanging ends flanking the endogenous genomic target DNA sequence and the DNA RT to modification resulting in blunt ends. In certain embodiments, the method comprises ligating the blunt ends of RT to the genomic target DNA sequence, thereby resulting in incorporation of the DNA RT into the place of the genomic target DNA sequence.

In certain embodiments, the blunt ends generated by the nuclease or resulting from modification of the overhanging ends of the target DNA sequence are ligated together, thereby resulting in the deletion of the target DNA sequence.

In certain embodiments, the nuclease is a Cas9 nuclease.

In certain embodiments, the Cas9 nuclease is a naturally-occurring variant thereof.

In certain embodiments, the Cas9 nuclease variant comprises SpCas9, SaCas9, StCas9, NmCas9, FnCas9, CjCas9, CasX, CasY, Cas12a, Cas14a, BlCas9, ScCas9, LmoCas9, TdCas9, Nme2Cas9, GsCas9, BlatCas9, FnCas9-RHA.

In certain embodiments, the Cas12a nuclease variant comprises AsCpf1, FnCpf1, LbCpf1, AsCpf1-RR, LbCpf1-RR, AsCpf1-RVR.

In certain embodiments, the nuclease is a Cas variant nuclease.

In certain embodiments, the Cas variant nuclease comprises Cas13a/b(C2c2), Cas12b(C2c1), Cas12c(C2c3).

In certain embodiments, the Cas9 nuclease is an engineered variant thereof.

In certain embodiments, the Cas9 nuclease variant comprises eSpCas9, SpCas9-HF1, Fok1-Fused dCas9, xCas9, SpCas9-VQR, SpCas9-VRER, SpCas9-D1135E, SpCas9-EQR, SpCas9-QQR1, Cas9-DD, HypaCas9, evoCas9, xCas9-3.7, SniperCas9, Cas9-CtIP, SpCas9-NG, Split-SpCas9, SpCas9-K855A, ScCas9+, ScCas9++, SaCas9-KKH, SaCas9.

In certain embodiments, the cell is a eukaryotic cell.

In certain embodiments, the eukaryotic cell is a mammalian cell.

In certain embodiments, the mammalian cell is part of a tissue or organism and the method is performed in situ.

In certain embodiments, the mammalian cell is a developing embryo.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1D are diagrams depicting a non-limiting schematic of the iCAP process. FIG. 1A depicts an overview of the iCAP genome editing method. FIG. 1B is a diagram depicting a non-limiting schematic of the iCAP process involving DNA replacement template (dRT) containing mini-homology sequences (overhang substrate sequences) in the edited isogenic fragment. This strategy is called Design A. FIG. 1C is a diagram depicting a non-limiting schematic of the iCAP process involving DNA replacement template (dRT) without mini-homology sequences (overhang substrate sequences) in the edited isogenic fragment. This strategy is called Design B). FIG. 1D is a diagram depicting a non-limiting schematic of the iCAP process involving precise deletion of a section of endogenous genome sequences without being replaced.

FIGS. 2A-2F are diagrams illustrating the design of a proof of concept experiment involving editing the Slc35f2 locus in the mouse genome using iCAP. FIG. 2A is a diagram overview of the study. The mouse Slc35f2 locus (top), the DNA replacement template APEX2-IRES-CRE (middle), and the edited locus resulting from the iCAP process (bottom). FIG. 2B is an illustration of the endogenous Slc35f2 genomic DNA sequences in the areas flanking the gRNA target sequence and the same (isogenic) sequence areas in the pre-constructed DNA replacement template. The two genomic gRNA targets for Slc35f2 are shown (top), and Cas9 DSB sites for each of the gRNA targets are marked by an black vertical arrows (referred to as inner cuts). Engineered gRNA targets in dRT are shown in bottom, and Cas9 DSB sites for each of the gRNA targets are marked by an blue vertical arrows (referred to as outer cuts). In the DNA replacement template, the upstream genomic gRNA target site in the intron upstream of the last exon is disrupted by insertion of a FRT3 site while the downstream genomic gRNA target site in the area of the stop codon (octagon) is disrupted by in-frame insertion of an exogenous expression cassette. Shaded areas of the gene's sequence, both upstream & downstream, are the 5′ & 3′ 40 bp overhang substrate sequences (homology regions), which are also built into the DNA replacement template. The engineered gRNA target sites (arrowed boxes) built into the DNA replacement template and flanking 5′ & 3′ overhang substrates are shown. Nucleotide sequence in black frame is an NheI restriction site. FIG. 2C is an illustration of the 3′ genomic region for slc35f2 showing the position of the final two exons (white boxes), position of the endogenous STOP codon (octagon), validated “inner” gRNA target sites #1 & #2 (black arrows), and position of primers (For/Rev) for creation of a Cas9 assay PCR fragment & genotyping (“forward” and “reverse” arrows). gRNA #1 creates a DSB within the final intron of slc35f2 305 bp upstream of the final exon, and gRNA #2 creates a DSB at the endogenous STOP codon. FIG. 2D is an agarose gel showing results of an in vitro Cas9 assay. The 1024 bp PCR fragment generated using the primers shown above was cleaved by the active sgRNA/Cas9 enzyme complex in Lane 1, resulting in 3 bands (492 bp, 324 bp & 208 bp; black arrows). Lane 2 shows the uncleaved PCR fragment. Lane 3 is a DNA size ladder. FIG. 2E is a map of the 9466 bp iCAP plasmid containing DNA replacement template (donor construct) for slc35f2. Position of Cas9 target sites (arrows), flanking FRT sites (dark arrowed boxes), final exon (white arrowed box), in-frame expression cassette (APEX2 tag, 3×FLAG, 2HA, IRES, Cre-NLS, WPRE, hGH poly(A) signal), and relocated endogenous STOP codon (stippled octagon) are shown. DSB creation resulted in a 4386 bp edited isogenic fragment as the “patching” repair template. The position of CRISPR-Cas9 mediated DSBs are indicated by blue arrows. 40 bp overhang substrate regions (shaded boxes at either end of the insert region) between the Cas9 target site & FRT sites are regions that undergo 5′-to-3′ end resection following DSB generation, and are homologous to the similarly resected regions within the endogenous allele. FIG. 2F is an agarose gel showing the results of an in vitro Cas9 assay. The plasmid is cleaved by the active sgRNA/Cas9 enzyme complex in Lanes #1 & #3 resulting in two smaller fragments (5080 bp+4386 bp; black arrows). Lane 2 contained the same plasmid DNA with sgRNAs specific for only the genomic target sequences (therefore, no cleavage). Lane 4 contained uncut plasmid. Lane 5 contained a DNA size marker.

FIGS. 3A-3B are a table and image depicting the iCAP editing of the slc35f2 locus. FIG. 3A is a table listing a total of 74 one-cell stage embryos of strain B6D2F1 that were injected with a buffer mixture (Cas9 mRNA, 3 sgRNAs, and the DNA replacement template excised from a plasmid with restriction enzymes NdeI and XmaI). Surviving embryos were re-implanted into the oviduct of pseudo pregnant surrogate mothers for development to term. A total of 9 pups were born and genotypically analyzed with results that shows one animal (11%) carrying iCAP edited Slc35f2 allele and one animal (11%) carrying iCAP precise deletion. FIG. 3B is an agarose gel depicting a SURVEYOR nuclease assay testing for evidence of CRISPR/Cas9 mediated DSBs in genomes of the 9 animals.

FIGS. 4A-4B are a diagram and images showing the results of iCAP genome editing. FIG. 4A is a diagram showing the predicted structure of the successfully edited slc35f2 allele. Primers for genotyping the 5′ and 3′ ends of the inserted gene are indicated (“forward” and “reverse” arrows). The expected size of positive PCR products are 1768 bp (For/Rev 1) and 1850 bp (For/Rev 2). Vertical dashed lines indicate the limits of homology present in the DNA replacement template. FIG. 4B shows results of genotyping PCRs done on the 9 pups (A-I) for identification of successful cut and paste at the 5′ and 3′ DSBs, and animal G was identified.

FIG. 5 is a diagram of PCR & sequencing of Animal G revealing iCAP precise insertions (knock in) of the 5′ FRT3 site in the intron and the expression cassette with 3′ FRT site ending in frame with the last exon.

FIGS. 6A-6B are diagrams showing the sequence particularly at the 5′ and 3′ paste sites of the slc35f2 locus following iCAP editing and illustrated in FIG. 5. Shading indicates sequences corresponding to various features illustrated in previous figures.

FIGS. 7A-7F depict another iCAP study in which a 5′ FRT3 site and the APEX2-IRES-CRE expression cassette were inserted at two locations into the slc35f6 locus. FIG. 7A is a diagram of the endogenous slc35f6 genomic DNA sequence spanning exon5 to exon6 showing locations of genomic gRNA target sites (black triangles on “3′ region of exon 5-6”) for inner cuts (black arrows) and dRT (Design A) with engineerd gRNA target sites (triangles at either end of the template) for outer cuts (arrows) by Cas9. FIG. 7B is a more detailed diagram of the genomic and dRT sequences flanking the gRNA target sequences. Nucleotide sequence in black frame are the NheI restriction site. FIG. 7C is a diagram of the 3′ genomic region for slc35f6 showing the position of the final two exons (white boxes), position of the endogenous STOP codon (red octagon), validated “inner” gRNA target sites #1 & #2 (black arrows), and position of primers (For/Rev) for creation of a Cas9 assay PCR fragment & genotyping (“forward” and “reverse” arrows). FIG. 7D is an agarose gel showing results of an in vitro Cas9 assay. The 1105 bp PCR fragment generated using the primers shown above was cleaved by the active sgRNA/Cas9 enzyme complex in Lane 1, resulting in 3 bands (579 bp, 305 bp & 221 bp; black arrows). Lane 2 is a DNA size ladder. Lane 3 shows the uncleaved PCR fragment. FIG. 7E is a diagram of the 9581 bp plasmid containing DNA replacement template (DNA donor) for slc35f6 showing the position of the outer target sites (blue arrows), flanking 40 bp overhang substrate regions (orange boxes), flanking FRT sites (dark arrowed boxes), final exon (white arrowed box), in-frame expression cassette (APEX2 tag, 3×FLAG, 2HA, IRES, Cre NLS, WPRE, hGH poly(A) signal, and relocated endogenous STOP codon (octagon). FIG. 7F is an image of an agarose gel showing the results of an in vitro Cas9 assay. The plasmid is cleaved by the active and specific sgRNA/Cas9 enzyme complex in Lanes #1 & #3 resulting in two smaller fragments (5080 bp+4501 bp; black arrows). (Lane 1—plasmid DNA+(outer sgRNA+Cas9); Lane 2—plasmid DNA+(sgRNA #1+sgRNA #2+Cas9); Lane 3—plasmid DNA+(outer sgRNA+gRNA #1+gRNA #2+Cas9); Lane 4—Uncut plasmid; Lane 5—DNA size marker).

FIGS. 8A-8B are a table and image detailing the results of the iCAP genome editing. FIG. 8A is a table showing that a total of 65 one-cell stage embryos of strain B6D2F1 were injected with a buffer mixture (Cas9 mRNA, 3 sgRNAs, and the DNA replacement template excised from a plasmid with restriction enzymes NdeI and XmaI). Surviving embryos were re-implanted into the oviduct of pseudo pregnant surrogate mothers for development to term. A total of 16 pups were born. Genotyping by PCR & DNA sequencing was performed on biopsy samples collected at 3 weeks of age. FIG. 8B is a SURVEYOR Nuclease assay testing for evidence of CRISPR/Cas9 mediated DSBs. The assay revealed 13/16 animals (Animals A,B,D,E,F,G,H,J,L,M,N,O&P; 81%) showed signs of Cas9 mediated genome editing (arrows). Although Animal D did not produce any band in the SURVEYOR assay, PCR screening for the knock-in of the edited isogenic fragment released from RT were positive.

FIGS. 9A-9B are a diagram and image showing the results of the previous iCAP editing experiment. FIG. 9A is a diagram showing the predicted structure of the successfully edited slc35f6 allele. Primers for genotyping the 5′ and 3′ ends of the inserted gene are indicated (“forward” and “reverse” arrows). The expected size of positive PCR products are 1944 bp (For/Rev 1) and 1831 bp (For/Rev 2). Vertical dashed lines indicate the limits of homology present in the DNA replacement template. FIG. 9B is an agarose gel showing the results of genotyping PCRs done on the 16 pups (A-P) for identification of successful iCAP insertion of the replacement template. 1/16 pups (Animal D; 6%) showed positive for both PCR reactions that was proven by sequencing to have the edited isogenic fragment introduced into the slc35f6 gene via iCAP knock in.

FIG. 10 illustrates sequencing analysis of the iCAP-edited slc35f6 locus in Animal D that PCR screened positive for successful editing.

FIGS. 11A-11B illustrate the full sequence of the edited region at slc35f6 locus in Animal D. Sections of sequence corresponding to the various features are shaded as indicated in FIG. 11B.

FIGS. 12A-12G are diagrams illustrating the design of a proof of concept experiment involving editing the MED13L locus in the human genome using iCAP. FIG. 12A is a diagram of the human MED13L genomic locus showing the gene organization (top), the sequence of exon20 and amino acids coded (middle), and a single nucleotide base thymine duplication or insertion in exon20 on the mutant allele in patient cells (bottom). The single nucleotide addition causes S1497F mutation leading to reading-frame-shift and premature stop of transcription in exon 21, and results in clinic manifestations of MED13L syndrome. FIG. 12B is an overview diagram of the study using iCAP (Design A) to eliminate the single nucleotide duplication from exon20 of MED13L mutant allele. The exon20 region of human MED13L mutant allele (top), the DNA replacement template containing hPGK-Puromycin and wildtype exon 20 (middle), and the edited locus resulting from the iCAP process (bottom) are shown. FIG. 12C is an illustration of the endogenous MED13L genomic DNA sequences in the areas flanking the gRNA targeted sites on 5′ and 3′ sides of exon20 and the isogenic genomic sequence areas in the pre-constructed DNA replacement template. The two genomic gRNA targets are shown (top half, arrowed “Cas9 gRNA Target” bars), and Cas9 DSB sites for each of the gRNA targets (inner cuts) are marked by black vertical arrows. In bottom half of FIG. 12C, 100 bp sequences (shaded) as overhang substrates (mini-homology sequences matched to those of shaded endogenous regions in top half of the figure) are included in 5′ and 3′ ends of the edited isogenic fragment in DNA replacement template, respectively, and an engineered gRNA target (arrowed boxes) are placed on the 5′ end of the upstream overhang substrate and the 3′ end of the downstream overhang substrate. Cas9 DSB sites for the engineered gRNA target (outer cuts) are marked by blue vertical arrows. The two genomic gRNA targets in dRT are disabled by mutating PAM sites (shaded green). FIG. 12D is an illustration of exon 20 and the 5′ and 3′ genomic intron regions where the genomic gRNA target sites (solid triangles) are located. The genomic sequence with exon20 (solid parallel black lines) was PCR-produced, cloned into a vector and used for in-vitro validation of the gRNA target sites for Cas9 cleavage. FIG. 12E is an agarose gel showing results of the in vitro Cas9 assay. The 321 bp fragment in the vector was cleaved by the active sgRNA/Cas9 enzyme complex in Left Lane, resulting in 3 bands (3075 bp, 321 bp & 182 bp; black arrows) with NcoI restriction enzyme in the assay. Middle lane shows the uncleaved vector. Right lane is a DNA size ladder. FIG. 12F is a map of the 5077 bp iCAP plasmid containing DNA replacement template (donor construct) for replacing mutant MED13L exon20. Position of Cas9 target sites, overhang substrates, hPGK promoter-puromycin resistant gene, wildtype exon20, disabled genomic gRNA target sites (diamond flag), and vector backbone sequences retained in dRT (short red lines) are indicated in the map. SphI and SacI restriction enzyme digestions generate a 2145 bp dRT which was introduced into patient cell with sgRNA/Cas9 expression vector, and DSBs at engineered gRNA target sites by Cas9 create the 2026 bp edited isogenic fragment as the “patching” repair template. 100 bp overhang substrate regions between engineered gRNA target sites and disable genomic gRNA target sites are regions that may undergo 5′-to-3′ end resection following DSB generation, and are homologous to the similarly resected regions within the endogenous allele. FIG. 12G is an agarose gel showing the results of an in vitro Cas9 assay for validation of engineered gRNA targets. The 2145 bp SphI-SacI dRT was cleaved by the active sgRNA/Cas9 enzyme complex in Middle Lane resulting in three fragments (2026 bp, 67 bp and 52; black arrows). Right lane contained a single band of the same 2145 bp SphI-SacI dRT mixed with Cas9 but no sgRNAs specific for the engineered gRNA targets, indicating no cleavage occurred. Left Lane contained a DNA size marker.

FIG. 13A-13B are a diagram and images showing the results of genotypic analysis of iCAP edited mutant allele of MED13L exon20. FIG. 13A is a diagram showing the predicted structure of the successfully edited MED13L allele. Primers for genotyping the edited allele are indicated (“F” and “R” arrows). The expected size of positive PCR products is 1392 bp (primer For/Rev 1) and 1378 bp (primer For/Rev 2) for the edited region containing a replacement of mutant exon20 with the edited isogenic fragment which was pre-constructed to contain wildtype exon20 and a puromycin resistant gene. Vertical dashed lines indicate the limits of homology present in DNA the replacement template. FIG. 13B is agarose gels showing the correct size of PCR products amplified with genomic DNA templates extracted from the edited patient cells labeled as 4-1-2, indicating successful iCAP editing.

FIG. 14 is a diagram of sequencing data from analyzing PCR products of 1392 bp (For/Rev 1) and 1378 bp (For/Rev 2) generated from the edited allele as shown in the middle. Shown in the top sequence panel is the region spanning the 5′ paste site with the un-shaded sequence not present in dRT, the 5′ overhang substrate sequence framed in orange lines and partial 5′ hPGK promoter shaded in blue. Partial 3′ puromycin resistant gene at the stop codon TGA (in blue frame) and partial exon20 sequence (in black frame) with restored codon TCC for Serine at 1497 (F1497S) after the elimination of the single nucleotide base Thymine duplication by iCAP are shown in the middle sequence panel. Shown in the bottom sequence panel is the region spanning the 3′ paste site with the 3′ overhang substrate sequence framed in orange lines and the un-shaded sequence not present in dRT. Dashed vertical red lines are limits of overhang substrate sequences presented in dRT, dashed vertical black lines indicate locations of DSBs (inner cuts) on endogenous genomic sequences, diamond flags indicate mutated PAM, horizontal purple arrows are primers for PCR, and the red line boxed TCC nucleotides are the restored codon for Serine at 1497. The data indicate successful replacement of mutant exon20 with the wildtype exon20 of MED13L gene by iCAP editing through usage of Cas9.

FIGS. 15A-15D are diagrams showing the MED13L allele sequence in the edited region containing the edited isogenic fragment, which replaced a section of endogenous mutant exon20 and partial flanking intron sequences of MED13L gene by iCAP editing through usage of Cas9 as illustrated in FIG. 14. The sequence shown starts from the endogenous sequence upstream of the 5′ paste sites and ends in the endogenous sequence downstream of the 3′ paste site. Listed in FIG. 15D, shading indicates sequences corresponding to various features illustrated in previous figures.

FIGS. 16A-16F are diagrams illustrating the design of a proof of concept experiment involving editing the MED13L locus in the human genome using iCAP. FIG. 16A is an overview diagram of the study using iCAP (Design B) to eliminate the single nucleotide duplication from exon20 of MED13L mutant allele as illustrated in details in FIG. 12A. The exon20 region of human MED13L mutant allele (top), the DNA replacement template containing hPGK-Puromycin and wildtype exon 20 (middle), and the edited locus resulting from the iCAP process (bottom) are shown. FIG. 16B is an illustration of the endogenous MED13L genomic DNA sequences in the areas flanking the gRNA targeted sites on 5′ and 3′ sides of exon20 and the same (isogenic) genomic sequence areas in the pre-constructed DNA replacement template. The two genomic gRNA targets in the endogenous sequence are shown (arrowed “Cpf1 gRNA target” bars, top half the figure), and Cpf1 DSBs for each of the genomic gRNA targets are marked by vertical arrows. In bottom half of the figure, the same two genomic gRNA targets are also presented in dRT, and Cpf1 DSBs for each of the gRNA targets are marked by vertical arrows. There are no engineered gRNA target sites placed on the dRT. Cpf1 cleavages of dRT do not create overhang substrates sequences (mini-homology sequences) on both 5′ and 3′ ends of the edited isogenic fragment, and therefore, there is no homology sequences between the DSB ends, which are on the 5′ side of upstream DSB site and the 3′ side of downstream DSB site on genome, and the DSB ends on the edited isogenic fragment excised from dRT, except 5′ overhangs (shaded) created by Cpf1 cleavages at gRNA target sites. Shaded “ATTT” and “TTTA” sequences are PAM for Cpf1. FIG. 16C is an illustration of a vector containing exon 20 and the 5′ and 3′ genomic intron regions where the genomic gRNA target sites (solid triangles) are located. The distance between the two gRNA target sites is 417 bp. The genomic sequence with exon20 was cloned into a vector and used for in-vitro validation of the gRNA target sites for Cpf1 cleavage. FIG. 16D is an agarose gel showing results of the in vitro Cpf1 assay. The 417 bp fragment in the vector was cleaved and excised by the active sgRNA/Cpf1 enzyme complex in Left Lane, resulting in 2 bands (3161 bp & 417 bp; black arrows). Right lane shows the uncleaved vector. Middle lane is a DNA size ladder. FIG. 16E is a map of the 5071 bp iCAP plasmid containing DNA replacement template (donor construct) for replacing mutant exon20 of MED13L gene. Position of Cpf1 target sites (solid triangles), hPGK promoter-puromycin resistant gene (arrowed dark stripe), wildtype exon20 (black box), intron sequences (gray boxes) and vector backbone sequences (short dark lines) retained in dRT are shown. SphI and SacI restriction enzyme digestions generate a 2139 bp dRT being transfected into patient cells with sgRNA/Cpf1 expression vector, and DSBs at genomic gRNA target sites on dRT by Cpf1 will create the 1920 bp edited isogenic fragment as a “patching” repair template. FIG. 16F is an agarose gel showing the results of an in vitro Cpf1 assay for validation of the genomic gRNA targets on dRT. The 2139 bp SphI-SacI dRT was cleaved by the active sgRNA/Cpf1 enzyme complex in Left Lane, resulting in three fragments (1920 bp, 115 bp and 104 bp; black arrows) as expected. Middle lane contained the same 2139 bp SphI-SacI dRT mixed with Cpf1 but no sgRNAs specific for the gRNA, resulting in an intact SphI-SacI dRT (no cleavage). Right Lane contained a DNA size marker.

FIG. 17A-17B are a diagram and images showing the results of genotypic analysis of the iCAP edited MED13L mutant allele. FIG. 17A is a diagram showing the predicted structure of the successfully edited MED13L allele. Primers for genotyping the edited allele by PCR are indicated as purple arrows. The expected size of positive PCR products is approximately 1392 bp (primer For/Rev 1) and 1378 bp (primer For/Rev 2) for the edited region by the iCAP replacement of mutant exon20 with the edited isogenic fragment which is pre-constructed to contain wildtype exon20 and a puromycin resistant gene. Vertical dashed lines indicate the limits of endogenous sequence presented in the edited isogenic fragment after excision from dRT and are also the locations of DSBs by Cpf1 on genome. FIG. 17B is an agarose gel showing the expected sizes of PCR products (indicated by black arrows) amplified from genomic DNAs extracted from the edited patient cell populations labeled as 5-1-2 (right lanes in both top and bottom images), indicating successful iCAP editing. Right Lanes contained a DNA size marker.

FIG. 18 is a diagram of sequencing data from analyzing PCR products of approximate 1392 bp (For/Rev 1) and 1378 bp (For/Rev 2) generated from the edited allele as shown in the map (middle). Shown in the top sequence panel is the region of 5′ paste site; un-shaded sequence is partial intron not present in the edited isogenic fragment excised from dRT, sequence shaded in gray is the partial intron included in the edited isogenic fragment excised from dRT and “hPGK promoter”-shaded sequence is partial 5′ hPGK promoter sequence. Shown in the middle sequence panels, sequence framed in blue is partial 3′ puromycin resistant gene at the stop codon TGA and sequence framed in black is partial 3′ exon20 sequence showing an elimination of the single nucleotide base Thymine duplication and restored codon TCC for Serine at 1497 (mutation correction F1947S). The bottom sequence panel shows the region of 3′ paste site; gray shaded sequence is the part of intron included in the edited isogenic fragment excised from dRT and un-shaded sequence is the part of intron excluded from the edited isogenic fragment excised from dRT. Vertical dashed lines indicate locations of DSBs by Cpf at genomic gRNA target sites (5′ and 3′ paste sites) on genome and are also the limits of 5′ and 3′ ends of the edited isogenic fragment excised from dRT; horizontal “F” and “R” arrows are primers for PCR, and the red line boxed TCC nucleotides are the restored codon for Serine at 1497, black lined triangles indicate small deletions (less than 10 bp) at 5′ and 3′ paste sites which led to alterations of the originally genomic gRNA target sequences for the likely purpose of preventing further cleavage after end re-joining. The data indicate successful replacement of the mutant exon20 with the wildtype exon20 of MED13L gene by iCAP editing via usage of Cpf1.

FIG. 19A-19D are diagrams showing sequences of the edited MED13L mutant allele. The sequences shown starts from the 5′ endogenous genomic sequence, which was not present in the 5′ end of the edited isogenic fragment excised from dRT, through the entire edited isogenic fragment, which contains the wildtype exon20 and was pasted to replace the excised endogenous sequence of mutant exon20 following iCAP editing with usage of Cpf1, and ends at the 3′ endogenous genomic sequence which was not present in the 3′ end of the edited isogenic fragment excised from dRT. The restored codon TCC for Serine at amino acid position 1497 as a result of the iCAP replacement of mutant exon20 with a wildtype one is indicated by a box in FIG. 19C. Shading and coloring listed in FIG. 19D indicate sequences corresponding to various features illustrated in previous figures.

FIGS. 20A-20C are a diagram, an image showing the results of PCR genotyping and sequencing data of deletions of exon20 from MED13L locus. FIG. 20A is an illustration of the endogenous MED13L genomic DNA sequences in the areas of exon20 and flanking upstream and downstream introns, and gRNA target sites (arrowed “Cas9 gRNA target” bars for Cas9 and arrowed “Cpf1 gRNA target” bars for Cpf1) identified in the introns. “F” and “R” arrows are primers for PCR. Sequences shaded in light brown and grey are 5′ overhangs created by Cpf1 cleavage. Solid triangles indicate cleavages by Cas9 (blunt) and Cpf1 (5′ overhangs), respectively. FIG. 20B is an agarose gel showing the correct size of PCR products amplified from genomic DNAs extracted from the edited patient cells which were transfected with either sgRNA/Cas9 (iCAP Cas9) or sgRNAs/Cpf1 (iCAP Cpf1) expression vectors only and from the normal WI-38 human cell line, indicating successful deletion of exon20 by iCAP editing (iCAP deletion). A deletion of 321 bp by Cas9 cleavage at the two gRNA target sites resulted in a 759 bp PCR product while a deletion of 427 bp by Cpf1 cleavage at the two corresponding gRNA target sites led to a 653 bp PCR product. A 1080 bp PCR product reflects no deletion. FIG. 20C is a diagram of sequencing data from analyzing PCR products of 759 bp (Cas9) and 653 bp (Cpf1) generated from the alleles with the deletions in edited patient cell population. Sequence panels on tops of sequencing data show sequences flanking each DSBs at gRNA target sites. The line-crossed sequences between two vertical red dashed lines are the intervening endogenous sequences deleted, and solid red triangles indicate DSBs by Cas9 (blunt) and Cpf1 (5′ overhang). Solid vertical lines on sequencing data indicate where two DSB ends on genome without the intervening endogenous sequence were re-joined.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

The term, “polynucleotide” includes cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semisynthetic nucleotide bases. Also, included within the scope of the invention are alterations of a wild type or synthetic gene, including but not limited to deletion, insertion, substitution of one or more nucleotides, or fusion to other polynucleotide sequences.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” or “protein” are used interchangeably, and refer to a compound comprised of amino acid residues covalently linked by peptide bonds. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs and fusion proteins, among others. The polypeptides include natural peptides, recombinant peptides, synthetic peptides or a combination thereof. A peptide that is not cyclic will have a N-terminal and a C-terminal. The N-terminal will have an amino group, which may be free (i.e., as a NH₂ group) or appropriately protected (for example, with a BOC or a Fmoc group). The C-terminal will have a carboxylic group, which may be free (i.e., as a COOH group) or appropriately protected (for example, as a benzyl or a methyl ester). A cyclic peptide does not have free N- or C-terminal, since they are covalently bonded through an amide bond to form the cyclic structure. Amino acids may be represented by their full names (for example, leucine), 3-letter abbreviations (for example, Leu) and 1-letter abbreviations (for example, L). The structure of amino acids and their abbreviations may be found in the chemical literature, such as in Stryer, “Biochemistry”, 3rd Ed., W. H. Freeman and Co., New York, 1988. tLeu represents tert-leucine. neo-Trp represents 2-amino-3-(1H-indol-4-y)-propanoic acid. DAB is 2,4-diaminobutyric acid. Orn is ornithine. N-Me-Arg or N-methyl-Arg is 5-guanidino-2-(methylamino) pentanoic acid.

“Sample” or “biological sample” as used herein means a biological material from a subject, including but is not limited to organ, tissue, cell, exosome, blood, plasma, saliva, urine and other body fluid, A sample can be any source of material obtained from a subject.

The terms “subject”, “patient”, “individual”, and the like are used interchangeably herein, and refer to any animal, or cells thereof whether in vitro or in situ, amenable to the methods described herein. In certain non-limiting embodiments, the patient, subject or individual is a human. Non-human mammals include, for example, livestock and pets, such as ovine, bovine, porcine, canine, feline and murine mammals. Preferably, the subject is human. The term “subject” does not denote a particular age or sex.

The term “measuring” according to the present invention relates to determining the amount or concentration, preferably semi-quantitatively or quantitatively. Measuring can be done directly.

As used herein the term “amount” refers to the abundance or quantity of a constituent in a mixture.

The term “concentration” refers to the abundance of a constituent divided by the total volume of a mixture. The term concentration can be applied to any kind of chemical mixture, but most frequently it refers to solutes and solvents in solutions.

As used herein, the terms “reference”, or “threshold” are used interchangeably, and refer to a value that is used as a constant and unchanging standard of comparison.

As used herein, “paired-end sequencing” is a sequencing method that is based on high throughput sequencing, particular based on the platforms currently sold by Illumina and Roche. Illumina has released a hardware module (the PE Module) which can be installed in an existing sequencer as an upgrade, which allows sequencing of both ends of the template, thereby generating paired end reads. Paired end sequencing may also be conducted using Solexa technology in the methods according to the current invention. Examples of paired end sequencing are described for instance in US20060292611 and in publications from Roche (454 sequencing).

As used herein the term “sequencing” refers to determining the order of nucleotides (base sequences) in a nucleic acid sample, e.g. DNA or RNA. Many techniques are available such as Sanger sequencing and high-throughput sequencing technologies (also known as next-generation sequencing technologies) such as the GS FLX platform offered by Roche Applied Science, based on pyrosequencing.

A “restriction endonuclease” or “restriction enzyme” refers to an enzyme that recognizes a specific nucleotide sequence (target site) in a double-stranded DNA molecule, and will cleave both strands of the DNA molecule at or near every target site, leaving a blunt or a staggered end.

A “Type-IIs” restriction endonuclease refers to an endonuclease that has a recognition sequence that is distant from the restriction site. In other words, Type IIs restriction endonucleases cleave outside of the recognition sequence to one side. Examples thereof are NmeAlll (GCCGAG(21/19)) and FokI, AlwI, Mme I. Also included in this definition are Type IIs enzymes that cut outside the recognition sequence at both sides.

A “Type IIb” restriction endonuclease cleaves DNA at both sides of the recognition sequence.

“Restriction fragments” or “DNA fragments” refer to DNA molecules produced by digestion of DNA with a restriction endonuclease are referred to as restriction fragments. Any given genome (or nucleic acid, regardless of its origin) can be digested by a particular restriction endonuclease into a discrete set of restriction fragments. The DNA fragments that result from restriction endonuclease cleavage can be further used in a variety of techniques and can, for instance, be detected by gel electrophoresis or sequencing. Restriction fragments can be blunt ended or have an overhang. The overhang can be removed using a technique described as polishing. The term ‘internal sequence’ of a restriction fragment is typically used to indicate that the origin of the part of the restriction fragment resides in the sample genome, i.e. does not form part of an adapter. The internal sequence is directly derived from the sample genome, its sequence is hence part of the sequence of the genome under investigation.

The term “transposon” or “transposable element (TE)” or “retrotransposon” refers to a DNA sequence that can change its position within the genome, sometimes creating or reversing mutations and altering the cell's genome size. Transposition often results in duplication of the TE. Transposable elements (TEs) represent one of several types of mobile genetic elements. TEs are assigned to one of two classes according to their mechanism of transposition, which can be described as either copy and paste (class I TEs) or cut and paste (class II TEs). Class I TEs are copied in two stages: first they are transcribed from DNA to RNA, and the RNA produced is then reverse transcribed to DNA. This copied DNA is then inserted at a new position into the genome. The reverse transcription step is catalyzed by a reverse transcriptase. The cut-and-paste transposition mechanism of class II TEs does not involve an RNA intermediate. The transpositions are catalyzed by several transposase enzymes. Some transposases non-specifically bind to any target site in DNA, whereas others bind to specific DNA sequence targets. The transposase makes a staggered cut at the target site resulting in single-strand 5′ or 3′ DNA overhangs (sticky ends). This step cuts out the DNA transposon, which is then ligated into a new target site; this process involves activity of a DNA polymerase that fills in gaps and of a DNA ligase that closes the sugar-phosphate backbone. This results in duplication of the target site.

As used herein, “Ligation” refers to the enzymatic reaction catalyzed by a ligase enzyme in which two double-stranded DNA molecules are covalently joined together is referred to as ligation. In general, both DNA strands are covalently joined together, but it is also possible to prevent the ligation of one of the two strands through chemical or enzymatic modification of one of the ends of the strands. In that case, the covalent joining will occur in only one of the two DNA strands.

“Adapters” are short double-stranded DNA molecules with a limited number of base pairs, e.g. about 10 to about 30 base pairs in length, which are designed such that they can be ligated to the ends of restriction fragments. Adapters are generally composed of two synthetic oligonucleotides that have nucleotide sequences which are partially complementary to each other. When mixing the two synthetic oligonucleotides in solution under appropriate conditions, they will anneal to each other forming a double-stranded structure. After annealing, one end of the adapter molecule is designed such that it is compatible with the end of a restriction fragment and can be ligated thereto; the other end of the adapter can be designed so that it cannot be ligated, but this need not be the case (double ligated adapters). Adapters can contain other functional features such as identifiers, recognition sequences for restriction enzymes, primer binding sections etc. When containing other functional features the length of the adapters may increase, but by combining functional features this may be controlled.

“Adapter-ligated restriction fragments” refer to restriction fragments that have been capped by adapters on one or both ends.

As used herein, “barcode” or “tag” refer to a short sequence that can be added or inserted to an adapter or a primer or included in its sequence or otherwise used as label to provide a unique barcode (aka barcode or index). Such a sequence barcode (tag) can be a unique base sequence of varying but defined length, typically from 4-16 bp used for identifying a specific nucleic acid sample. For instance 4 bp tags allow 4⁴=256 different tags. Using such an barcode, the origin of a PCR sample can be determined upon further processing or fragments can be related to a clone. Also clones in a pool can be distinguished from one another using these sequence based barcodes. Thus, barcodes can be sample specific, pool specific, clone specific, amplicon specific etc. In the case of combining processed products originating from different nucleic acid samples, the different nucleic acid samples are generally identified using different barcodes. Barcodes preferably differ from each other by at least two base pairs and preferably do not contain two identical consecutive bases to prevent misreads. The barcode function can sometimes be combined with other functionalities such as adapters or primers and can be located at any convenient position. A barcode is often used as a fingerprint for labeling a DNA fragment and/or a library and for constructing a multiplex library. The library includes, but not limited to, genomic DNA library, cDNA library and ChIP library. Libraries, of which each is separately labeled with a distinct barcode, may be pooled together to form a multiplex barcoded library for performing sequencing simultaneously, in which each barcode is sequenced together with its flanking tags located in the same construct and thereby serves as a fingerprint for the DNA fragment and/or library labeled by it. A “barcode” is positioned in between two restriction enzyme (RE) recognition sequences. A barcode may be virtual, in which case the two RE recognition sites themselves become a barcode. Preferably, a barcode is made with a specific nucleotide sequence having 0 (i.e., a virtual sequence), 1, 2, 3, 4, 5, 6, or more base pairs in length. The length of a barcode may be increased along with the maximum sequencing length of a sequencer.

As used herein, “primers” refer to DNA strands which can prime the synthesis of DNA. DNA polymerase cannot synthesize DNA de novo without primers: it can only extend an existing DNA strand in a reaction in which the complementary strand is used as a template to direct the order of nucleotides to be assembled. The synthetic oligonucleotide molecules which are used in a polymerase chain reaction (PCR) as primers are referred to as “primers”.

As used herein, the term “DNA amplification” will be typically used to denote the in vitro synthesis of double-stranded DNA molecules using PCR. It is noted that other amplification methods exist and they may be used in the present invention without departing from the gist.

As used herein, “aligning” means the comparison of two or more nucleotide sequences based on the presence of short or long stretches of identical or similar nucleotides. Several methods for alignment of nucleotide sequences are known in the art, as will be further explained below.

“Alignment” refers to the positioning of multiple sequences in a tabular presentation to maximize the possibility for obtaining regions of sequence identity across the various sequences in the alignment, e.g. by introducing gaps. Several methods for alignment of nucleotide sequences are known in the art, as will be further explained below.

The term “isogenic” as used herein refers to sections of nucleotide sequence which are identical on separate DNA molecules or sections of the same larger DNA molecules. For example, the homologous flanking sequences of a replacement template of the current invention can be isogenic or identical to the corresponding sequences of the target locus in the genomic DNA.

The term “contig” is used in connection with DNA sequence analysis, and refers to assembled contiguous stretches of DNA derived from two or more DNA fragments having contiguous nucleotide sequences. Thus, a contig is a set of overlapping DNA fragments that provides a partial contiguous sequence of a genome. A “scaffold” is defined as a series of contigs that are in the correct order, but are not connected in one continuous sequence, i.e. contain gaps. Contig maps also represent the structure of contiguous regions of a genome by specifying overlap relationships among a set of clones. For example, the term “contigs” encompasses a series of cloning vectors which are ordered in such a way as to have each sequence overlap that of its neighbors. The linked clones can then be grouped into contigs, either manually or, preferably, using appropriate computer programs such as FPC, PHRAP, CAP3 etc.

“Fragmentation” refers to a technique used to fragment DNA into smaller fragments. Fragmentation can be enzymatic, chemical or physical. Random fragmentation is a technique that provides fragments with a length that is independent of their sequence. Typically, shearing or nebulisation are techniques that provide random fragments of DNA. Typically, the intensity or time of the random fragmentation is determinative for the average length of the fragments. Following fragmentation, a size selection can be performed to select the desired size range of the fragments

“Physical mapping” describes techniques using molecular biology techniques such as hybridisation analysis, PCR and sequencing to examine DNA molecules directly in order to construct maps showing the positions of sequence features.

“Genetic mapping” is based on the use of genetic techniques such as pedigree analysis to construct maps showing the positions of sequence features on a genome

The term “genome”, as used herein, relates to a material or mixture of materials, containing genetic material from an organism. The term “genomic DNA” as used herein refers to deoxyribonucleic acids that are obtained from an organism. The terms “genome” and “genomic DNA” encompass genetic material that may have undergone amplification, purification, or fragmentation.

The term “reference genome”, as used herein, refers to a sample comprising genomic DNA to which a test sample may be compared. In certain cases, reference genome contains regions of known sequence information.

The term “double-stranded” as used herein refers to nucleic acids formed by hybridization of two single strands of nucleic acids containing complementary sequences. In most cases, genomic DNA are double-stranded.

As used herein, the term “single nucleotide polymorphism”, or “SNP” for short, refers to single nucleotide position in a genomic sequence for which two or more alternative alleles are present at appreciable frequency (e.g., at least 1%) in a population.

The term “chromosomal region” or “chromosomal segment”, as used herein, denotes a contiguous length of nucleotides in a genome of an organism. A chromosomal region may be in the range of 1000 nucleotides in length to an entire chromosome, e.g., 100 kb to 10 MB for example.

The terms “sequence alteration” or “sequence variation”, as used herein, refer to a difference in nucleic acid sequence between a test sample and a reference sample that may vary over a range of 1 to 10 bases, 10 to 100 bases, 100 to 100 kb, or 100 kb to 10 MB. Sequence alteration may include single nucleotide polymorphism and genetic mutations relative to wild-type. In certain embodiments, sequence alteration results from one or more parts of a chromosome being rearranged within a single chromosome or between chromosomes relative to a reference. In certain cases, a sequence alteration may reflect a difference, e.g. abnormality, in chromosome structure, such as an inversion, a deletion, an insertion or a translocation relative to a reference chromosome, for example.

As used herein, the term “endonuclease” refers to a family of enzymes that has an activity described as EC 3.1.21, EC 3.1.22, or EC 3.1.25, according to the IUBMB enzyme nomenclature. Site-specific endonucleases recognize specific nucleotide sequences in double-stranded DNA. Some sequence-specific endonucleases cleave only one of the strands in a duplex and are referred to herein as “nicking endonucleases”. Nicking endonuclease catalyzes the hydrolysis of a phosphodiester bond, resulting in either a 5′ or 3′ phosphomonoester.

A “site-specific nicking endonuclease”, as used herein, denotes a nicking endonuclease that cleaves one strand of a double-stranded nucleic acid by recognizing a specific sequence on the nucleic acid. The cleavage site or “nick site” of the phosphodiester backbone may fall within or immediately adjacent the recognition sequence of the site-specific nicking endonuclease.

The terms “edited fragment”, “edited isogenic fragment”, “edited DNA fragment”, “edited isogenic DNA fragment”, “repair fragment”, “patching repair template”, “donor fragment”, “donor construct”, “DNA donor”, and “inserted construct” are interchangeable terms used herein to describe a polynucleotide molecule which is pre-constructed in vitro and used to replace the endogenous, homologous section of genomic DNA after it is cleaved from a DNA replacement template. The edited DNA fragment contains the identical endogenous DNA sequences with altered nucleotide compositions, and the edited DNA fragment can additionally includes overhang substrate sequences that are the endogenous DNA sequences adjacent to 5′ and 3′ ends of the excised DNA section of genome and placed on both of the 5′ and 3′ ends of the edited fragment.

The term “overhang substrates”, “homology arms”, and “homology regions” are interchangeable terms used herein to refer to homologous DNA sequences flanking the target DNA sequence in both the repair template and genomic DNA. Overhang substrates are sites of 5′ to 3′ resection, which provide “sticky end” overhangs that enable the edited fragment to bind and anneal to the genomic DNA. The nucleotide length and sequence of overhang substrates required for iCAP is flexible and can be adapted to achieve the specificity required to edit a particular target DNA sequence.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2, 7, 3, 4, 5, 5.3, and 6. This applies regardless of the breadth of the range.

### DESCRIPTION

The present invention provides methods editing, mutating, or modifying a genomic target DNA sequences in a cell using the “in situ cut and paste” or iCAP method. The iCAP method enables excising (the “cut”) a section of DNA sequence (referred to as genomic target sequence or genomic target DNA sequence) of a genome and patching (the “paste”) the lost section with an edited DNA fragment released from a DNA replacement template (dRT) in the target sequence's natural location (in-situ), a scheme heretofore only achievable for recombinant DNA in the test tube (in-vitro) using restriction enzymes. The edited DNA fragment (1) contains altered nucleotide compositions in the identical endogenous DNA sequences as those to be excised from a genome, (2) may or may not include overhang substrate sequences that are the extra endogenous DNA sequences adjacent to 5′ and 3′ ends of the excised DNA section of genome and presented (placed) on both of the 5′ and 3′ ends of the edited fragment, (3) resides in the dRT, which is pre-constructed in-vitro prior to the actual genome editing in cells or zygotes, and (4) is released from the dRT by programmable nucleases' cleavage occurring only inside cells or zygotes. The edited DNA fragment is also referred to as the edited isogenic fragment.

The iCAP method is based on the concept that when DNA cleavage (double strand break, DSB) occurs at two sites within a genome, the intervening sequence between the two cleavage sites is excised. The same scenario will also occur to dRT which contains (1) the same intervening sequence with precisely altered nucleotide compositions (called an edited isogenic fragment) and (2) either the same two cleavage sites as those present on genome or unrelated and unique designed cleavage sites flanking the edited isogenic fragment. This process allows the edited fragment to be excised from dRT by cleavage occurring within cells or zygotes where the genomic target sequence resides. When such DNA cleavages occur to both genome and dRT in situ, the edited isogenic fragment excised from dRT can patch (replace) the lost endogenous intervening sequence by re-joining the fragment at the two cleavage sites on genome, resulting in the altered nucleotide compositions incorporated into the precise locations of a genome.

The iCAP process achieves precise genome editing through a coordinated selection or design of two cleavage (DSB) sites (a 5′ and a 3′ locations) in the genome and on the dRT, respectively. Using two cleavage sites allows Crispr/Cas programmable nucleases, (i.e. Cas9, Cas12a (Cpf1), among others) to simultaneously cut and excise (1) a section of the endogenous DNA sequence, within which nucleotide alterations are to be made, from genome and (2) an edited isogenic fragment, constructed beforehand to contain those alterations, from dRT. The excision step is followed by re-joining the edited fragment at upstream (5′) and downstream (3′) cleavage sites in the genomic DNA to replace the lost section of the endogenous sequence between the two DSBs. The re-joining step is mediated by endogenous DNA repair pathways of either classic non-homologous end joining (c-NHEJ) or end-resection associated homology directed repair (ER-HDR) or a combination of both. The ER-HDR may dominantly mediate the re-joining if extra DNA sequences, which flank the 5′ side of the upstream cleavage site and the 3′ side of the downstream cleavage site in genome, respectively, are also included and presented at the 5′ and 3′ ends of an edited isogenic fragment in dRT (Design A). The extra DNA sequences included in the edited fragment serve as overhang substrate sequences which are homology to those flanking the cleavage sites of genome. The overhang substrate sequences, which are also called mini homology sequences if less than 100 bp, provide sequence zones for 5′→3′ end-resection to create single stranded 3′ overhangs which will be complementary between cleavage ends of genome and the ends of the an edited isogenic fragment. In contrast, when the same 5′ as well as the same 3′ gRNA target sites presented on both genome and dRT (Design B) are selected as cleavage sites, the re-joining is most likely mediated by c-NHEJ to bridge the broken genome with the edited isogenic fragment, which bears no overhang substrate sequences, at the corresponding 5′ and 3′ cleavage ends.

In Design A, through a specific and coordinated design, an uniquely engineered gRNA cleavage recognition site, which is completely different from those for excising endogenous DNA sequence, is incorporated in dRT to allow excision of an edited fragment bearing overhang substrate sequences (mini-homology sequences). This design utilizes ER-HDR to allow (1) using Crispr/Cas programmable nucleases, i.e. Cas9, to simultaneously make blunt-ended cuts to excise a section of endogenous DNA sequence from genome as well as an edited fragment from dRT in-situ, resulting in DNA damages (DSB) at these excision sites; (2) the 5′→3′ resection, which is the initial steps to repair damaged DNA at DSB ends of genome, may also take place at DSB ends of the edited fragment excised from dRT (or alternatively to say, takes place not only at the damaged ends of genome but also at the DSB ends of an edited fragment released from dRT); (3) the 5′→3′ resection in the overhang substrate sequences flanking the DSB ends results in formations of complementary 3′ overhang sequences at the broken ends of genome and the edited fragment; (4) the edited fragment patches (paste) the broken genome by annealing the complementary 3′ overhang sequences at both of the upstream and downstream DSB sites and (5) DNA synthesis and ligation at the annealing sites complete the repairs, leading to the genome edited as a result of the replacement of the original section of endogenous DNA sequence with the edited fragment which is in-vitro pre-constructed to contain altered nucleotide compositions.

In Design B, there will be no overhang substrate sequences presented and flanking the edited isogenic fragment in dRT. Therefore, the 5′ side and 3′ side sequences flanking the 5′ (upstream) and 3′ cleavage sites (downstream), respectively, on genome, are not homology to those on the 5′ and 3′ ends of the edited fragment following cut and excision occurred to genome and dRT. The iCAP precise genome editing by this design likely utilizes either c-NHEJ or alternative-NHEJ (alt-NHEJ, one of ER-HDR pathways) to re-join the 5′ and 3′ DSB ends between genome and the edited fragment, respectively. The re-joining can also be mediated by c-NHEJ at one DSB end (5′ or 3′ cleavage site) and by alt-NHEJ at the other DSB end (3′ or 5′ cleavage site). The Design B was exemplified by editing human mutant MED13L gene allele using the programmable nuclease Cas12a (Cpf1).

The iCAP process also allows a precise deletion of a section of endogenous genome sequences between two gRNA target sites cleaved by programmable nucleases such as Cas9 and Cpf1 (Cas12a), resulting in a flawless end re-joining of the broken genome without the intervening sequence between the two target sites, regardless if a dRT is present or not. To differentiate the iCAP process which leads to a replacement of a section of endogenous sequences with an edited fragment (iCAP replacement or iCAP-r), the ‘perfect’ deletion of a section of endogenous genome sequence achieved by iCAP is called iCAP deletion or iCAP removal (iCAP-d or iCAP-r).

### Cas and Cpf1 Nucleases

In some embodiments, the invention includes a method of editing, mutating, or modifying a genomic target DNA sequence in a cell, the method comprising: providing (i) a DNA replacement template (dRT) comprising the target DNA sequence comprising the desired edited, mutated, or modified nucleotide(s), and (ii) a sequence encoding a nuclease; contacting the genomic target DNA sequence, the DNA RT, and heterologous guide-RNAs (gRNAs) under conditions that allow for the gRNAs to induce double-strand breaks of the genomic target DNA sequence and the DNA RT by the nuclease; subjecting the blunt ends at excision sites of genome and DNA RT to 5′→3′ DNA end resection to generate complementary 3′ overhangs; annealing 3′ complementary overhangs of the edited fragment released from dRT to the complementary 3′ overhang sequences at excision sites of genome; and ligating the annealing sites, thereby resulting in incorporation of the edited fragment in the place of the genomic target DNA sequence. In some embodiments, the nuclease is a Cas9 or Cpf1 nuclease or a natural or engineered variant thereof. One who is skilled in the art would recognize the advantages and features of the various nucleases, and would be able to identify the variant most suitable for the application of the iCAP method of the current invention.

Cas9 nucleases, or CRISPR-associated protein 9 (formerly called Cas5, Csn1, or Csx12) is a 160 kDa dual RNA-guided DNA endonuclease that catalyzes site-specific cleavage of double-stranded DNA. Cas9 was originally discovered as a key component in the Clustered Regularly Interspaced Short Palindromic Repeats (CRISPR) system, a form of adaptive immune system in Streptococcus pyogenes. The bacterial immune system uses Cas9 to monitor and degrade foreign DNA from invading bacteriophages or plasmids. Cas9 exists as a complex of a nuclease enzyme protein and a guide RNA or gRNA molecule, which confers target DNA sequence specificity. Cas9 is able to detect foreign DNA molecules by unwinding the target DNA to expose any base sequences what are complementary to a 20 base pair spacer region of the guide RNA. If the target molecule is complementary to the guide RNA and associated with a PAM (protospacer adjacent motif) site, the nuclease activity of Cas9 is activated, resulting in cleavage of the invading DNA. Studies of CRISPR-like systems in other bacteria have identified a number of naturally-occurring variants of Cas9 which can be adapted for use in DNA-editing systems, such as the iCAP system of the current invention. Examples of naturally-occurring Cas9 enzymes include, but are not limited to SpCas9, SaCas9, StCas9, NmCas9, FnCas9, CjCas9, CasX, CasY, Cas12a, Cas14a, BlCas9, ScCas9, LmoCas9, TdCas9, Nme2Cas9, GsCas9, BlatCas9, and FnCas9-RHA. In some embodiments, the Cas12a may be one of the natural variants known to the art, which include but are not limited to AsCpf1, FnCpf1, LbCpf1, AsCpf1-RR, LbCpf1-RR, AsCpf1-RVR. In some embodiments, the invention includes a naturally occurring Cas nuclease variant. Examples of Cas-variant nucleases include but are not limited to Cas13a/b(C2c2), Cas12b(C2c1), Cas12c(C2c3). In some embodiments, the iCAP method of the current invention comprises the use of a naturally-occurring Cas9 nuclease.

The popularity of Cas9 in molecular biology applications has led to the development of modified or engineered versions of the Cas9 nuclease which provide improved function or specificity, depending on the desired application including but not limited to reducing off-target effects and modifying the rate of reaction. In certain embodiments of the current invention, the Cas9 nuclease is an engineered variant thereof. Examples of engineered Cas9 nucleases include, but are not limited to eSpCas9, SpCas9-HF1, Fok1-Fused dCas9, xCas9, SpCas9-VQR, SpCas9-VRER, SpCas9-D1135E, SpCas9-EQR, SpCas9-QQR1, Cas9-DD, HypaCas9, evoCas9, xCas9-3.7, SniperCas9, Cas9-CtIP, SpCas9-NG, Split-SpCas9, SpCas9-K855A, ScCas9+, ScCas9++, SaCas9-KKH, and SaCas9 among others. In certain embodiments of the invention, other endonucleases may also be used, including but not limited to Cpf1, T7, Cas3, Cas8a, Cas8b, Cas10d, Cse1, Csy1, Csn2, Cas4, Cas10, Csm2, Cmr5, Fok1, other nucleases known in the art, and any combination thereof.

Cpf1 or CRISPR from Prevotella and Francisella 1, which is also known as Cas12a in the art is a nuclease similar to Cas9 and can similarly be used in DNA editing methods, including those of the current invention. Cpf1 often offers certain advantages over Cas9 in DNA editing systems. Cpf1 endonuclease is smaller in size compared to Cas9 and requires shorter a CRISPR RNA (crRNA) to work properly. Cpf1 does not require a trans-activating crRNA (tracrRNA) while processing Cpf1-associated CRISPR repeats into mature crRNAs. Naturally-occurring variants or orthologs of Cpf1 nucleases from various bacteria have been isolated and assessed for genome editing, including AsCpf1 and LbCpf1, which were isolated from Acidaminococcus sp. BV3L6 and Lachnospiraceae bacterium ND2006, respectively, and are commonly used in DNA editing systems known in the art. The main advantage of a CRISPR/Cpf1-mediated genome-editing tool is the reengineering of the desired DNA as the target and that the PAM sequence (5′-TTTN-3′) remains intact.

### Cas Nuclease Programming

Cas and Cpf1 nuclease-based DNA editing systems such as those of the current invention are facile and efficient for inducing targeted genetic alterations. Target recognition by the nuclease enzyme requires a ‘seed’ sequence within the guide RNA (gRNA) and a conserved tri-nucleotide containing protospacer adjacent motif (PAM) sequence upstream of the gRNA-binding region. Cas and Cpf1 nucleases can thereby be engineered to cleave virtually any DNA sequence by redesigning the gRNA to be complementary to the target DNA sequence. The iCAP system of the current invention can simultaneously target multiple genomic loci by co-expressing a single Cas9 protein with two or more gRNAs, making this system uniquely suited for multiple gene editing or synergistic activation of target genes.

Cas and Cpf1-based gene editing occurs when a guide nucleic acid sequence specific for a target gene and a Cas endonuclease are introduced into a cell and form a complex that enables the Cas endonuclease to introduce a double strand break at the target gene and a replacement template DNA construct containing the desired sequence alteration or mutaiton. In certain embodiments, the iCAP system comprises one of more expression vectors. In other embodiments, the iCAP expression vector induces expression of Cas9 endonuclease. Other endonucleases may also be used, including but not limited to Cpf1, T7, Cas3, Cas8a, Cas8b, Cas10d, Cse1, Csy1, Csn2, Cas4, Cas10, Csm2, Cmr5, Fok1, other nucleases known in the art, and any combination thereof.

In certain embodiments, inducing the iCAP expression vector comprises exposing the cell to an agent that activates an inducible promoter in the Cas expression vector. In such embodiments, the iCAP expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

The guide nucleic acid sequence is specific for a gene and targets that gene for Cas or Cpf1 endonuclease-induced double strand breaks. The sequence of the guide nucleic acid sequence may be within a loci of the gene. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

The guide nucleic acid sequence may be specific for any gene, such as a gene that would reduce immunogenicity or reduce sensitivity to an immunosuppressive microenvironment. The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In some embodiments, the guide nucleic acid sequence comprises a single guide RNA.

In the context of formation of a gRNA/Cas9 complex, “target sequence” refers to a sequence to which a guide sequence is designed to have some complementarity, where hybridization between a target sequence and a guide sequence promotes the formation of a gRNA/Cas9 complex. Full complementarity is not necessarily required, provided there is sufficient complementarity to cause hybridization and promote formation of a gRNA/Cas9 complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous iCAP system, formation of a gRNA/Cas9 complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional. In certain embodiments, the tracr sequence has at least 50%, 60%, 70%, 80%, 90%, 95% or 99% of sequence complementarity along the length of the tracr mate sequence when optimally aligned. In other embodiments, one or more vectors driving expression of one or more elements of a iCAP system are introduced into a host cell, such that expression of the elements of the iCAP system direct formation of a iCAP complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the iCAP system not included in the first vector. iCAP system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a nuclease enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

The Cas and Cpf1 nucleases of the invention can be modified to increase nucleic acid binding affinity and/or specificity, alter an enzymatic activity, and/or change another property of the protein. In certain embodiments, the Cas and Cpf1 nucleases of the invention can be fusion proteins derived from a wild type Cas9 proteins or fragments thereof. In other embodiments, the nucleases can be derived from modified Cas9 proteins. For example, the amino acid sequence of the Cas9 protein can be modified to alter one or more properties (e.g., nuclease activity, affinity, stability, and so forth) of the protein. Alternatively, domains of the nuclease protein not involved in RNA-guided cleavage can be eliminated from the protein such that the modified nuclease protein is smaller than the wild type nuclease protein. In general, a Cas9 or Cpf1 protein comprises at least two nuclease (i.e., DNase) domains. For example, a Cas9 or Cpf1 protein can comprise a RuvC-like nuclease domain and a HNH-like nuclease domain. The RuvC and HNH domains work together to cut single strands to make a double-stranded break in DNA. (Jinek et al., 2012, Science, 337:816-821). In certain embodiments, the Cas9- or Cpf1-derived protein can be modified to contain only one functional nuclease domain (either a RuvC-like or a HNH-like nuclease domain). For example, the Cas9- or Cpf1-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

### Vectors

In one non-limiting embodiment, a vector drives the expression of the iCAP system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

### Introduction of Nucleic Acids

Methods of introducing nucleic acids into a cell include physical, biological and chemical methods. Physical methods for introducing a polynucleotide, such as DNA and RNA, into a host cell include calcium phosphate precipitation, lipofection, particle bombardment, microinjection, electroporation, and the like. DNA and RNA can be introduced into target cells using commercially available methods which include electroporation (Lonza 4D-Nucleofector, Amaxa Nucleofector-II, (Amaxa Biosystems, Cologne, Germany), ECM 830 (BTX) (Harvard Instruments, Boston, Mass.) or the Gene Pulser II (BioRad, Denver, Colo.), Multiporator (Eppendorf, Hamburg Germany). DNA and RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the target cells, transfecting the target cells, and electroporating the target cells. One nucleic acid may be introduced by one method and another nucleic acid may be introduced into the target cell by a different method.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in the experiments disclosed herein are now described.

### Mouse Genome Editing;

Analysis of gene loci, editing locations and identifications of gRNA target sites. Genomic DNA sequences of slc35F2 and slc35F6 were obtained from online resources, seast dot ensemble dot org/index dot html and ncbi dot nlm idot nih dot gov/pubmed/, and then catalogued & annotated using Snapgene software (Version 2.1.1) for further sequence analysis. Sequences of 500 nucleotides, flanking and spanning the editing locations, one at the intron 5′ of the last exon for FRT3 insertion and the other at the immediate 5′ side of a stop codon in the last exon for in-frame insertion of the expression cassette, were selected and analyzed for identifying gRNA target sites to induce “inner” cuts using the online tool “Benchling” (benchling dot com). Based on a category of programmable nuclease (Cas9, Cpf1, etc.), Benchling software analyzed the selected sequences for all available target sites and scores the sites according to established parameters and variables effecting on-target & off-target efficiencies. The gRNA target sites with favorable scores were chosen for validation assay in vitro, according to (1) a favorable combination of on-target and off-target scores (an on-target score>60 and an off-target score close to 100 are optimum) and (2) the close proximity to the desired site of insertion of any exogenous DNA sequence (for example, the endogenous STOP codon, if creation of an in-frame protein fusion is the goal.)

In vitro transcription of sgRNAs and validation of gRNA target sites. Templates for in vitro transcription of SpCas9 guides were amplified from the plasmid pX330 (Addgene, MA) using primer pairs with 65 oligonucleotides as a 5′ primer (forward) and the oligonucleotides AAAAGCACCGACTCGGTGCC (SEQ ID NO: 1) as a 3′ primer (reverse) The 65 oligonucleotides are comprised of GCGCGCTAATACGACTCACTATAGGNNNNNNNNNN NNNNNNNNNNGTTTTAGAGCTAGAAATAGC (SEQ ID NO: 2), in which NNN - - - represents 20 oligonucleotide protospacer proceeded with T7 minimal promoter. The protospacer corresponds to gRNA target sequences for inner cuts and to the engineered gRNA target sequence GTGCTTCGATATCGATCGTT (SEQ ID NO: 3) for outer cuts. The Phusion DNA Polymerase (NEB M0530S, neb dot com) was used for the amplification according to manufacturer's protocols, and amplified templates were purified with QIAquick PCR Purification Kit (Qiagen 28104, qiagen dot com). The in vitro transcription was performed using MEGAshortscript T7 Transcription Kit (LifeTechnologies AM1354, thermofisher dot com) following manufacturer's protocols. After incubation for 4 hours at 37° C., samples were treated with DNase I for 15 minutes at 37° C. to remove DNA templates. In vitro transcribed sgRNAs were purified and eluded with MEGAclear Purification Kit (LifeTechnologies AM1908M, thermofisher dot com) according to manufacturer's protocol, and the final concentration was measured using Nanodrop and was stored at −80° C. for subsequent uses. In assays to validate sgRNAs' on-target cut induced by programmable nuclease, 30 nM in vitro transcribed sgRNAs, 3 nM RNAse-free DNA fragments containing gRNA target sequences and 30 nM Cas9 protein (NEB Cas9 Nuclease M0386T, neb dot com) were mixed in reaction tubes as per manufacturer's protocol. At the end of the reaction, a 1 l RNAse A was added and then incubated for additional 15 minutes at 37° C. to degrade sgRNA. DNA fragments in the reaction were purified with QIAquick PCR Purification column (Qiagen 28104, qiagen dot com) to remove residual protein, followed by analysis in an agarose gel.

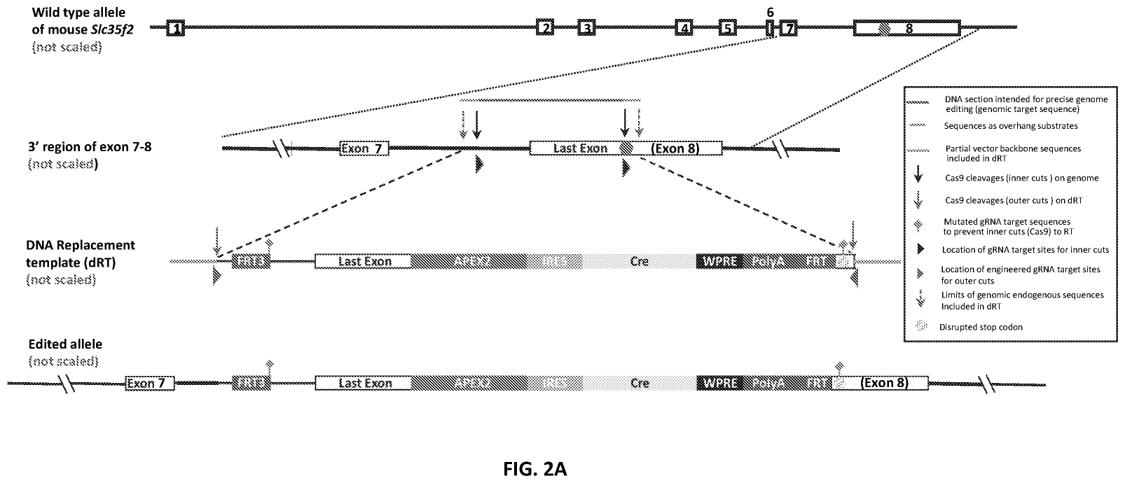

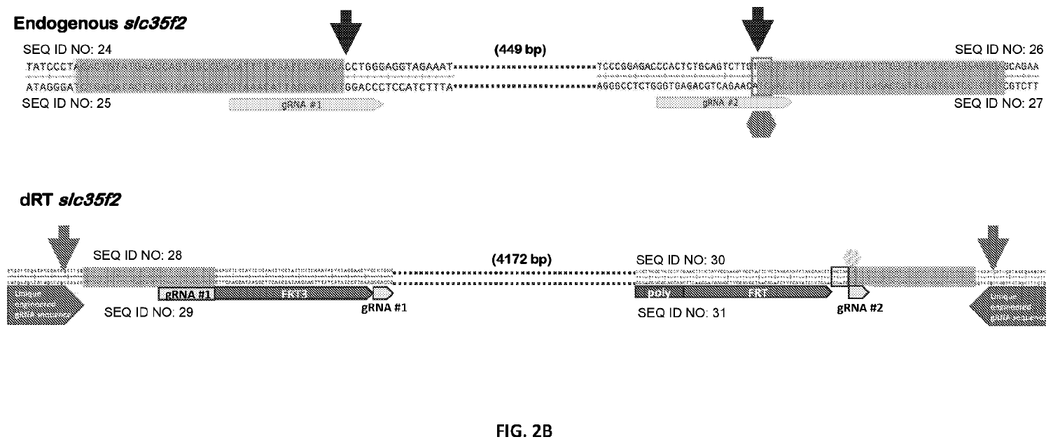

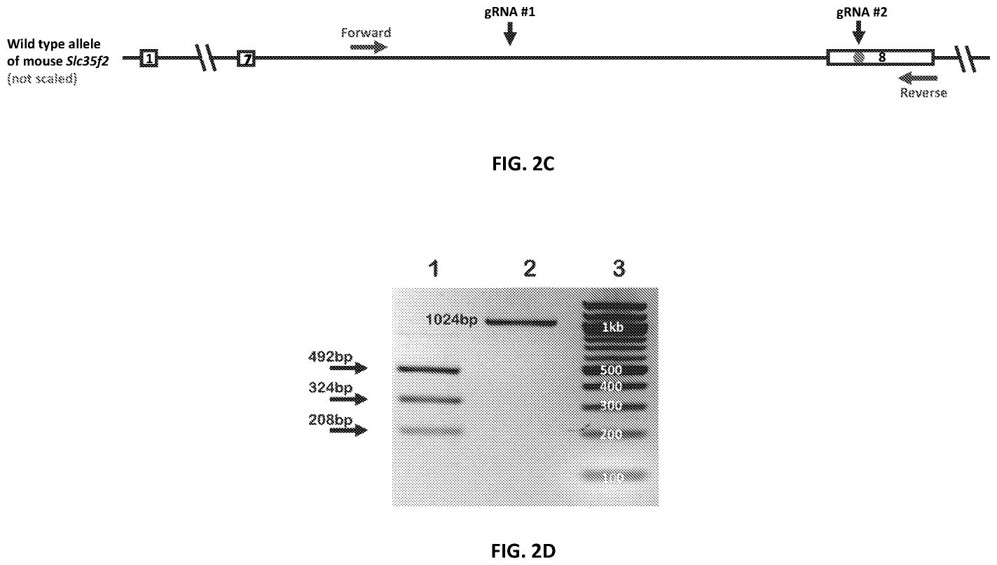

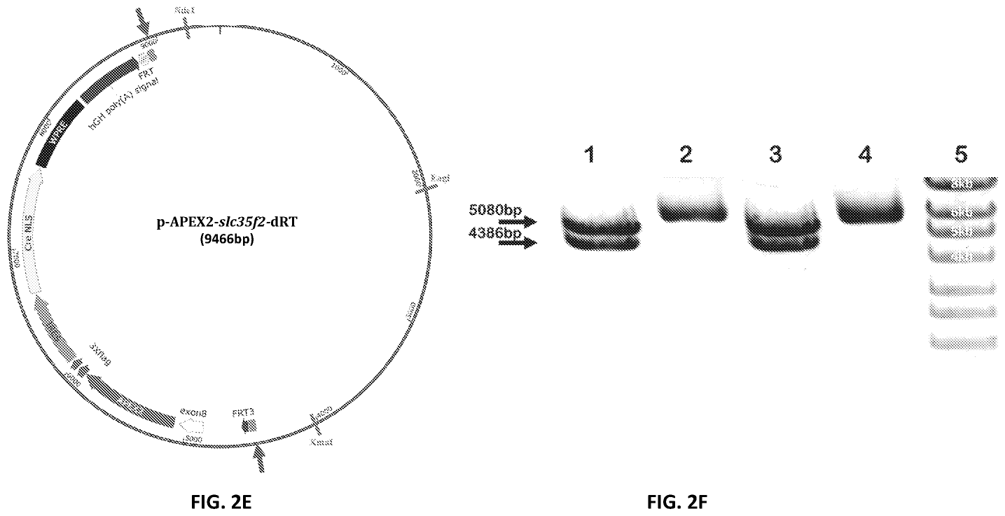

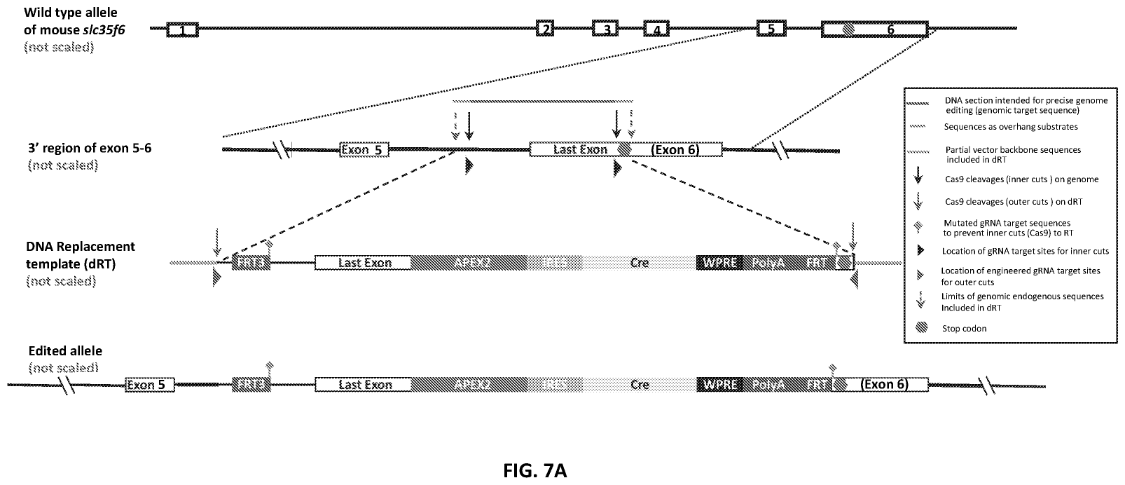

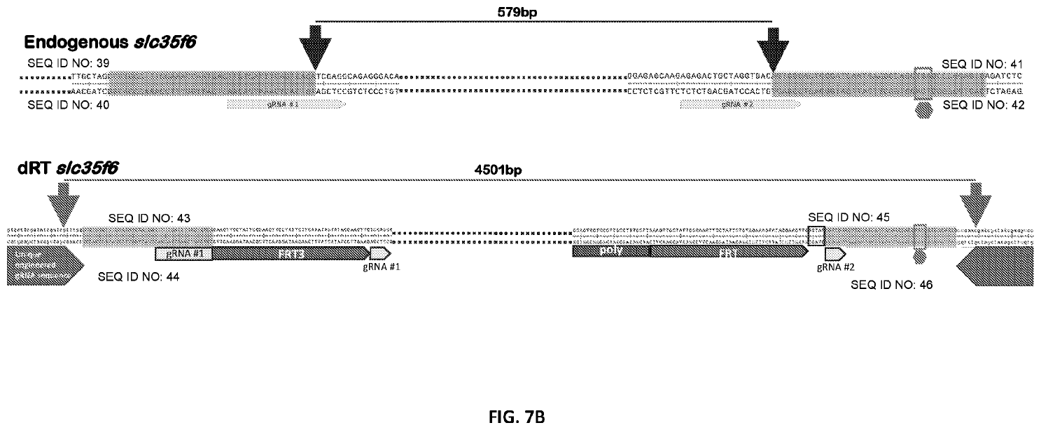

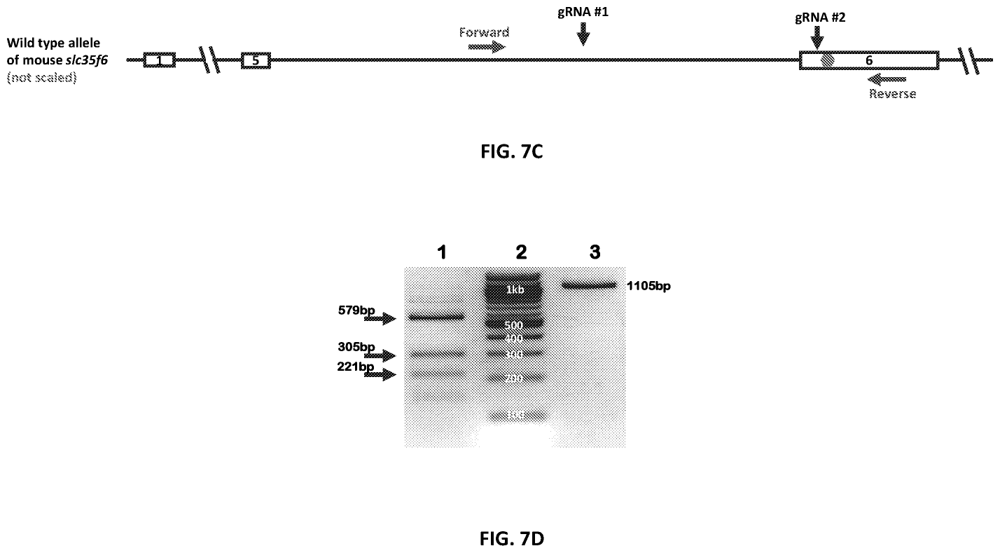

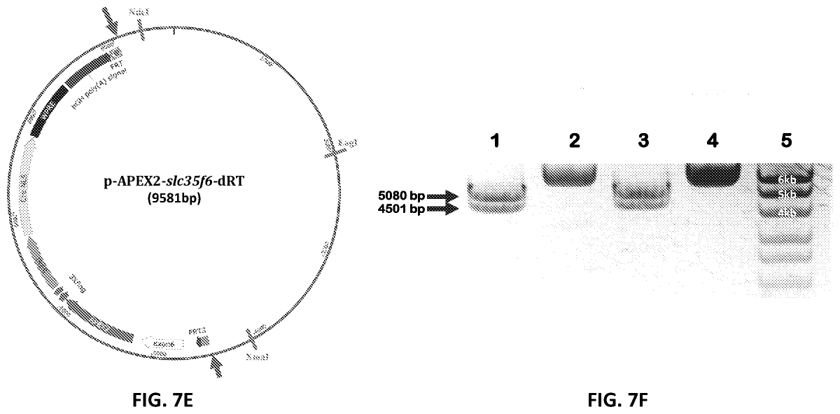

Construction of DNA replacement template containing edited nucleotide alterations. The intended editing was to make nucleotide alterations at two locations in gene loci of interests, (1) to insert a FRT3 sites in the intron 5′ of the last exon and (2) in-frame to insert a 3.7 expression cassette immediately 3′ side of the last codon of endogenous genes. Based on the analysis of gene loci, the editing locations and identification and validation of gRNA target sites, the edited fragment was designed in a pre-constructed replacement template to be organized in a 5′ to 3′ direction as 5′ overhang substrate sequence (40 bp intron sequence)-FRT3 (exogenous gene)-intron sequence-coding sequence of last exon-expression cassette ending with FRT-3′ overhang substrate sequence (40 bp sequence around and 3′ of the endogenous stop codon), as shown in FIGS. 2A and 7A. As the wild type genomic DNAs used for the construction of replacement templates contain gRNA target sequences for inner cuts, these target sequences were mutated by insertions of FRT3 and the expression cassette to disrupt these target sites to allow cuts only to be induced at outer cut sites on the replacement template, as shown in FIGS. 2B and 7B. The molecular construction of the DNA replacement template was, according to the organization, accomplished by using PCR, cloning, subcloning to assemble each of the components into a single edited fragment flanked with engineered gRNA target sequences (outer cut sites) in a vector as a final product. The entire edited fragment was sequenced and confirmed to have correct DNA sequences as designed. The replacement template containing the edited fragment flanked with partial vector backbone sequences was excised from the vector using restriction enzymes of NheI and XmaI before being introduced into zygotes.

Animal Production. The mice were contractively produced by the transgenic mouse facility at University of Pennsylvania School of Veterinary Medicine. The protocols and procedures for animal productions were approved by the Institutional Animal Care and Use Committee (IACUC). Briefly, a buffer solution mixed with sgRNAs (40-60 ng/μl), DNA replacement templates (1.5-2 ng/μl) and Cas9 mRNA (100 ng/μl, Trilink Biotechnologies, CA) was microinjected into pronuclei and cytoplasm of one-cell stage embryos obtained from superovulated B6D2F1 female mice (Jackson Laboratory, Maine). The injected embryos were maintained in M16 medium and cultured for at least one hour in a 100% humidified incubator with 5% C02 at 37° C. before implantation. A group of 20 injected embryos on average were transferred into oviducts of a pseudopregnant mouse for a full term development.

Isolation of genomic DNA from animals. Biopsies from ear were dissolved in 50 μl of Extracta DNA Prep Extraction Buffer (Quanta BioSciences 5091-025, quantabio dot com), and genomic DNAs were extracted following manufacturer protocol. Biopsies from tails were dissolved in lysis buffer (50 mM Tris-Cl, pH 8.0, 50 mM EDTA, 100 mM NaCl, 1% SDS, 0.2 mg/ml Proteinase K) and incubated for overnight at 55° C. with agitation, and then genomic DNAs were extracted by adding isopropanol to the dissolving buffer, in which tissue debris were prior eliminated, to obtain DNA precipitates. The extracted DNAs were resuspended in TE buffer after clean washes with ethanol.

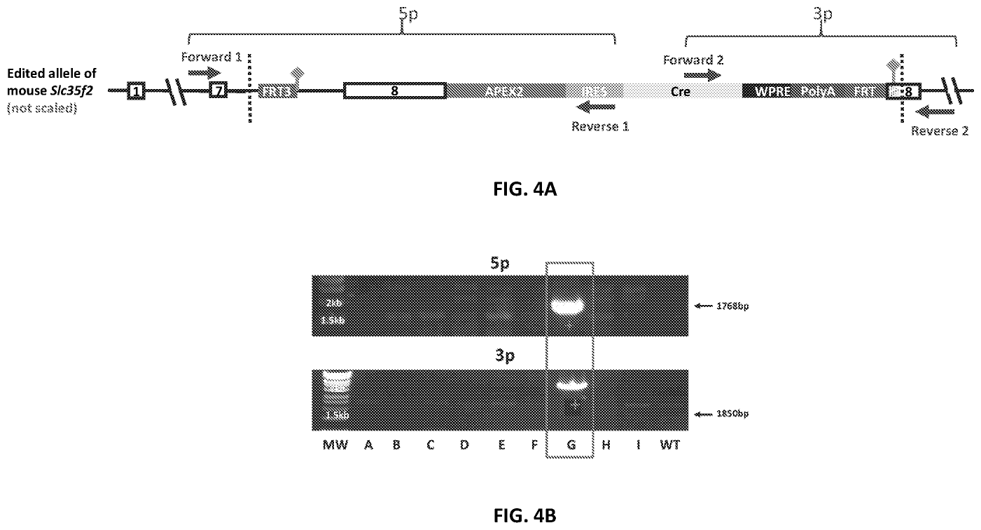

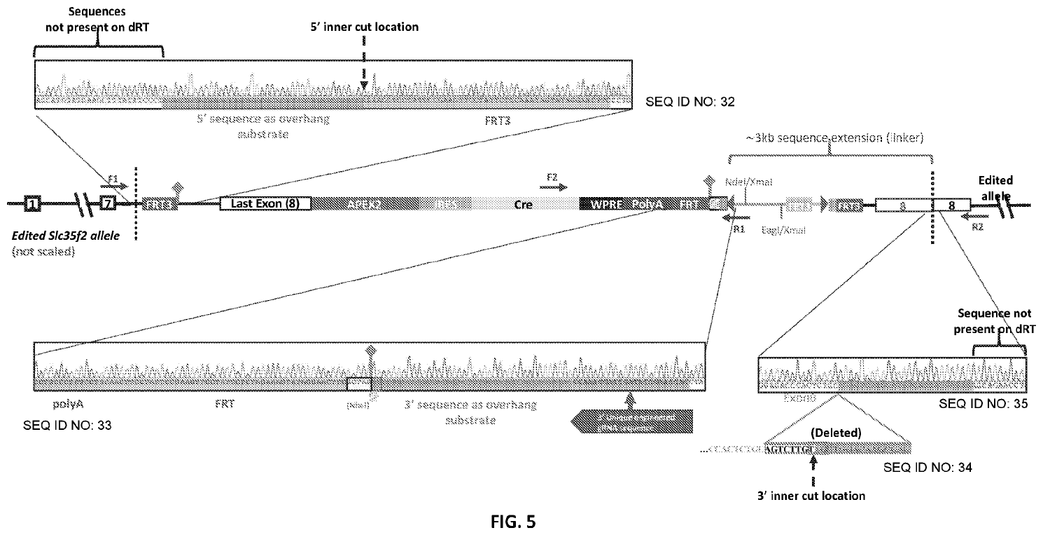

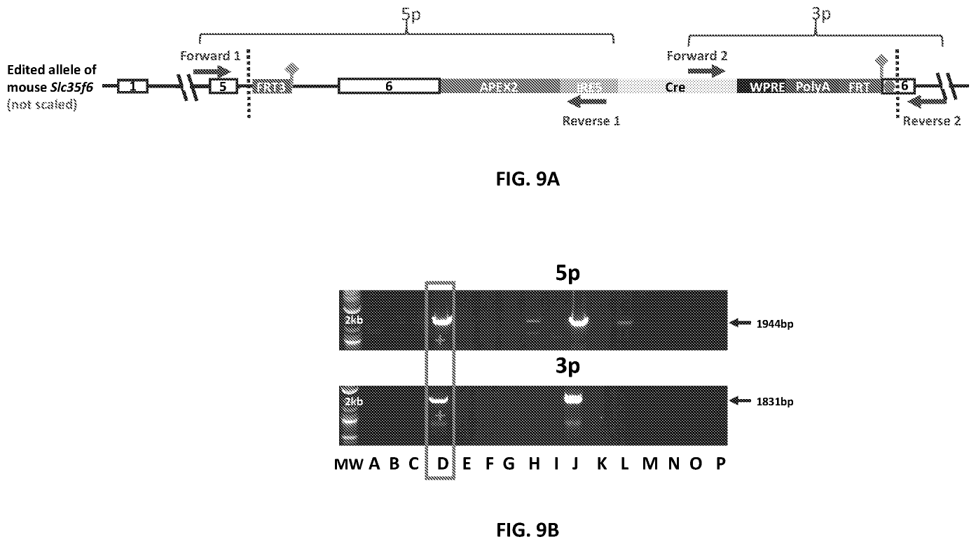

PCR and sequence analysis. The oligonucleotides of primer pairs are either the sequences corresponding to endogenous areas outside the edited fragment in replacement template, or a combination of one primer sequence corresponding to endogenous areas outside the edited fragment with the other primer sequence corresponding to the areas present in the edited fragment, as shown in FIGS. 4A and 9A. Using these primer pairs, the Phusion DNA Polymerase (NEB M0530S, neb dot com) was used for the amplification of genomic DNA fragments according to manufacturer protocols, and amplified PCR products were loaded in agarose gels for size analysis. The bands with predicted size of PCR products were excised, purified (QIAquick Gel Extraction Kit 28704, qiagen dot com) and TA-cloned into a vector with pGEM-Teasy Vector System (Promega A1360, promega dot com) for sequencing (genewiz dot com). Sequencing data were analyzed using Geneious Pro software version 5.0.4 (geneious dot com).

SURVEYOR Assay. The amplified PCR products from animal genomic DNAs were also analyzed with SURVEYOR Mutation Detection Kit (IDT 706020, idtdna dot com) for detections of indels at the endogenous gRNA target sites (either of the inner cut sites). In brief, a 10 μl of PCR products were denatured for 5 min at 100° C., and re-hybridized by slowly cooling to room temperature over the period of one hour, followed by adding 1 of Surveyor assay buffer containing MgCl₂, Surveyor Nuclease & Surveyor Nuclease Enhancer to each samples and incubation for one hour at 42° C. Mismatch mutations were detected when smaller bands were generated after the nuclease treatment, and visualized in an agarose gel. The PCR products containing indels were further analyzed by sequencing in the same way as described above.

### Human Genome Editing

Confirmation of a mutation in exon 20 on the mutant MEDI3L gene allele in MED13L syndrome patient cells. Genomic DNA was extracted from cultured human cell lines of WI-38 normal lung fibroblasts and MED13L Syndrome patient cells of fibroblasts using DNeasy Blood & Tissue Kit (Qiagen #69504) according to manufacturer protocols. Each of the genomic DNA samples was used as templates to produce a DNA fragment by PCR with primers flanking exon 20 of the MED13L gene. The forward primer pJ327 (AGCCTAGTCCAAGTTTTAGAGAG)(SEQ ID NO: 4) and reverse primer pJ328 (AAACTGCCCAGAACACCAAACTGG)(SEQ ID NO: 5) were custom-made (sigmaaldrich dot com). All other primers in the studies were also custom-made using the same source. The PCR primed with pJ327-pJ328 was performed using Fusion High Fidelity DNA Polymerase (NEB #M0530S) according to manufacturer with an Applied Biosystems 2720 Thermal Cycler. A PCR product of 561 bp generated from genomic DNAs of the WI-38 cells and a PCR product of approximate 561 bp produced from genomic DNAs of the MED13L Syndrome patient cells were first incubated with Choice-Taq DNA polymerase (Thomas Scientific #CB4050-1) and then TA cloned into the pGEM-T Easy Vector (Promega #A1360). The cloned PCR products were Sanger Sequenced with M13 Forward (TGTAAAACGACGGCCAGT)(SEQ ID NO: 6) and Reverse (CAGGAAACAGCTATGAC)(SEQ ID NO: 7) primers and the DNA sequence reads from sequencing were analyzed with Geneious software (geneious.com). A single nucleotide addition of thymine in exon 20 of mutant MED13L gene allele in patient cells was revealed, in comparison with the sequence from human WI-38 normal cells, confirming the presence a mutant MED13L allele in the patient cells. The single thymine addition (either by duplicating or inserting) at the codon for Serine results in an animo acid mutation S1497F and consequently reading frame shift which causes an early termination of transcription at exon 21 and production of a truncated MED13L protein with a deletion of approximately 690 amino acids at the C-terminus. The single nucleotide mutation is most likely responsible for the disease of MED13L haploinsufficiency syndrome (MED13L Syndrome).

Identifications of gRNA target sites for iCAP editing to eliminate the single nucleotide mutation. Intronic sequences flanking exon 20 on either the wild type allele or on the mutant allele of MED13L gene are identical and were analyzed to search for potential gRNA target sequences recognized by Cas9 or Cpf1 nucleases using the online tool “Benchling” (benchling dot com). The gRNA target sequences assigned favorable high scores by algorithms for off-target and/or on-target scores were selected as potential gRNA target sites for in vitro assay to validate Cas nuclease's recongniation and cleavage.

In vitro transcription of sgRNAs and validation of gRNA target sites. DNA templates for in vitro transcription of Cas9 sgRNAs were PCR generated with oligoes including a minimal T7 promoter (GCGCGCTAATACGACTCACTATAGG) (SEQ ID NO: 8) and various targets as forward primers (pJ335, pJ336, pJ337 and pJ338) and a gRNA-scaffolding oligo as the reverse primer pJ161 (AAAAGCACCGACTCGGTGCC)(SEQ ID NO: 9) using plasmid pX330-U6-Chimeric_BB-CBh-hSpCas9 (Addgene #42230) as PCR templates. Approximately 120 bp PCR products were produced with various forward primers and the reverse primer and each of the PCR products was used as a template for in vitro transcription of an Cas9 sgRNA with the corresponding gRNA target site in the template. For generations of DNA templates used for in vitro transcription of Cpf1 sgRNAs, a minimal T7 promoter+tracrRNA (nuclease binding scaffold RNA) oligo primer pJ227 (GCGCGCTAATACGACTCACTATAGGTAATTTCTACTAAGTGTAGAT)(SEQ ID NO: 10), was annealed with variously specific crRNA target oligo primers (pJ343, pJ344, pJ345, pJ346, pJ347, pJ348, pJ362, pJ363, and pJ364) containing overlapping sequence with pJ227, followed by extention in a simple PCR reaction to generate approximately 70 bp products. Each of the PCR products was used as a template for in vitro transcription of one Cpf1 sgRNA with the corresponding gRNA target site in the template. PCR products were column purified with either the Qiaquick PCR Purification Kit (Qiagen #28104) for Cas9 templates, or with the QIAEX II Gel Extraction Kit (Qiagen #20021) for Cpf1 templates, and all templates were treated with Proteinase K (0.2 mg/ml with 0.5% SDS) at 50° C. for 30 minutes to remove any traces of contaminating RNAse. In vitro transcription of sgRNAs was performed using the MEGAshortscript T7 Transcription Kit (LifeTechnologies #AM1354) according to manufacturer protocols. The RNA products were column purified with either the MEGAclear Purification Kit (LifeTechnologies #AM1908M) for the Cas9 102 nucleotide RNA products, or the mirVana miRNA isolation kit (Ambion #AM1560) for the Cpf1 44 nucleotide RNA products. The genomic target sequence is a PCR generated 561 bp DNA fragment with primers of J327-pJ328 and genomic DNAs extracted from WI-38 cells and include exon 20 (193 bp), partial 5′ flanking intronic sequence (219 bp) and partial 3′ flanking intronic sequence (149 bp). The intronic sequences were used for gRNA target site search and idenfications. To validate the identified gRNA target sites by in vitro assay, the genomic target sequence, in vitro transcribed sgRNA and assoicated Cas9 (NEB #M0646T) or Cpf1 (NEB #M0653S) nuclease were mixed in 1:10:10 molar ratios respectively, and incubated at 37° C. according to NEB protocols. Results were analyzed on 2% agarose gels with ethidium bromide staining to determine cleavage efficiencies. Validated gRNA target sequences with accurate Cas recongnition and efficient cleavage were chosen and paired to flank exon 20 as the 5′ and 3′ DSB sites according to iCAP editing. The chosen validated gRNA target sites are the guide sequences in oligo primers of pJ335 (GAATCTCCCTTGCTAACCAT) (SEQ ID NO: 11) as 5′ DSB site and pJ338 (ATGTTGCATCTATAAAAGAA) (SEQ ID NO: 12) as 3′ DSB site for Cas9, and in oligoes of pJ343 (GGTTTGATTGCATGTGATAACCC) (SEQ ID NO: 13) as 5′ DSB site and pJ348 (AAAGAAATATAATGTTGCATCTA) (SEQ ID NO: 14) as 3′ DSB site for Cpf1. The validation results from in vitro assay provided molecular bases for constructions of sgRNAs/Cas9 and sgRNAs/Cpf1 expression vectors and for designs and constructions of DNA replacement templates according to iCAP methods.

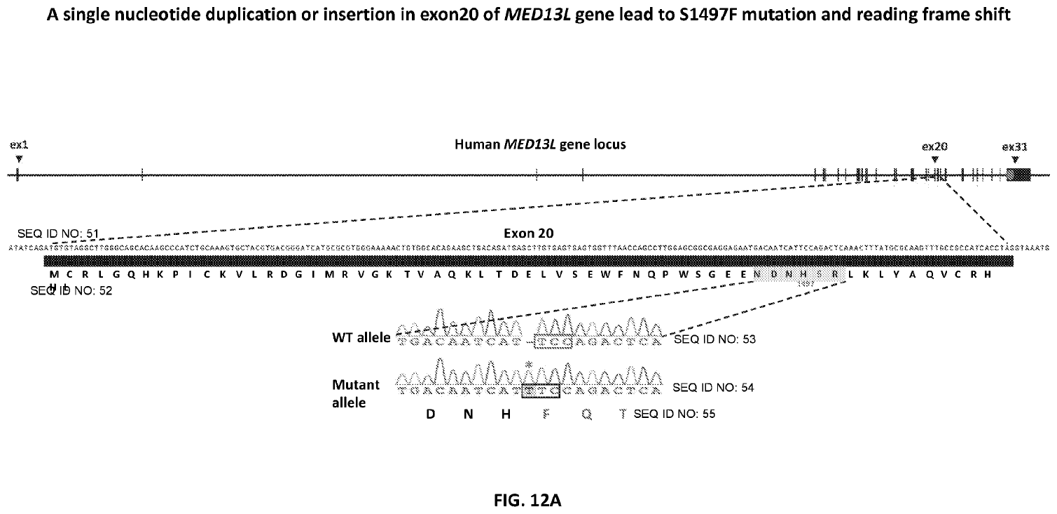

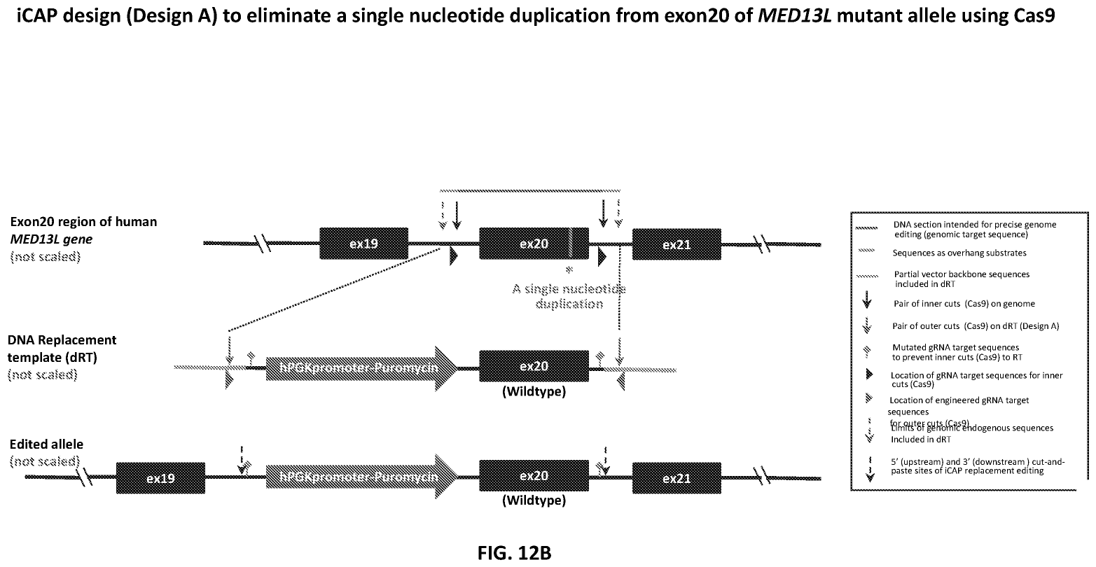

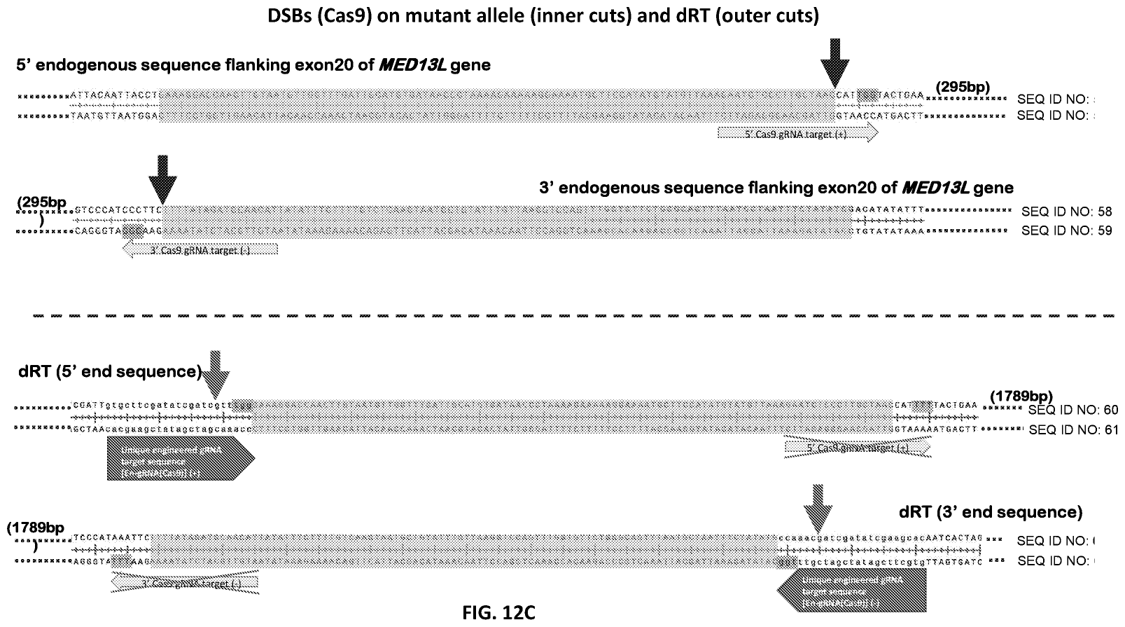

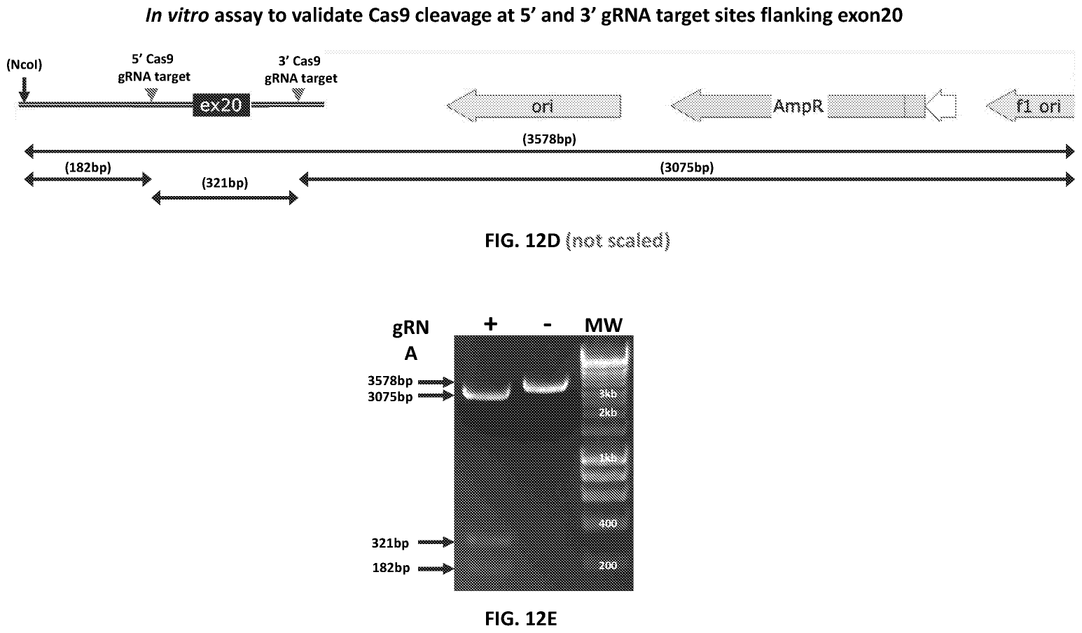

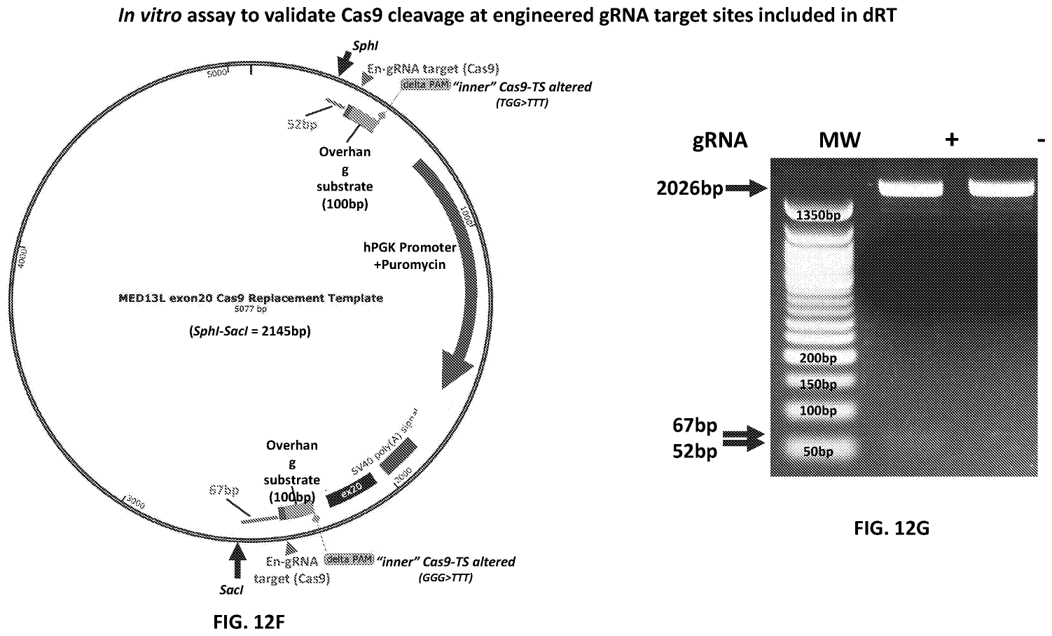

Constructions of sgRNAs Cas9 and sgRNAs Cpf1 expression vectors. In construction of sgRNAs/Cas9 expression vector, two complementary oligoes corresponding to crRNA sequences were denatured in STE buffer (10 mM tris, 1 mM EDTA, 100 mM NaCl) at 100° C. for 10 minutes and then annealed slowly by cooling to room temperature. The annealed oligo products were directionally cloned into the Addgene Multiplex CRISPR/Cas9 Assembly Kit (Addgene #1000000055) as crRNA genes for sgRNA expression. In addition to Cas9 gene, the expression vector as a final plasmid product contains three crRNA genes for expressions of three different sgRNAs with two validated for targeting a 5′ and a 3′ gRNA sites flanking exon 20 of MED13L gene as described above and a third sgRNA validated for targeting an engineered gRNA target site (GTGCTTCGATATCGATCGTT)(SEQ ID NO: 15) placed to flank an edited fragment containing wildtype exon20 and flanking intron sequences within the Cas9 DNA replacement templates (FIG. 12C). To make sgRNAs/Cpf1 expression vector, two complementary oligoes corresponding to crRNA sequences were denatured in STE buffer (10 mM tris, 1 mM EDTA, 100 mM NaCl) at 100° C. for 10 minutes and then annealed slowly by cooling to room temperature. The annealed oligo products were directionally cloned into a modified version of the gRNA+LbCpf1 expression plasmid pTE4398 (Addgene #74042) as crRNA genes for sgRNA expression. In addition to Cpf gene, the vector as a final plasmid product contains two crRNA genes for expressions of two different sgRNAs validated for targeting a 5′ and a 3′ gRNA sites (as described above) flanking exon 20 of MED13L gene and presented on both genome and Cpf1 DNA replacement templates. In both of sgRNAs/Cas9 and sgRNAs/Cpf1 expression vectors, the expressions of crRNA genes are driven by autonomous human U6 promoters and the expressions of nucleases are driven by human CMV promoters. All the inserts cloned into the expression plasmids were confirmed by sequencing.

Construction of DNA replacement templates (dRT). The backbone sequence of dRT is the 561 bp genomic target sequence generated from PCR amplification of wildtype MED13L allele with primers of pJ327-pJ328. The sequence contains wildtype exon 20 of MED13L gene and partial 5′ and 3′ flanking intron sequences as described above and was cloned into a pGEM-T Easy vecotor to generate the plasmid of MED13L ex20 pJ327-328 in pGEM-T Easy. A puromycin resistant gene unit was amplified from plasmid pGL3-U6-sgRNA-PGK-puromycin (Addgene #51133) by PCR and the 1493 bp product was then inserted at an intron EcoRV restriction site just 5′ of MED13L exon 20, resulting in the plasmid of MED13L ex20 pJ327-328+Puro in pGEM-T Easy. SphI-SacI restriction digestions of the plasmid generated the 2139 bp dRT for iCAP editing through usage of Cpf1 (Cpf1 dRT). To construct the Cas9 dRT which contains 100 bp mini-homology sequences as overhang substrates, the MED13L ex20 pJ327-328+Puro in pGEM-T Easy plasmid was used as a template for incorporating modifications necessary for a proper dRT structure, using primer sets of pJ356 (GAAAAAGGAAAATGCTTCCATATGTATGTTAAAGAATCTCCCTTGCTAACCATTT TTACTGAATGAAGGAATGGCTCCTG)(SEQ ID NO: 16) with pJ358 (CTTAACAAATACAGCATTACTTGAGACAAAA GAAATATAATGTTGCATCTATAAAAGAATTTATGGGACGGATTTGCTATTTTAC) (SEQ ID NO: 17) and pJ357 (GTGCTTCGATATCGATCGTTTGGGAAAGGACCAACTTGT AATGTTGGTTTGATTGCATGTGATAACCCTAAAAGAAAAAGGAAAATGCTTCCA) (SEQ ID NO: 18) with pJ359 (GTGCTTCGATATCGATCGTTTGGCATATAGAAATTAGCA TTAAACTGCCCAGAACACCAAACTGGACCTTAACAAATACAGCATTAC)(SEQ ID NO: 19) in sequential PCR reactions. The modifications incorporated in the new dRT resulted in the changes of (i) 100 bp intronic sequences on the 5′ side of the upstream Cas9 gRNA target site and 100 bp intronic sequences on the 3′ side of the downstream Cas9 gRNA target site were tailored to flank the 5′ and 3′ gRNA target sites as overhang substrates (mini-homology arms) by a 58 bp deletion from the 5′ end of upstream intron sequence of exon 20 and a 18 bp addition to the 3′ end of downstream intron sequence of exon 20 as being compared with the original 561 bp genomic target sequence, (ii) the PAM associated with the 5′ and 3′ gRNA target sites were mutated to prevent Cas9 cleavage at the sites (called inner cuts) on dRT and (iii) a uniquely engineered Cas9 gRNA target sites as described above was placed in both ends of 5′ and 3′ mini-homology arms serving as cleavage sites (called outer cuts) for excision a pacthing repair template (an edited fragment) from dRT. The 2060 bp fragment with PCR mediated modifications was TA cloned into the pGEM-T Easy vector to generate a final product of plasmid MED13L ex20 pJ327-328+Puro Cas9 Donor in pGEM-T Easy. SphI-SacI restriction digestions of the plasmid generated the 2145 bp dRT for iCAP editing through usage of Cas9. Structures of both Cpf1 dRT and Cas9 dRT were confirmed by sequencing.

Cell culture and transfection of dRTs and sgRNA Cas expression vectors. Human cell lines of WI-38 lung fibroblasts and MED13L syndrome patient cells of fibroblasts were propagated in culture plates with DMEM (Dulbecco's Modified Eagle Medium, Thermo Fisher Scientific-US) containing 10% fetal bovine serum (Thermo Fisher Scientific-US) and 100 unit/ml penicillin/100 μg/ml streptomycin (Sigma-Aldrich) and maintained in a 37° C. humidified incubator supplied with 5% CO2. For transfection, 1.0×10⁶ of patient cells carrying MED13L mutant allele were suspended in 80 μl of Opti-MEM medium (Invitrogen #31985-062). The cell suspension was combined with 20 μl of genome editing constructs containing 6 μg of either Cpf1 SphI-SacI dRT fragments with 6 μg of the matching sgRNA/Cpf1 expression plasmid or 6 μg of Cas9 SphI-SacI dRT fragments with 6 μg of the matching sgRNA/Cas9 expression plasmid. The combined suspension of 100 μl was electroporated by NEPA21 Electro-Kinetic Transfection System (Bulldog Bio, Portmouth, NH) with parameters of 175 voltage, 2 pulses of 5 msec length and 10% decay rate for pouring pulse and manufacturer pre-set parameters for transfection pulse. The cells transfected with Cpf1 dRT and sgRNA/Cpf1 expression plasmid were labeled as 5-1-2 while the cells transfected with Cas9 dRT and sgRNA/Cas9 expression plasmid were labeled as 4-1-2. After transfection, the cells were cultured in the same culture medium for 48 hours and then selected in the medium with 1 μg/ml of puromycin (Sigma-Aldrich) for 10 days. The surviving cell populations were pooled and harvested for genotypic analysis to examine genome editing at mutant MED13L allele. In another set of experiments, 1.0×10⁶ of patient cells carrying MED13L mutant allele suspended in 80 μl of Opti-MEM medium were combined with either 20 μl of 10 μg sgRNA/Cpf1 expression plasmids or 20 μl of 10 μg sgRNA/Cas9 expression plasmids without dRT included, followed by electroporations in the same parameters. The cell populations transfected with sgRNA/Cpf1 expression plasmids and sgRNA/Cas9 expression plasmids were labeled as iCAP Cpf1 and iCAP Cas9, respectively. These transfected cells were cultured for 24 hours and then harvested for genotypic analysis.

Genotyping & Sequencing. Genomic DNAs were extracted from puromycin selected population pools of 4-1-2 and 5-1-2, respectively, after transfection with editing constructs. Briefly, 50 μl cell samples were lysed in an equal volume of Proteinase K (1 mg/ml) in PBS with incubation on a thermal cycler at 65° C. for 1 hour followed by incubating at 95° C. for 20 minutes. 5 μl of the lysates with genomic DNA extracted was used for PCR genotyping analysis with appropriately designed primers for each unique editing constructs as illustrated in figures. Primer pair of pJ375 (CGATCAGCATACTCACTGCTTCAG (SEQ ID NO: 20), corresponding to 5′ endogenouse genomic sequence not present in the 5′ end of the edited fragment in dRT) and pJ361 (CAGGAGGCCTTCCATCTGTTGCTG (SEQ ID NO: 21), corresponding to sequence of puromycin resistant gene) would yield an approximate 1392 bp fragment, an indication of successful iCAP paste at the 5′ cleavage sites while an approximate 1378 bp fragment generated with the primer pair of pJ360 (AGCTGCAAGAACTCTTCCTCACG (SEQ ID NO: 22), corresponding to sequence of puromycin resistant gene) and pJ355 (GTCTCCTTTCAGACTGATTCCATG (SEQ ID NO: 23), corresponding to 3′ endogenouse genomic sequence not present in the 3′ end of the edited fragment in dRT) suggests successful iCAP paste at the 3′ cleavage sites. Genomic DNAs of transfected cell populations of iCAP Cpf1 and iCAP Cas9 were extracted in the same way, and used for PCR genotypic analysis with the primer pair of pJ375-pJ355 which sequences are corresponding to endogenous genomic DNAs outside the 5′ and 3′ Cpf1 gRNA target sites as well as outside of the 5′ and 3′ Cas9 gRNA target sites in introns flanking exon20 of MED13L gene. A 1080 bp PCR fragment would indicate un-edited allele, whereas deletions would result in a shortened PCR fragment implying cleavages at the upstream and downstream gRNA targets. All PCRs were performed using Fusion High Fidelity DNA Polymerase (NEB #M0530S) according to manufacturer protocols on an Applied Biosystems 2720 Thermal Cycler. PCR fragments with expected size were purified and TA cloned in the pGEM-T Easy Vector (Promega #A136) for sequencing to reveal the details of the edited allele. Sanger Sequencing for cloned PCR fragments were ordered and performed by Genewiz (genewiz.com). All the sequence data obtained were analyzed with Geneious software (geneious.com).

### Example 1: Description of Genomic Editing Using iCAP

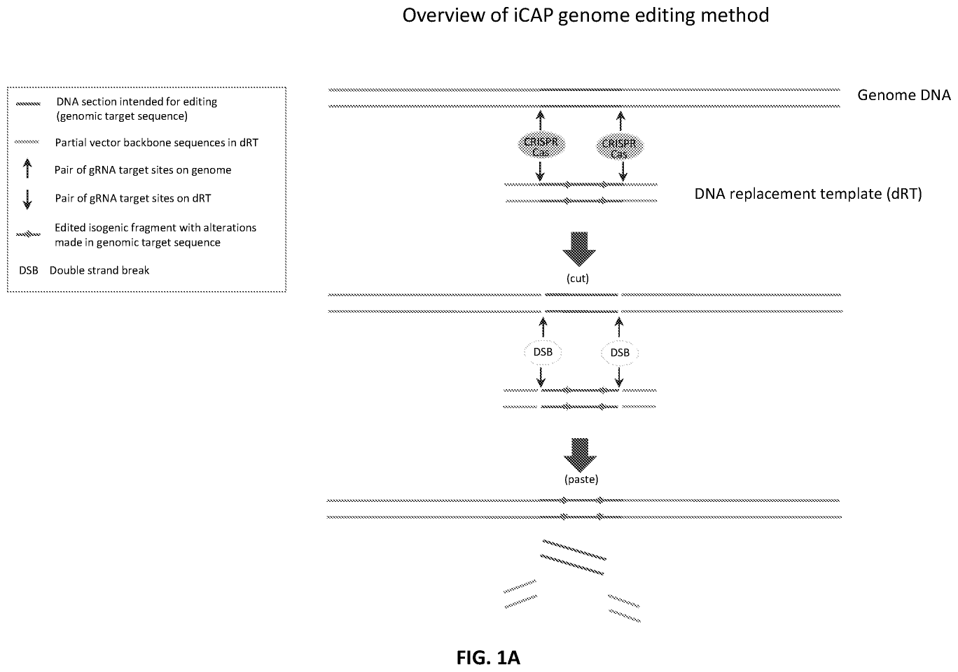

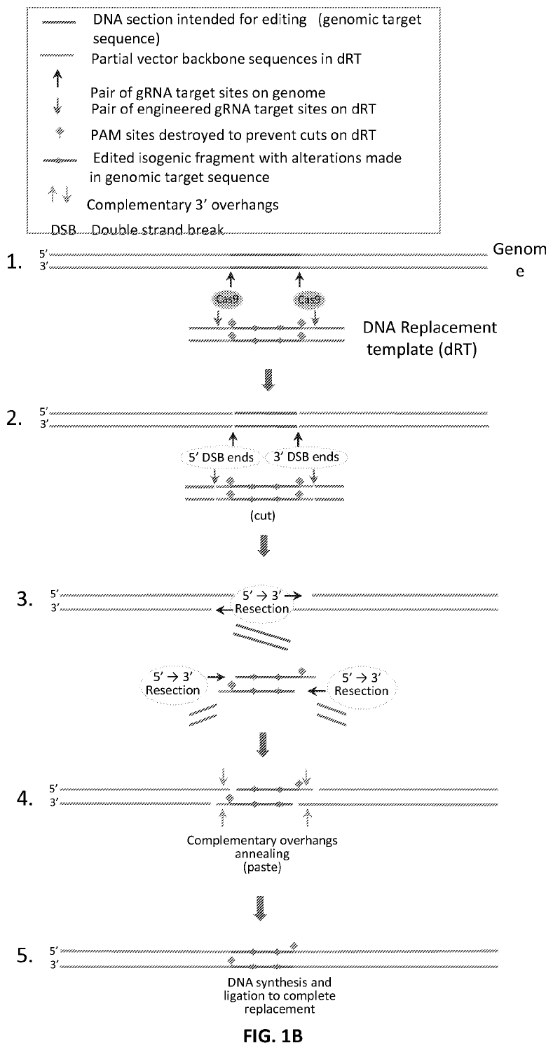

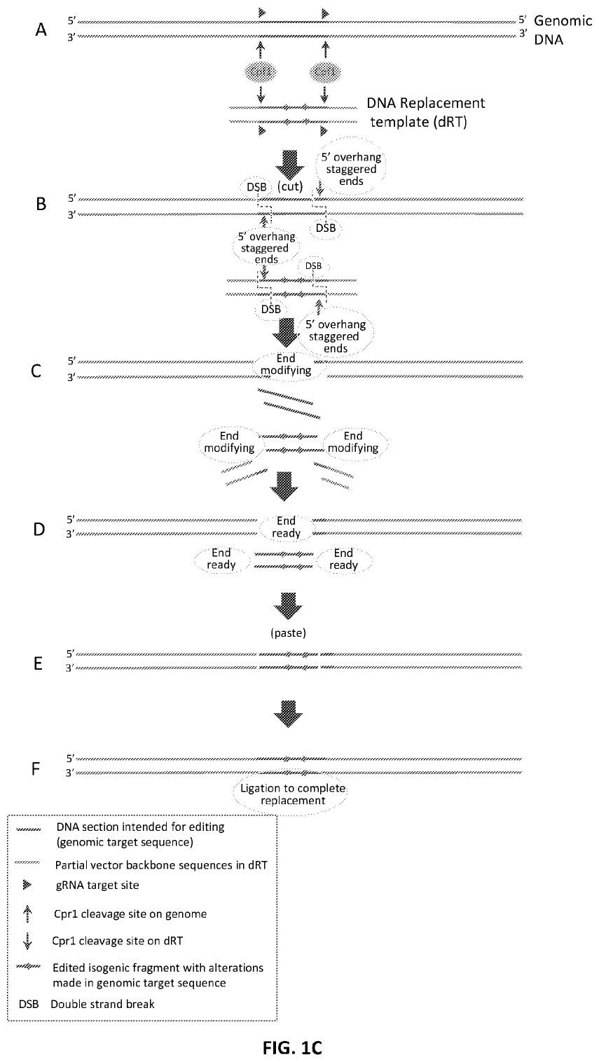

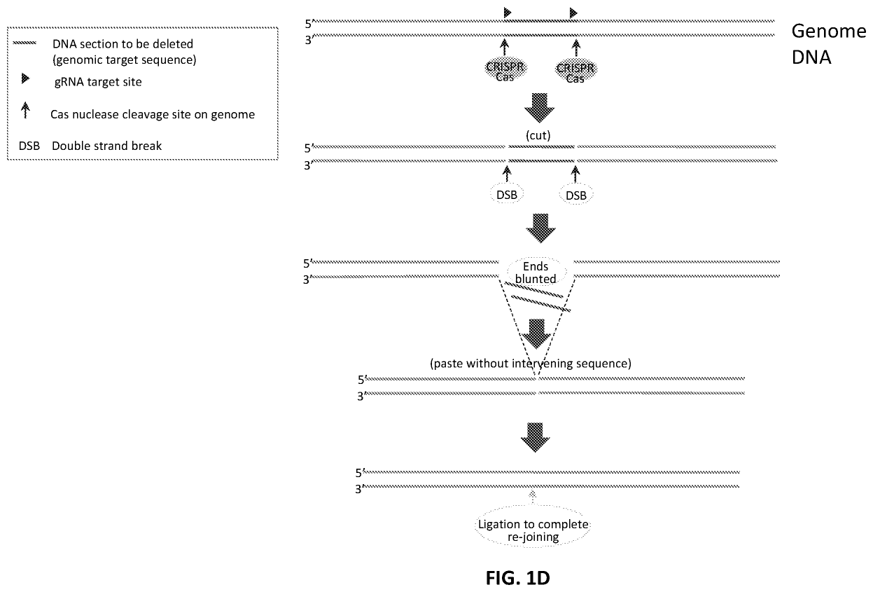

The process of iCAP genomic editing begins by identifying the target endogenous DNA sequence (FIG. 1, step 1). The genomic target sequence is then amplified to produce a section of the endogenous genomic DNA (blue line) spanning the locations for editing and serving as a backbone sequence of a DNA replacement template (dRT). The dRT is then constructed in vitro to edit nucleotide compositions in the backbone sequence which then becomes an edited isogenic fragment with altered nucleotide compositions. Unique gRNA target sites are identified and determined within or on either side of the targeted sequence to act as sites of cleavage by Cas nuclease to excise the endogenous target sequence. The exactly same gRNA target sites are disabled in one design (Design A) of dRT, which contains the backbone sequence with overhang substrate sequences (mini-homology sequences) that are the sequences 5′ adjacent to upstream gRNA target site and 3′ adjacent to downstream gRNA target site on genome, respectively. A specifically engineered gRNA target site is placed in the 5′ side of the 5′ overhang substrate sequences and in the 3′ side of the 3′ overhang substrate sequences to serve as cleavage sites for excision of the edited fragment from dRT (FIG. 1B, step 2). In another design (Design B), the exactly same gRNA target sites on genome are included in the backbone sequence of dRT to act as sites of cleavage by Cas nuclease to excise the backbones sequence with edited nucleotide compositions from dRT (FIG. 1C, step B). The goal of Design A is to create overlapping sequences (serving as overhang substrate) at double strand break (DSB) ends between the endogenous genomic DNA ends and the ends of an edited fragment in dRT when Cas nuclease (Cas9 as illustrated) cleavage occurs at gRNA target sites, and complementary 3′ overhang sequences are generated at the DSB ends when the 5′→3′ end resection occurs in the overlapping sequences and annealing of the complementary 3′ overhang sequences at the DSB ends between genome and the edited fragment results in a repaired genome with the intervening sequence replaced by the backbone sequence with altered nucleotide compositions. As the backbone sequence can be easier reconstructed in-vitro to include edited nucleotide compositions, there is no limitation in types of nucleotide changes and sequence alterations, and the changes and/or alterations (Illustrated as asterisks in FIG. 1) can be made at more than one location along the DNA section. Next, Cas9 nuclease induces two double strand breaks (DSBs) on both the genome (inner cuts shown as black arrows in Design A) and the dRT (outer cuts shown as blue arrows in Design A) at the gRNA target sites. Blunt ends are formed at DSB sites when Cas9 induces cleavage or 5′ overhang ends are created at DSB sites when Cas12a (Cpf1) induces cleavage. Next, in Design A, 5′→3′ end resection take places at the blunt ends of the edited DNA fragment excised from the dRT just as it occurs to blunt ends of DSB sites on the genome. This step occurs as part of the normal DNA repair machinery and results in the formation of complementary 3′ overhangs (indicated by orange arrows) at these ends. Then, the 3′ overhangs of the edited DNA fragment anneal to the corresponding ends present at both of the upstream (5′) and downstream (3′) DSB ends of genome to re-join the broken genome, therefore replacing the lost section of endogenous DNA. DNA synthesis and ligation at the annealing sites are followed to complete the replacement, resulting in the edited nucleotide composition incorporated into the genome. While in Design B, blunt ends (Cas9 cleavage) of genome and the edited fragment are linked at 5′ and 3′ cutting sites, respectively, followed by ligations to complete end joining; whereas the 5′ overhang ends are created at DSB induced by Cpf1 (FIG. 1C) and the 5′ overhang ends, regardless of compatible or non-compatible, may need to be processed to become blunt ended to facilitate the linkage of the edited fragment with the broken genome (FIG. 1C, Step C-D). Once the linkage is made, end ligations are followed to complete end-rejoining.

### Example 2: DNA Replacement Template (Donor) Design A for Insertions of a FRT3 and an APEX2-IRES-CRE Expression Cassette at Two Locations in Slc35f2 Gene by iCAP

As an example of the use of the iCAP genomic editing to precisely insert a large DNA construct with alterations at two locations into the mouse genome in situ, a study was then conducted in which a 48 bp FRT3 site and a 3.7 kb APEX2-IRES-CRE expression cassette ending with a FRT site were inserted into the mouse slc35f2 locus at two locations, respectively. The construct was synthesized to form the edited fragment residing in the DNA replacement template (dRT) as illustrated in FIG. 2A. gRNAs and Cas9 nuclease created four DSBs, two within the endogenous slc35f2 locus & two flanking the edited fragment on the dRT. FIG. 2B, the edited fragment includes 40 bp sequences (orange shaded) as overhang substrates (homology arms), both 5′ and 3′ ends, matching sequences immediately upstream & downstream of the respective genomic DSB locations (vertical arrows). FIG. 2A, bottom also shows an illustration of the successfully edited endogenous slc35f2 locus. Shown in FIG. 4A is the position of primers (purple arrows) designed for identification of the new allele by PCR, and located both outside the range of the overhang substrates (homology arms) and within the new sequence of inserted expression cassette. An in vitro study was then conducted to verify the function of the gRNAs and Cas9 nuclease. For the genomic target sequence, PCR was used to amplify a DNA fragment spanning the target sequence (FIG. 2C), which was then cleaved with gRNA and Cas9 in vitro. Separation of the resulting reaction via agarose gel revealed DNA fragments of the expected sizes (FIG. 2D). A similar in vitro gRNA/Cas9 reaction was performed using the vector containing the dRT (FIG. 2E), which also generated DNA fragments of the expected sizes (FIG. 2F). Together, these data indicated the successful design of the gRNAs and the dRT necessary for the iCAP genome editing.

### Example 3: iCAP Editing of the Mouse slc35f2 Locus

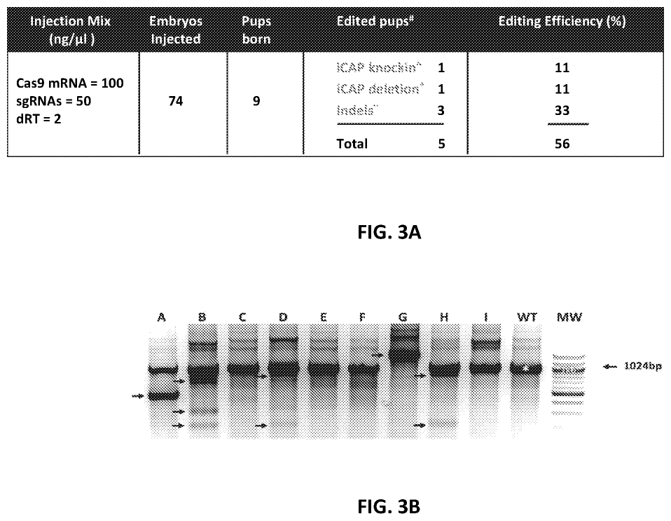

A total of 74 one-cell stage embryos of strain B6D2F1 were injected with a buffer mixture (Cas9 mRNA, 3 sgRNAs, and DNA replacement template excised from a plasmid with restriction enzymes NdeI and XmaI). Surviving embryos were re-implanted into the oviduct of pseudo pregnant surrogate mothers for development to term. A total of 9 pups were born. Genotyping by PCR & DNA sequencing was performed on biopsy samples collected at 3 weeks of age. A SURVEYOR Nuclease assay testing for evidence of CRISPR/Cas9 mediated DSBs revealed 5/9 animals (FIG. 3B, Animals A,B,D,G&H; 56%) showed signs of Cas9 mediated genome editing (arrows). PCR of the wildtype allele, unaffected by SURVEYOR Nuclease, is indicated in lane WT. A table summarizing the outcome of the microinjection and animal production studies is shown in FIG. 3A.

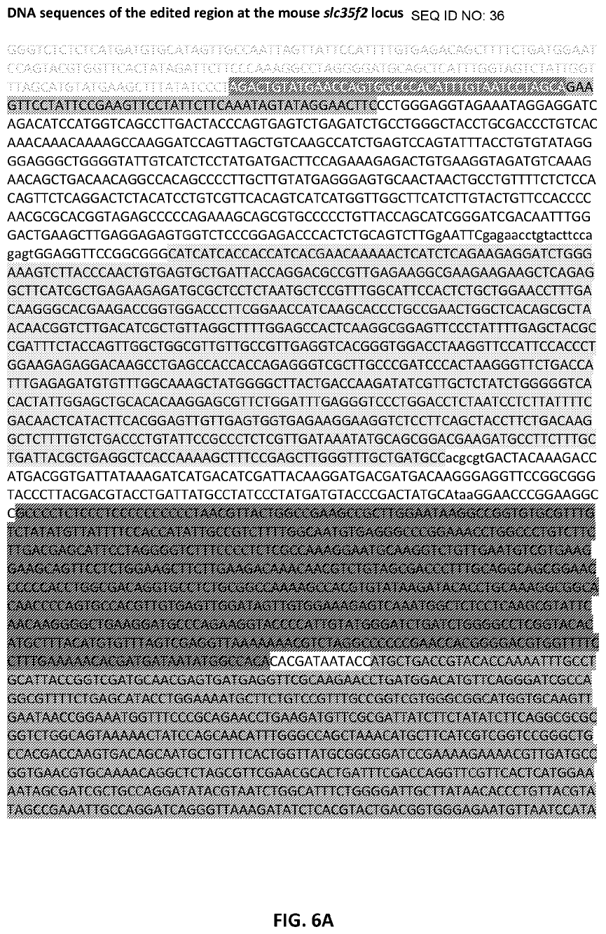

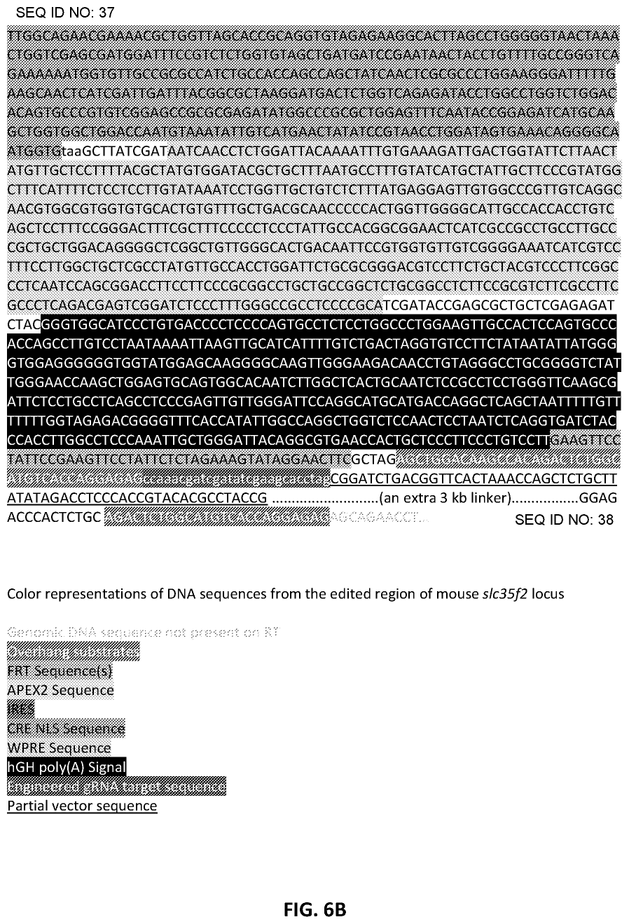

The presence of edited slc35f2 alleles in the nine surviving animals was then verified by PCR. FIG. 4A. shows the predicted structure of the successfully edited slc35f2 allele. Primers for genotyping the 5′ and 3′ ends of the inserted gene are indicated, along with the size of any resulting PCR products (5′=1768 bp, 3′=1850 bp). Vertical dashed lines indicate the limits of homology present in the donor fragment. FIG. 4B shows results of genotyping PCRs done on the 9 pups (A-I) for identification of successful insertion at the 5′ and 3′ DSBs. 1/9 pups (Animal G; 11%) was positive for both PCR reactions, although the PCR product for the 3′ end of the insertion was considerably larger than expected. Animal G was then subjected to further DNA sequencing of the 5′ and 3′ ends of the inserted construct (FIG. 5) As shown in the upper sequence panel, the paste at 5′ DSB ends between genome and the edited DNA fragment is seamless, as the 5′ DNA sequence not present in the dRT was flawlessly rejoined with a full overhang substrate sequence followed by the FRT3 site and intron sequences as exactly constructed in the dRT. The lower sequence panel shows the paste at 3′ DSB ends between genome and the edited DNA fragment. As shown, the DSB occurred at the gRNA target site on genomic DNA resulting in a 15 bp deletion of overhang substrate sequence toward 3′ direction at this inner cut site, but not at the engineered gRNA target site on the dRT as this gRNA target sequence were intact, fully retained and flanked by the 3′ overhang substrate and partial vector backbone sequences just as pre-constructed resulting in an unavailability of the 3′ overhang-substrate-flanking DBS end. The dRT's uncut 3′ end, which contains the 23 bp uncut 3′ engineered gRNA target site and a 359 bp partial vector backbone sequence with NdeI restriction site at the end, was transited downstream to an additional ˜2.7 kb sequence. About two thirds (˜1.7 kb) of the additional sequence in the 5′ and middle portions is a section of vector backbone sequence that was not included in the dRT but in the plasmid bearing the dRT. It is likely that the prepared dRT for microinjection was contaminated with either the plasmid or a XmaI-EagI vector backbone fragment (FIG. 2E) as the ˜1.7 kb DNA in the additional sequence is identical to the XmaI-EagI fragment with ˜240 bp lost at EagI end. This fragment was also produced during dRT preparation from the plasmid as the EagI was used to break the vector backbone in half to facilitate dRT isolation. The 3′ end of the ˜1.7 kb DNA was transited downstream to the rest (˜1.0 kb) of the additional sequence starting with the dRT's uncut 5′ end of another copy of the construct through the disrupted 3′ gRNA target site ending prior to the expression cassette (XmaI-exon8 fragment, FIG. 2E). The ˜1.0 kb XmaI-exon8 fragment contains two FRT3 sites (one originally in the 5′ partial vector backbone sequence included in dRT between XmaI and 5′ engineered gRNA target site, and the other in the intron between 5′ overhang substrate sequence and exon8), intron and coding sequence of exon8. It is the 3′ end of the ˜2.7 kb additional DNA that was re-joined with the genome at the 3′ paste site. Although the edited isogenic fragment pasted was extended at the 3′ end with the total of ˜3 kb sequence (FIG. 5, middle), the edited allele remains to be a result of iCAP replacement and the structure of intended alterations at the edited locus remains unchanged and as designed for (i) inserting a FRT3 site in the intron upstream of the last exon and (ii) in-frame inserting an expression cassette with built-in stop codons and poly(A) signal sequences into the immediate 5′ side of the endogenous stop codon, which led to disruptions of the 3′ non-coding sequence of the last exon anyway. The addition of the extra ˜3 kb DNA contributed a much larger PCR product (+* marked band in FIG. 4B) as seen in genotyping using the primers for the 3′ paste site. The sequence data of the flanking genomic DNA and the edited fragment in the edited allele are illustrated in FIGS. 6A-6B. In total, these data illustrate the successful use of the iCAP process to quickly and precisely introduce a large DNA construct into a targeted locus in mouse embryos, efficiently generating transgenic animals.

### Example 4: Strategy (Design A) for Editing the Mouse slc35f6 Locus by iCAP

As a further example of the use of iCAP to efficiently edit genomic DNA, a follow-up study was designed in which the slc35f6 locus was targeted for editing. FIG. 7A depicts a map of the slc35f6 locus, the layout of the DNA replacement template, and the resulting edited locus. The alterations of the intended and precise genome editing are (1) to place a conditional 48 bp FRT3 site in the intron area between the last two exons (exon5 and 6) and (2) in-frame to insert an expression cassette of a 3.7 kb APEX2-3×Flag-2XHA-IRES-Cre-WPRE-polyA-FRT fragment into the immediate 3′ side of the codon for the last amino acid of slc35f6 gene. Black triangles, gRNA target sequences for inner cuts on genome; Blue triangles, gRNA target sequences for outer cuts on dRT.

Similarly, FIG. 7B depicts the endogenous slc35f6 genomic DNA sequences where gRNA target sites designed for inner cuts (black arrows) by Cas9 are located and the DNA replacement template sequences where engineered gRNA target sites designed for outer cuts (blue arrows) by Cas9 are located. Sequences shadowed with orange color are overhang substrates (homologous sequences) presented on both of the endogenous gene and the edited fragment residing in RT, and 3′ overhang sequences are derived from the substrates after the 5′→3′ end resection initiated by DNA cleavages inside mouse zygotes. The genomic gRNA target sites (sequences) for inner cuts on dRT are interrupted and disabled by insertions of a FRT3 in the upstream gRNA target site and an expression cassette in the downstream gRNA target site allowing only outer cuts to be induced. The engineered gRNA target sites (sequences) for outer cuts are designed either with completely engineered sequences or with hybrid sequences (partial endogenously existing sequences and partial engineered) for the outer cuts to occur only on dRT but not on genome. With such a uniquely specific and coordinated design of the cleavage sites, the overhang substrates are subject to the 5′→3′ resection after both pairs of inner and outer cuts are induced. As shown, the endogenous section of 579 bp DNA is excised from the slc35f6 locus by inner cuts and the edited DNA fragment of 4.5 kb is released from RT by outer cuts, upon introductions of Crispr/Cas9 components and dRT into mouse zygotes.

In FIG. 7C, the annotated 3′ genomic region for slc35f6 is shown, highlighting the position of the final two exons (white boxes), position of the endogenous STOP codon (red octagon), validated “inner” gRNA target sites #1 & #2 (black arrows), and position of primers (For/Rev) for creation of a Cas9 assay PCR fragment & genotyping (purple arrows). gRNA #1 creates a DSB within the final intron of slc35f6 136 bp upstream of the final exon, and gRNA #2 creates a DSB 27 bp upstream of the endogenous STOP codon. An agarose gel (FIG. 7D) shows the results of an in vitro Cas9 assay. The 1105 bp PCR fragment was generated using the primers shown above is cleaved by the active gRNA/Cas9 enzyme complexes in Lane 1, resulting in 3 bands (579 bp, 305 bp & 221 bp; black arrows) as expected. Lane 3 shows the uncleaved PCR fragment. (Lane 1—PCR fragment+(gRNA #1+gRNA #2+Cas9); Lane 2—DNA size marker; Lane 3—Uncut PCR fragment).

FIG. 7E illustrates the 9581 bp plasmid with pre-constructed DNA replacement template for slc35f6 showing the position of the engineered gRNA target sites (blue arrows) for outer cuts, overhang substrate sequences (orange boxes), flanking FRT sites (blue arrowed boxes), final exon (white arrowed box), in-frame expression cassette (APEX2 tag, 3×FLAG, 2HA, IRES, Cre NLS, WPRE, hGH poly(A) signal), and relocated endogenous STOP codon (red octagon). When successfully cut at the “outer cut” target sites (blue arrows), a 4501 bp fragment is created as the repair (edited) fragment which entire sequence has been confirmed by sequencing. The results of the in vitro Cas9 assay are shown in FIG. 7F. The 9581 bp plasmid is cleaved by the active outer gRNA/Cas9 enzyme complexes in Lanes #1 & #3 resulting in two smaller fragments (5080 bp+4501 bp; black arrows) as expected. Including the endogenous gRNAs #1 & #2 in the assay (lanes #2) does not result in any DSB (lanes #2) or extra DSB formation (lanes #3), indicating that those sites have been successfully altered in the repair fragment and should not be cleaved in vivo. (Lane 1—plasmid DNA+(outer gRNA+Cas9); Lane 2—plasmid DNA+(gRNA #1+gRNA #2+Cas9); Lane 3—plasmid DNA+(outer gRNA+gRNA #1+gRNA #2+Cas9); Lane 4—Uncut plasmid; Lane 5—DNA size marker).

### Example 5: Successful Editing of the slc35f6 Locus by iCAP

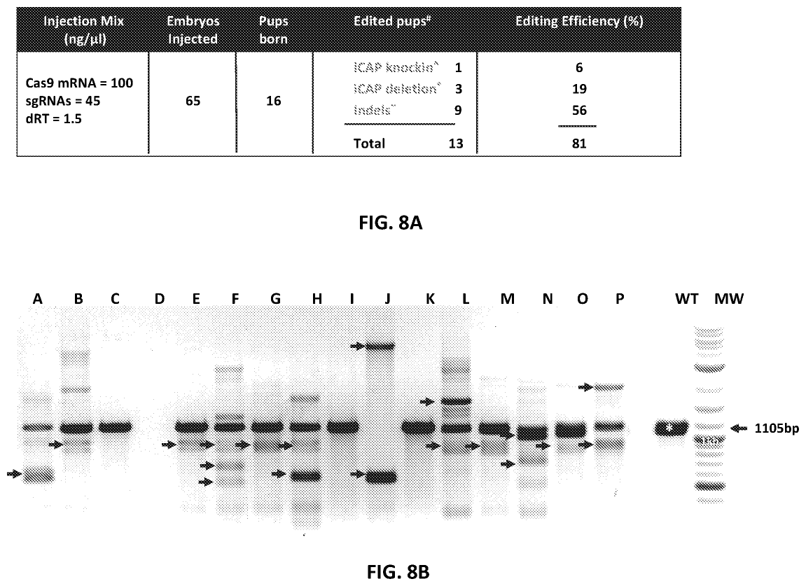

A total of 65 one-cell stage embryos of strain B6D2F1 were injected with a buffer mixture (Cas9 mRNA, 3 sgRNAs, and DNA replacement template excised from a plasmid with restriction enzymes NdeI and XmaI). Surviving embryos were re-implanted into the oviduct of pseudo pregnant surrogate mothers for development to term. A total of 16 pups were born. Genotyping by PCR & DNA sequencing was performed on biopsy samples collected at 3 weeks of age. A table summarizing the outcome of the microinjection and animal production studies is shown in FIG. 8A.

In order to screen the resulting pups, a SURVEYOR Nuclease assay was performed to verify evidence of CRISPR/Cas9 mediated DSBs occurred on the targeted genomic DNA region amplified using the same PCR primers as shown in FIG. 7C. The assay results in FIG. 8B revealed 13/16 animals (Animals A,B,D,E,F,G,H,J,L,M,N,O&P; 81%) showed signs of Cas9 mediated genome editing (arrows). PCR of the wildtype allele, unaffected by SURVEYOR Nuclease, is indicated in lane WT. Sequencing of the PCR products confirmed the assay results. To identify animals with iCAP edited slc35f6 allele which contains a large inserted exogenous expression cassette and might be unfavorable to SURVEYOR assay, A PCR genotypic screen using different sets of primers was performed, and the primer sets and the genotyping results are shown in FIG. 9. Although animal D did not produce any band in the SURVEYOR assay, the PCR screening for the knock-in of the repair fragment was positive for the animal.

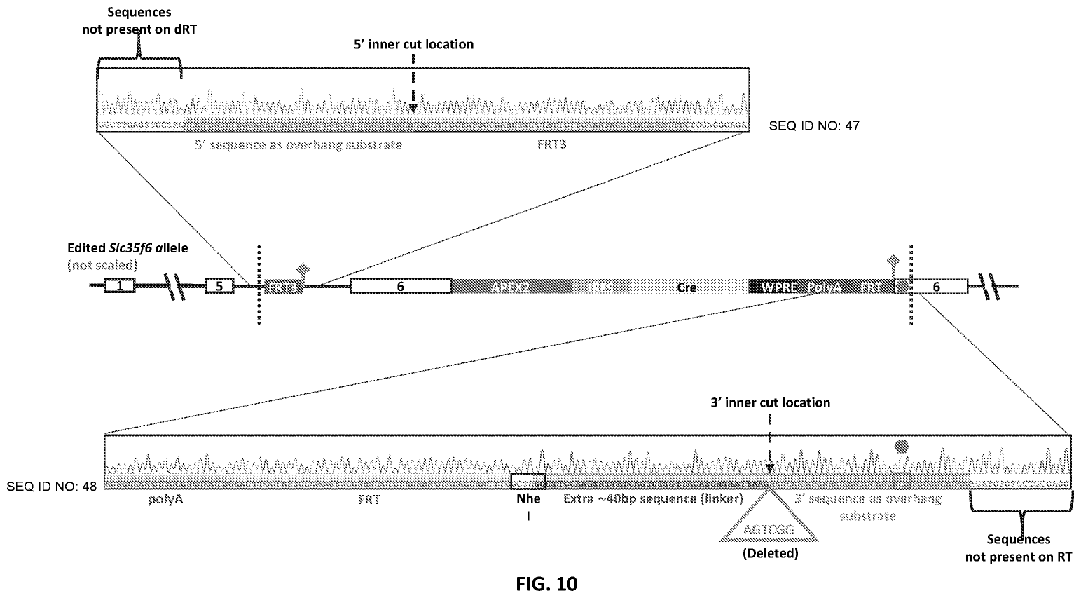

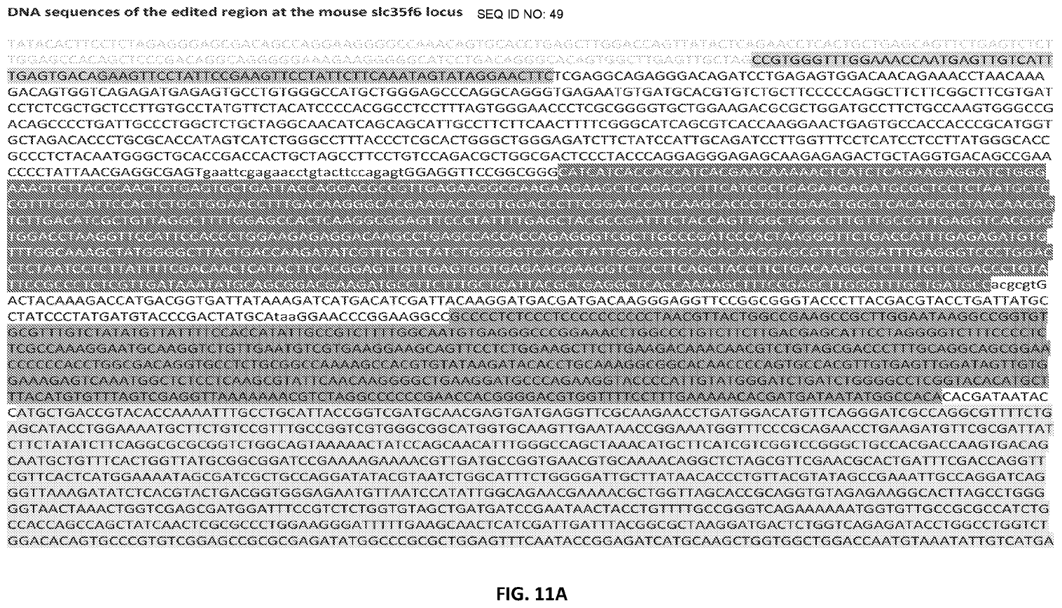

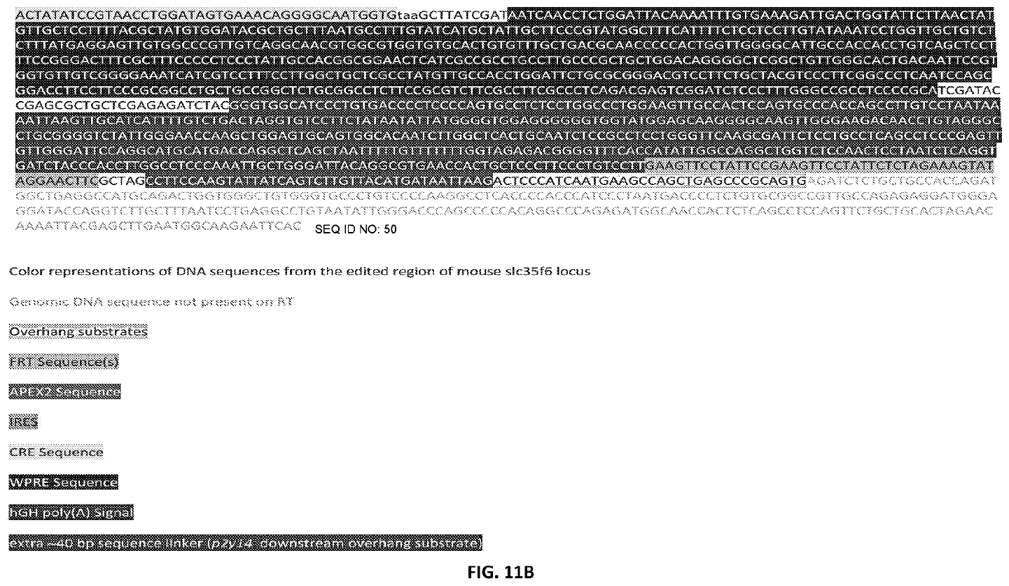

FIG. 9A shows the predicted structure of the successfully edited slc35f6 allele. Primers for genotyping the 5′ and 3′ ends of the inserted gene fragment are indicated (purple arrows). The expected size of positive PCR products are 1944 bp (For/Rev 1) and 1831 bp (For/Rev 2). Vertical dashed lines indicate the limits of homology present in the replacement template. Results of genotyping PCRs done on the 16 pups (A-P) revealed a successful iCAP replacement of genomic intervening sequence with the repair (edited) fragment in Animal D (FIG. 9B). Follow-up sequencing confirmed that the repair (edited) fragment excised from the DNA replacement template had successfully replaced the endogenous homolog section of the slc35f6 gene via iCAP replacement. Genomic DNA sequencing of the slc35f6 allele was conducted for Animal D (FIG. 10). As shown in the upper sequence panel, the paste at 5′ DSB ends between genome and the edited DNA fragment is seamless as the 5′ DNA sequence not present in dRT was flawlessly transited downstream to the 5′ overhang substrate sequence in full length followed by a FRT3 site and intron sequences as exactly constructed in dRT. The lower sequence panel shows the paste at 3′ DSB ends between genome and the edited DNA fragment. As shown, the 3′ DNA sequence not present in dRT was flawlessly transited upstream to the 3′ overhang substrate sequence which lost 6 bp nucleotides at the 3′ inner cut site (3′ DBS) as indicated in an orange triangle box. Continuously upstream from the truncated end of the 3′ overhang substrate sequence is a small section of ˜40 bp nucleotide sequence transited upstream to NheI restriction site, FRT and poly(A) that are the sequences at the 3′ end of the edited fragment. The results indicate that the excisions of the genomic target sequence and the edited isogenic fragment occurred within the mouse embryo and the edited slc35f6 allele in Animal D was a result of iCAP replacement. The ˜40 bp nucleotide sequence is not a random sequence as it is identical to the downstream overhang substrate sequence of another gene dRT (p2y14) that was also built at the same time as the slc35f6 and co-injected into zygotes. It might serve as a linker to bridge the 3′ DSB ends on genome and on the edited fragment in a scenario in which the 3′ overhang substrate sequence at the 3′ end of the edited DNA fragment might be deleted completely during repair steps at its damaged end. Although the ˜40 bp nucleotide sequence was added at the 3′ paste site, the structure of intended alterations at the edited locus however remains unchanged and the functionality of the edited locus is not compromised either as further assay determined that the transcripts corresponding to edited coding area of last exon and in-frame expression cassette were produced from the edited locus in the animal. In addition, the addition of the extra small linker indirectly indicates that the intended and precise editing at the two locations of slc35f6 locus was a result of iCAP rather than other pathways such as a homologous recombination. The full sequence of the iCAP edited region and flanking genomic sequences is shown in FIG. 11A-11B with the various features highlighted.

### Example 6: iCAP DNA Replacement Template (Donor) Design A for Deletion of a Single Disease-Causing Nucleotide Duplication from Exon20 of Human Mutant MED13L Allele in Patient Cells

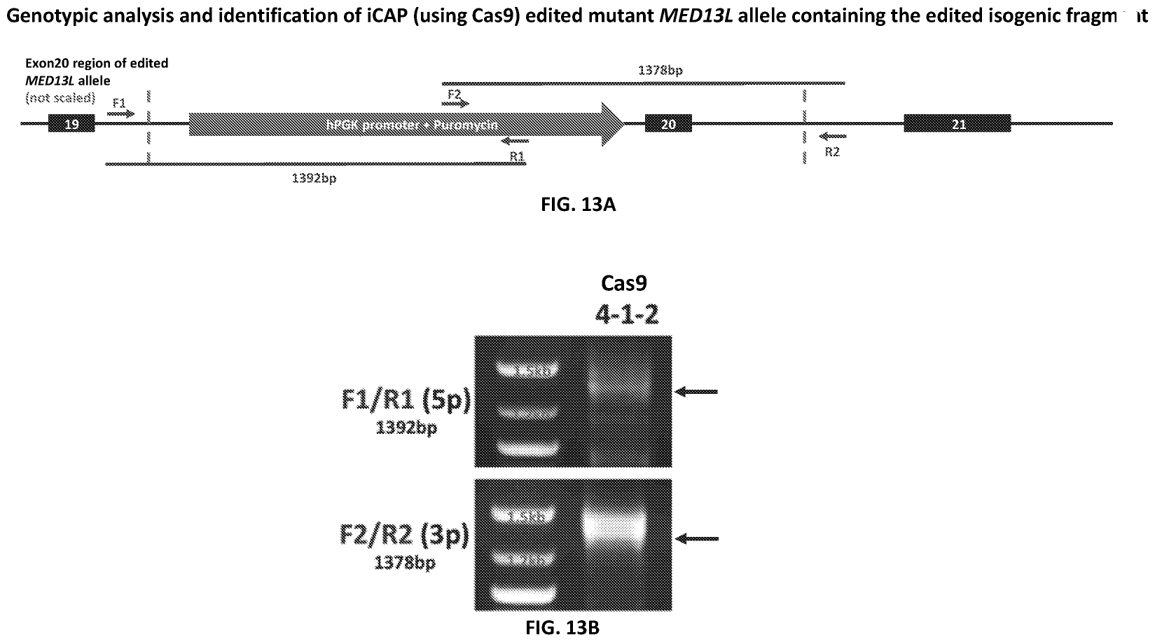

As an example of the use of the iCAP genomic editing to precisely alter genome sequence at the level of individual nucleotides in human genome, a study was conducted to eliminate a single nucleotide duplication from exon20 of MED13L gene in genome of patient cells. The single base Thymine duplication in coding sequence (FIG. 12A) causes reading frame shift at Serine (S1497F) and consequently early termination of transcription, resulting in productions of truncated MED13L protein products with clinic manifestation of MED13L Syndrome. In this study, a dRT was constructed to include (1) the edited fragment which was synthesized with the components, in the 5′ to 3′ direction, an engineered gRNA target site+partial 5′ flanking intron sequence containing a selection marker puromycin resistant gene+wildtype exon20 (without the single Thymine duplication)+partial 3′ flanking intron sequence+an engineered gRNA target site and (2) partial vector backbone sequences of 52 bp and 67 bp flanking 5′ and 3′ ends of the edited fragment (FIG. 12B, middle, and FIG. 12F). The iCAP design allows the mutated exon20 of MED13L gene to be replaced by a wild-type exon20, resulting in a deletion of the single disease-causing nucleotide duplication in the edited allele as illustrated in FIG. 12B, bottom). The intron sequences of 100 bp (shaded in FIG. 12C, bottom) present on both 5′ and 3′ ends of the edited fragment match to the endogenous sequences immediately upstream & downstream of the respective genomic DSB sites (vertical black arrows in FIG. 12C, top), and serve as overhang substrates (mini-homology arms) for creating 3′ overhang annealing strand upon 5′→3′ end resection at DSB ends. In iCAP process, Cas9 induces two DSBs (vertical black arrows in FIG. 12C, top) on endogenous genome sequence at a 5′ and a 3′ gRNA target sites (inner cuts), and two DSBs (vertical arrows in FIG. 12C, bottom) on dRT at engineered gRNA target sites (outer cuts) other than at the genomic gRNA target sites which were intentionally disabled by mutating PAM sites (shaded in green) when pre-constructing the dRT. Shown in FIG. 13A is the position of primers (arrows) designed for identification of the newly edited allele by PCR, and located both outside the range of the overhang substrates and within the new sequence of puromycin resistance gene. An in vitro study was then conducted to verify the function of the gRNAs and Cas9 nuclease. For production of the genomic target sequence, PCR was used to amplify a DNA fragment spanning the wildtype exon 20 of MED13L gene, and the PCR product was then cloned in a vector and cleaved with sgRNAs and Cas9 in vitro (FIG. 12D). Separation of the resulting reaction via agarose gel revealed DNA fragments of the expected sizes (FIG. 12E). A similar in vitro sgRNA/Cas9 reaction was performed using the vector containing the dRT (FIG. 12F), and the assay also generated DNA fragments of the expected sizes (FIG. 12G). Together, these data indicated the successful design of the gRNAs and the dRT necessary for the iCAP genome editing of the mutant MED13L allele in patient cells.

### Example 7: Successful iCAP Editing to Eliminate a Single Disease-Causing Nucleotide Duplication from Exon20 of Human Mutant MED13L Allele in Patient Cells

Patient cells of fibroblasts carrying mutant MED13L gene allele were transfected with a sgRNAs/Cas9 expression vector and the dRT, which is a SphI-SacI fragment (FIG. 12F) and A Design (FIG. 12B), by electroporation, followed by incubation in culture media for 48 hours and then in selection medium with puromycin. The surviving cell populations (labeled as 4-1-2) were harvested and genomic DNAs were extracted for genotypic analysis.

Using primers F1-R1 and F2-R2 as shown in FIG. 13A, PCR products of a 1392 bp and 1378 bp were generated as indicated by horizontal black arrows in FIG. 13B. The PCR fragments span the 5′ (upstream) and 3′ (downstream) paste (re-joining) sites to puromycin resistant gene, respectively, indicating the presence of the edited allele as a result of a replacement of the endogenous mutant exon20 with the edited fragment containing a wild-type exon20 and the puromycin resistant gene. Vertical dashed lines in FIG. 13A indicate the limits of overhang substrates (mini-homology arms) present in the edited fragment excised from dRT, and horizontal purple arrows represent forward (F) and reverse (R) primers.

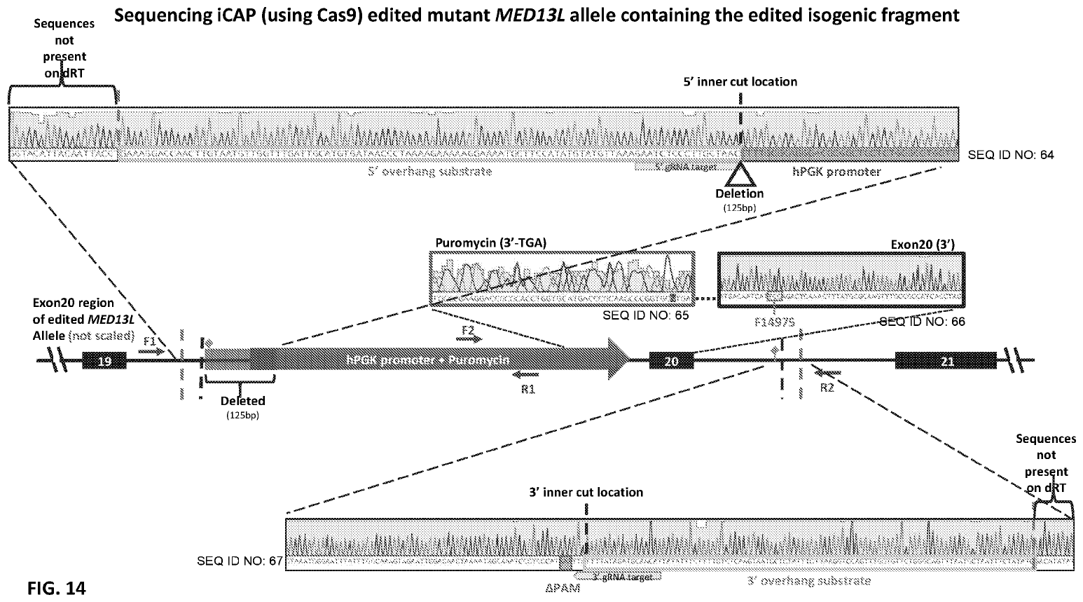

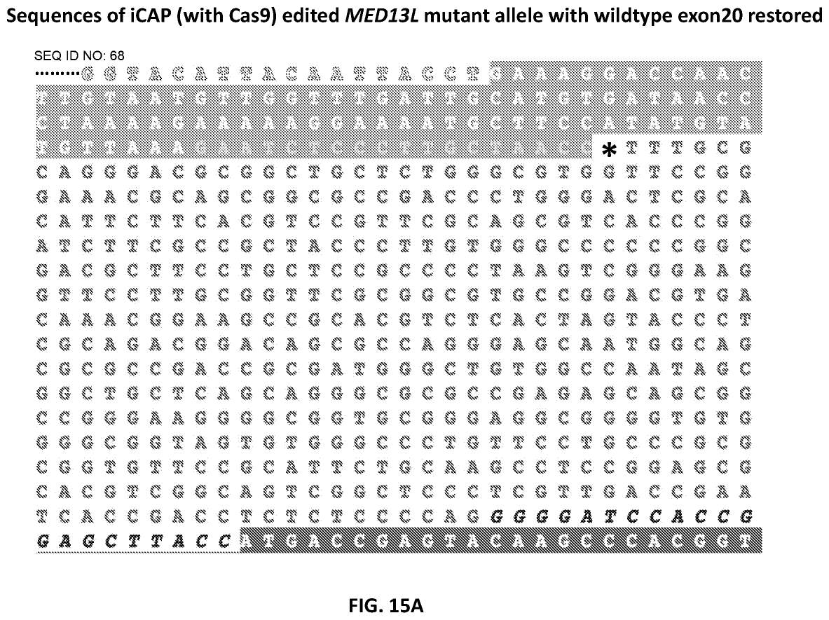

The predicted structure of the successfully edited MED13L allele by PCR genotyping was further analyzed by DNA sequencing. As shown in the bottom sequence panel of FIG. 14, the paste at 3′ DSB ends (vertical black dash line) between genome and the edited DNA fragment is seamless, as the 3′ genomic DNA sequence not present in the dRT is flawlessly continued (3′ to 5′ direction) into the 3′ overhang substrate sequence (framed in orange) followed by the mutated PAM site (green shaded) and intron sequence downstream of exon20 as exactly constructed in the RT. The top sequence panel of FIG. 14 shows the paste at 5′ DSB ends (vertical black dash line) between genome and the edited DNA fragment. As shown, a DSB occurred exactly at the 5′ gRNA target site on endogenous genomic DNA and the 5′ overhang substrate sequence (framed in orange) flanking the DSB was completely retained. Toward 3′ direction at this inner cut site, the 5′ overhang substrate sequence is transited into hPGK promoter+puromycin expression cassette (shaded in blue) without the mutated PAM site-containing 55 bp intron sequence 5′ to the cassette plus 70 bp 5′ portion of the cassette, a deletion of the 125 bp. It is likely that the 5′ end of the edited fragment excised from dRT was undergone DSB end processing led to deletions of the 5′ end sequences of 100 bp 5′ overhang substrate sequence and an additional 125 bp to the 5′ of hPGK promoter before re-joining with genome at the 5′ DSB site. However, the deletion of 55 bp intron sequence and 70 bp 5′ portion of hPGK promoter, indicated as a black outlined triangle in the top sequence panel and a gray-shaded section in a map in the middle of FIG. 14, does not compromise the structure of the edited area containing wildtype exon20 as the single nucleotide duplication was eliminated from the exon resulting in a restoration of TCC codon for Serine at 1497 as shown in red frame in the middle sequence panel of FIG. 14. Moreover, the expression of puromycin resistant gene was not compromised either as the edited cells were resistance to puromycin. Shown in the middle sequence panel of FIG. 14 are partial coding sequence of puromycin resistant gene (in blue frame) and partial exon 20 sequence (in black frame) showing the restored codon TCC for serine (F14975) in the iCAP edited mutant MED13L allele.

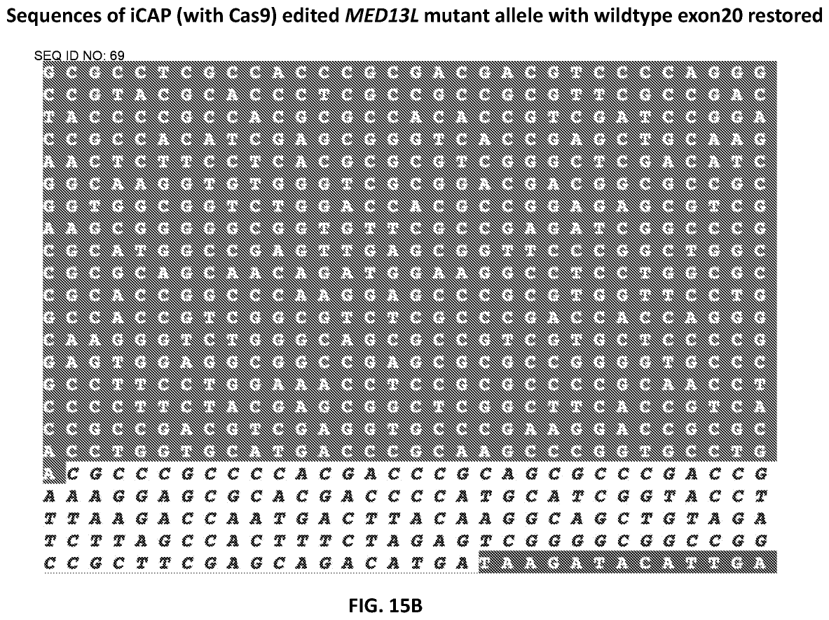

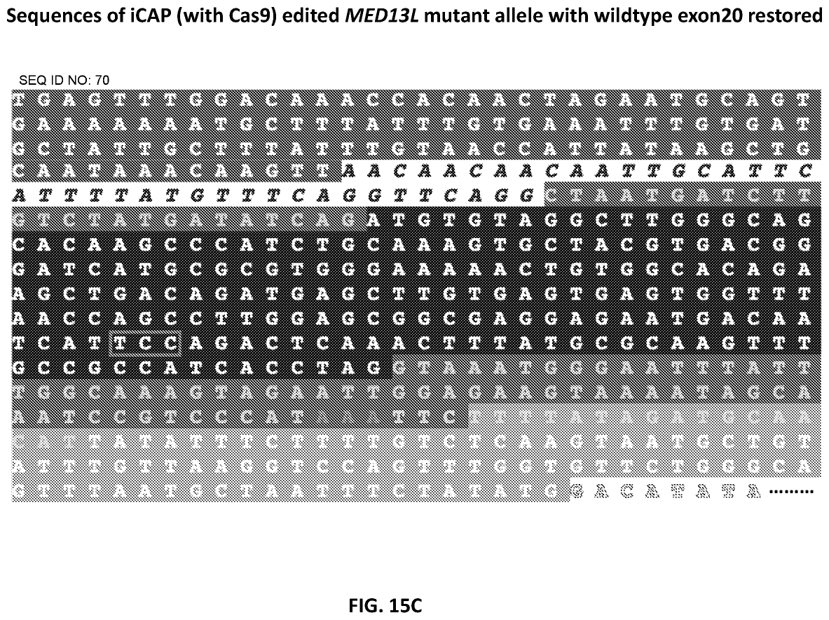

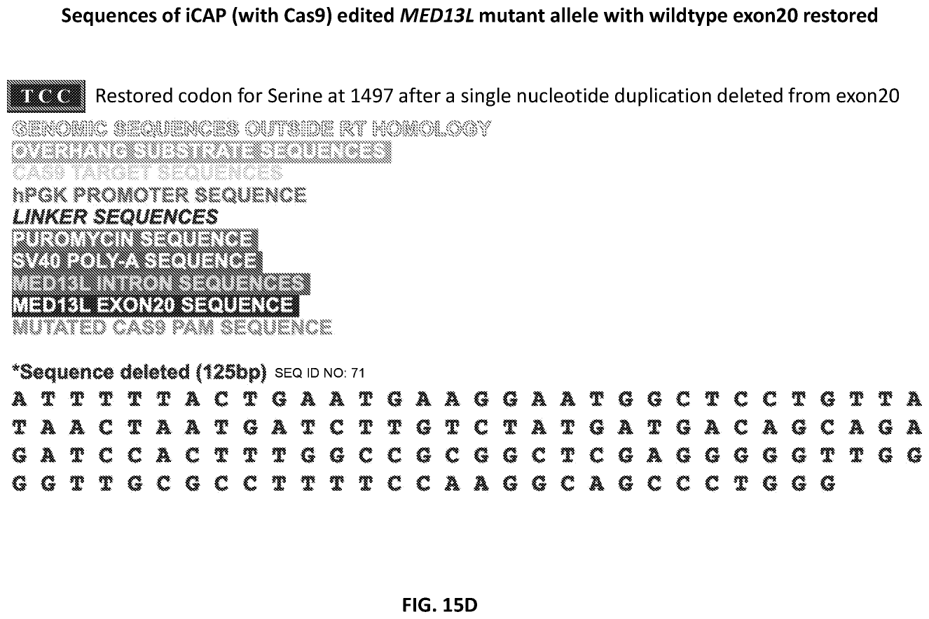

The sequences of the edited fragment pasted in the MED13L mutant allele and flanking endogenous genomic DNAs are illustrated in FIGS. 15A-15D, and the restored codon TCC without the single nucleotide Thymine duplication in exon20 are indicated in a red box in FIG. 15C. In total, these data illustrate the successful use of the iCAP process to precisely delete a single disease-causing nucleotide duplication in an exon of human genome to restore the reading frame for a functional MED13L protein production, further demonstrating the versatile utility of iCAP genome editing.

### Example 8: iCAP DNA Replacement Template (Donor) Design B for Deletion of a Single Disease-Causing Nucleotide Duplication from Exon20 of Human Mutant MED13L Allele in Patient Cells

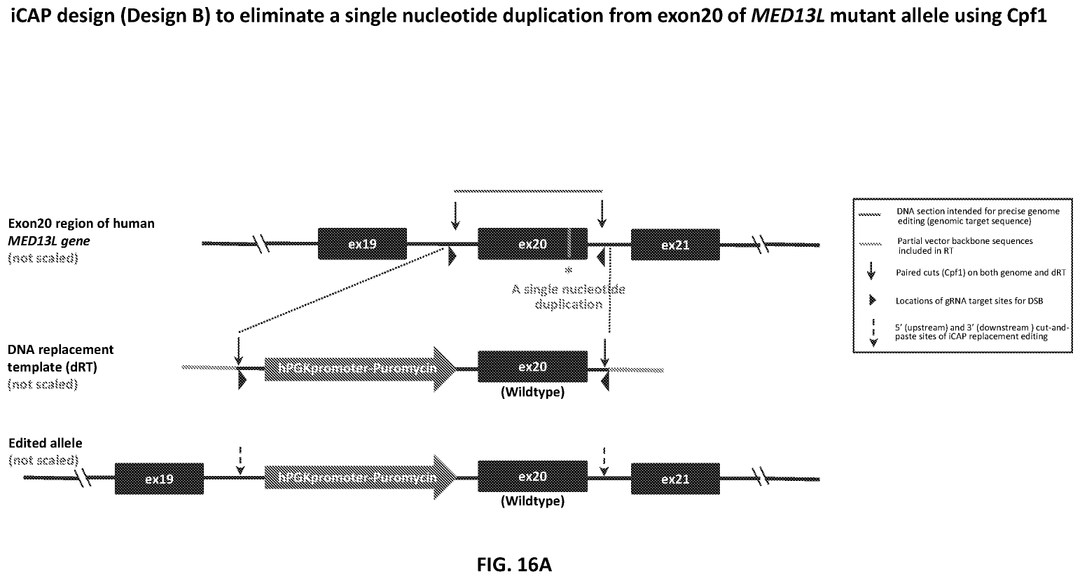

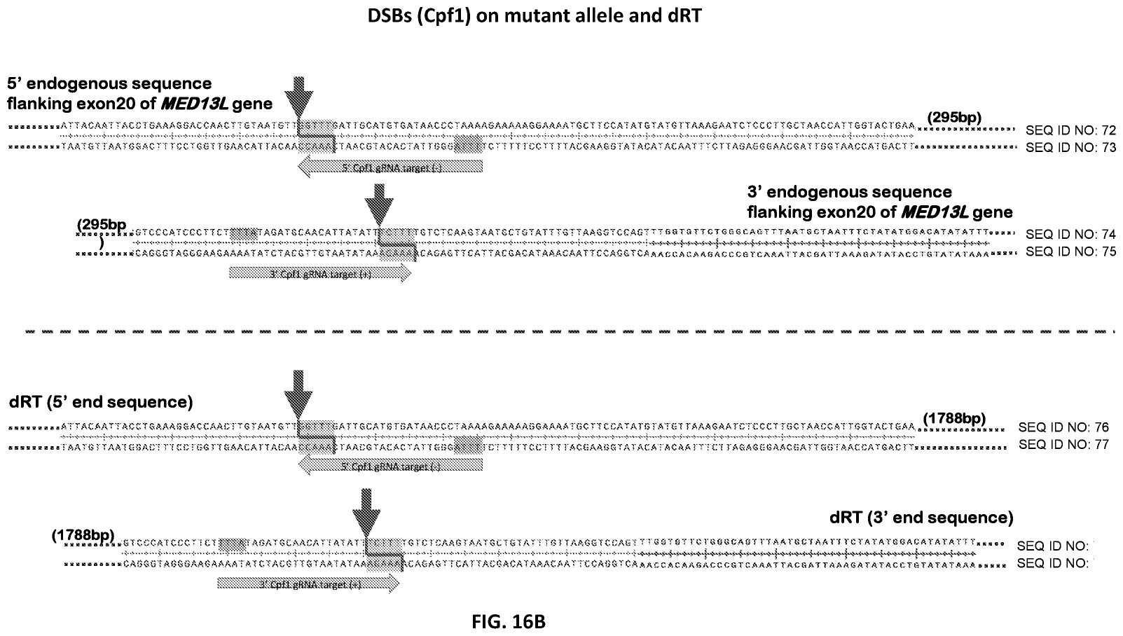

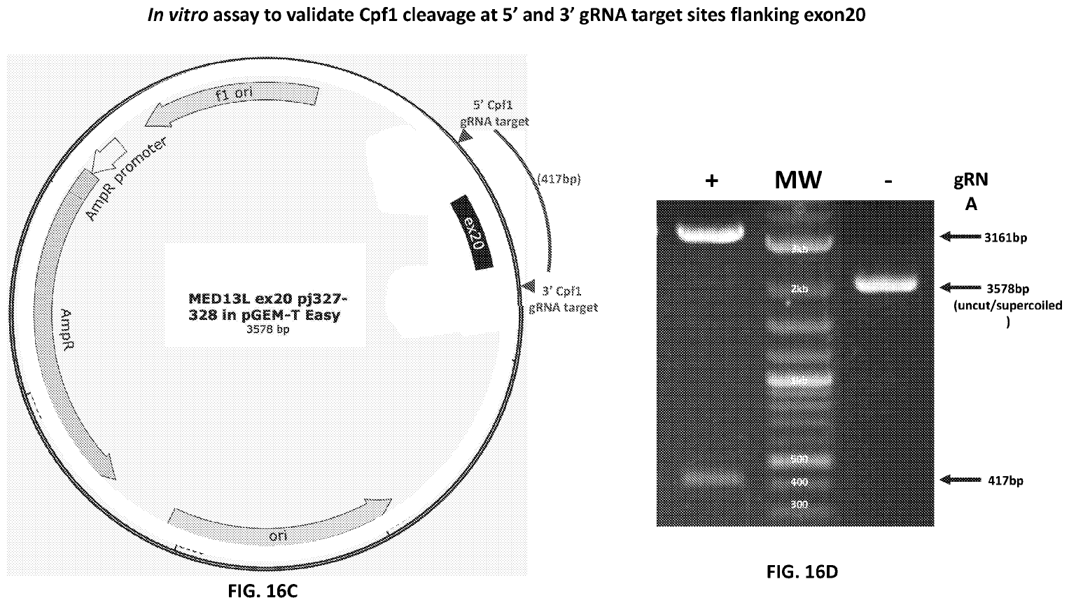

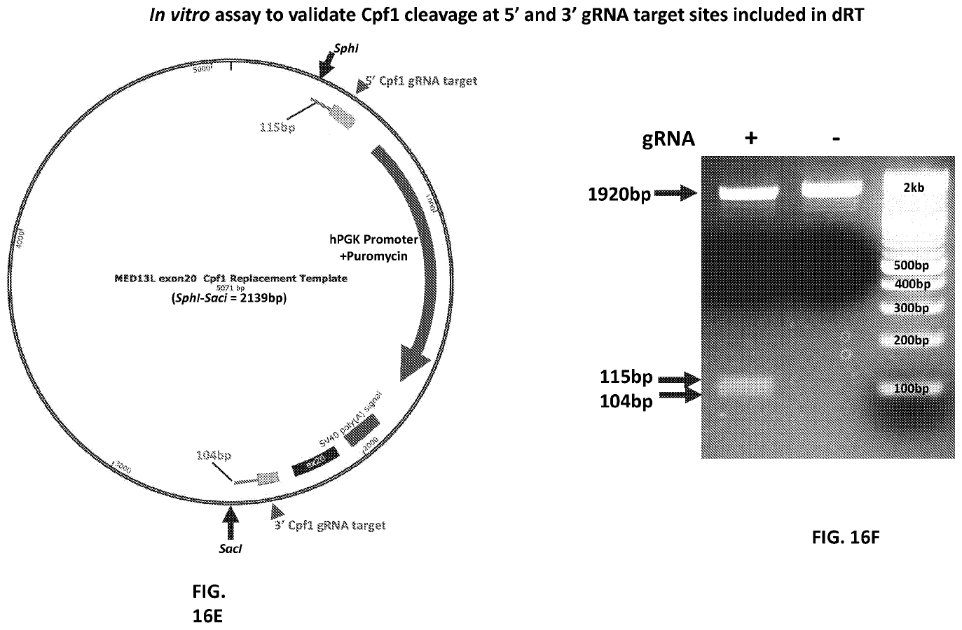

As an example of the use of the iCAP genomic editing Design B to precisely alter genome sequence at the level of individual nucleotides in human genome, a study was conducted to eliminate a single nucleotide duplication from exon20 of MED13L gene in genome of patient cells. As described in Example 6, the single base Thymine duplication in coding sequence (FIG. 12A) causes reading frame shift at Serine (S1497F) and consequently early termination of transcription, resulting in productions of truncated MED13L protein products with clinic manifestation of MED13L Syndrome. In this study, a dRT was constructed to contain (1) the edited fragment which was synthesized with the components, in the 5′ to 3′ direction, partial 5′ flanking intron sequence containing a 5′ gRNA target site and a selection marker puromycin resistant gene+wildtype exon20 (without the single T duplication)+partial 3′ flanking intron sequence containing a 3′ gRNA target site and (2) partial vector backbone sequences of 115 bp and 104 bp flanking 5′ and 3′ ends of the edited fragment (FIG. 16A, middle, and FIG. 16E). Importantly, there is no overhang substrate sequences (mini-homology sequences) flanking the edited fragment once excised from the dRT. Upon cleavages induced by Cas12a (Cpf1) in the study, DSB occurred at the same 5′ gRNA target site and the same 3′ gRNA target site presented on both genome and the dRT, and therefore no overlapping sequences were created at 5′ and 3′ DSB ends between genome and the edited fragment excised from the dRT (FIG. 16A-16B). The pastes of the edited fragment at 5′ and 3′ DSB ends on genome bridge (patch) the broken genome and replace the mutant-exon-containing intervening sequence with a wildtype exon20, resulting in the deletion of the disease-causing nucleotide duplication from the edited mutant allele as illustrated in FIG. 16A, bottom). An in vitro study was then conducted to verify the function of the gRNAs and Cpf1 nuclease. For production of the genomic target sequence, PCR was used to amplify a DNA fragment spanning the wildtype exon 20 of MED13L gene- and the PCR product was cloned in a vector and was then cleaved at gRNA target sites with sgRNAs and Cpf1 in vitro (FIG. 16C). Separation of the resulting reaction via agarose gel revealed DNA fragments of the expected sizes (FIG. 16D). A similar in vitro sgRNA/Cpf1 reaction was performed using the vector containing the dRT (FIG. 16E), and Cpf1 cleavage at gRNA target sites generated DNA fragments of the expected sizes (FIG. 16F). Together, these data indicated the successful design of the gRNAs and the dRT necessary for the iCAP genome editing as B Design.

### Example 9: Successful iCAP Editing (Design B) to Eliminate a Single Disease-Causing Nucleotide Duplication from Exon20 of Human Mutant MED13L Allele in Patient Cells

Patient cells of fibroblasts were transfected with a sgRNAs/Cas12a expression vector and dRT (Design B, FIGS. 16A and 16B) which is a 2139 bp SphI and SacI fragment (FIG. 16E) by electroporation followed by incubation in culture media for 48 hours and then in selection medium with puromycin. The surviving cell populations (labeled as 5-1-2) were harvested and genomic DNAs were extracted for genotypic analysis.

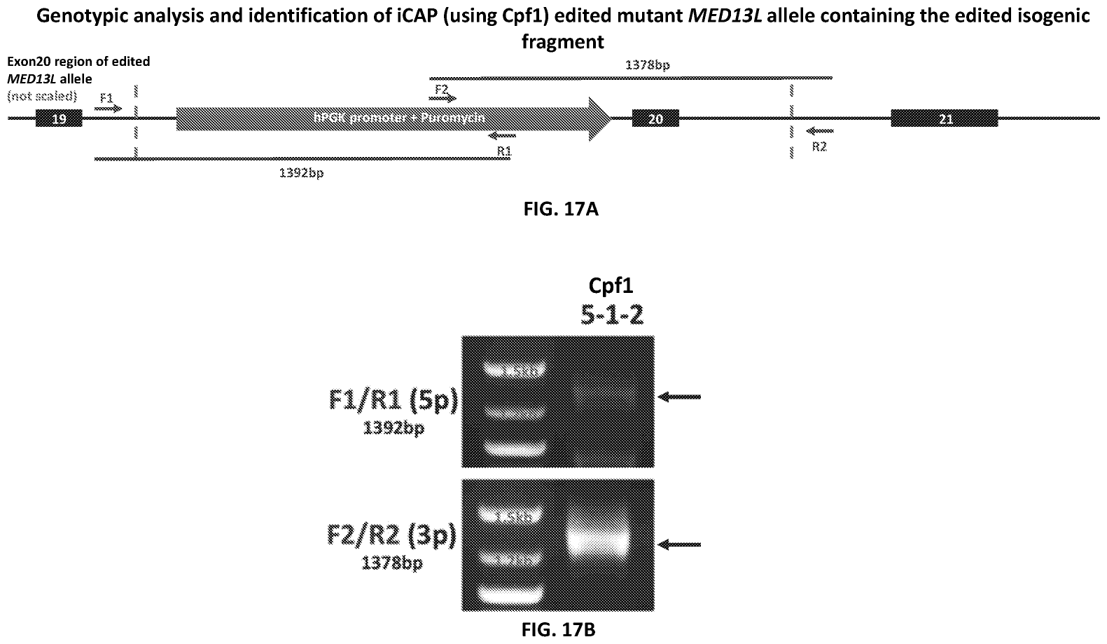

Shown in FIG. 17A is the position of primers (purple arrows) designed for identification of the new edited allele by PCR, and located both outside the range of the 5′ and 3′ gRNA target sites and within the new sequence of puromycin resistant gene. Using the primer pairs of F1-R1 and F2-R2, PCR products as predicted bands of about 1392 bp and 1378 bp were generated as indicated by horizontal black arrows in FIG. 17B. The PCR products span the 5′ (upstream) and 3′ (downstream) paste (re-joining) sites to puromycin resistant gene, respectively, suggesting the presence of the edited allele containing a puromycin resistant gene and the wild-type exon20, as a result of a replacement of the endogenous mutant-exon20-containing genomic DNA between two gRNA target sites with the edited fragment excised from the dRT. Vertical red dash lines in FIG. 17A indicate the 5′ and 3′ DSB sites on endogenous sequences as well as the limits of 5′ and 3′ end of the edited fragment, and horizontal purple arrows represent forward (F) and reverse (R) primers.

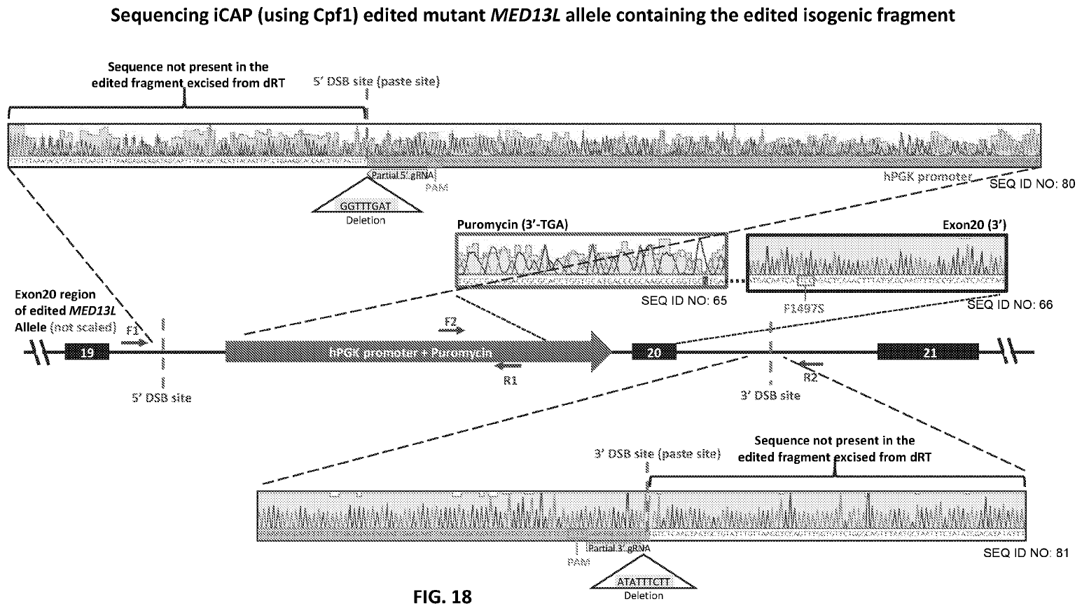

The PCR products were further analyzed by DNA sequencing to confirm a successfully iCAP editing of the mutant MED13L allele with the elimination of the single nucleotide duplication by the replacement of mutant exon20 as designed. As shown in the bottom sequence panel of FIG. 18, the paste at 3′ DSB ends (vertically red dash line) between genome and the edited DNA fragment is near seamless with only 9 bp deletion occurred within the 3′ gRNA target site sequence (for Cpf1 recognition and cleavage). It appears that the small deletion at the 3′ DSB ends of both genome and the edited fragment resulted in conversions of the 5′ overhang ends created by Cpf1 cleavage at the DSBs into blunt ends before the ends were re-joined. Such a minimal change within gRNA target site sequence also appears to be beneficial as the original gRNA target sequence was disrupted at the re-joining site due to the deletion. As a DSB creates two broken ends, a 5′ end and a 3′ end, the 5′ overhang at the 3′ end of the 3′ DSB induced by Cpf1 on genome is complementary to the 5′ overhang at the 5′ end of the 3′ DSB induced by Cpf1 on dRT (resulting in the edited fragment's 3′ end released from dRT) because of the cleavages occurred at the exactly same gRNA target sequence. The complementary cohesive 5′ overhang ends would re-join the edited fragment with genome at 3′ DSB site and the same gRNA target sequence would consequently be reconstituted if modifications did not occur at the 5′ overhang ends, rendering the re-joining site to be cleaved again. The top sequence panel of FIG. 18 shows the paste at 5′ DSB ends (vertically arrowed red dash line) between genome and the edited DNA fragment. Similar to the 3′ paste site, the 5′ paste site is also near flawless with only 8 bp deleted, again within the 5′ gRNA target site sequence (for Cpf1 recognition and cleavage). The small deletion also converted the 5′ overhang ends created by Cpf1 cleavage at the 5′ DSB sites of both genome and the edited fragment into blunt ends for re-joining of genome and the edited fragment for the same beneficial effect as described for the 3′ paste site.

As the gRNA target sites are intentionally screened and selected in the intron sequences for the reason that is no significant impact on gene functions if certain changes occurred in the introns, less than 10 bp deletions observed here therefore did not compromise the structure of the edited area containing wildtype exon20 as the single nucleotide duplication was eliminated from the exon resulting in a restoration of TCC codon for Serine (F1497S) as shown in red frame in the middle sequence panel of FIG. 18. In addition, the expression of puromycin resistant gene was not compromised either as the edited cells were resistance to puromycin. The results indicated that the minimal changes at paste sites in intron sequences did not have any impact on gene functions. Shown in the middle sequence panel of FIG. 18 are partial coding sequence of puromycin resistant gene and partial exon 20 sequence showing the restored codon TCC for serine (F14975) in the edited mutant MED13L allele.

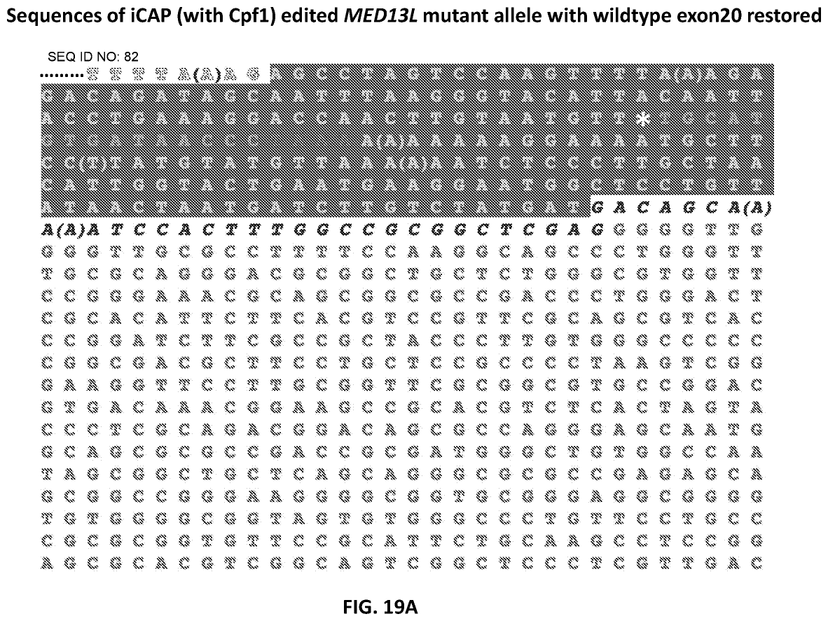

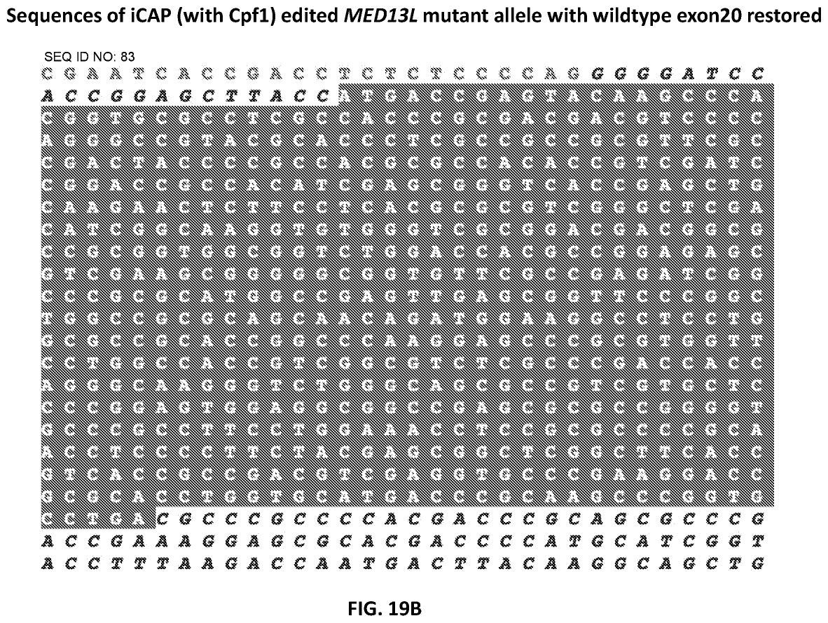

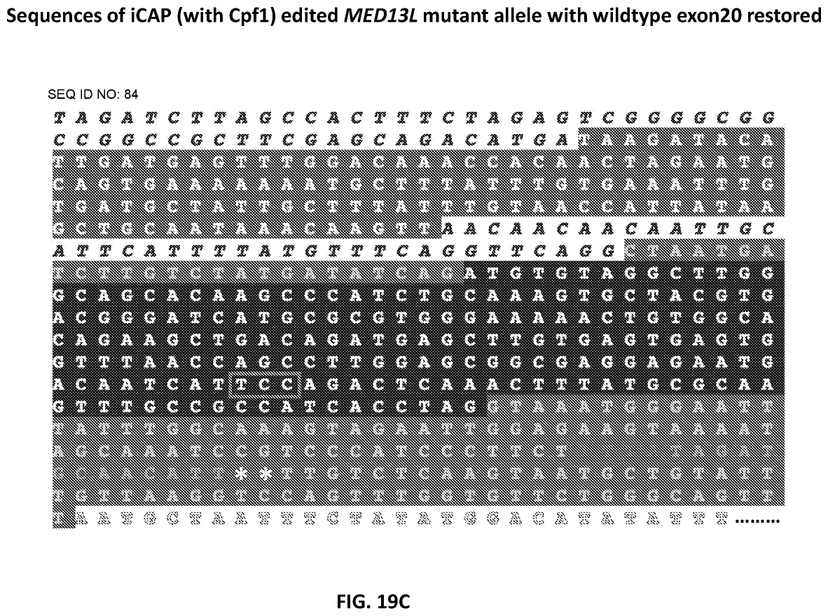

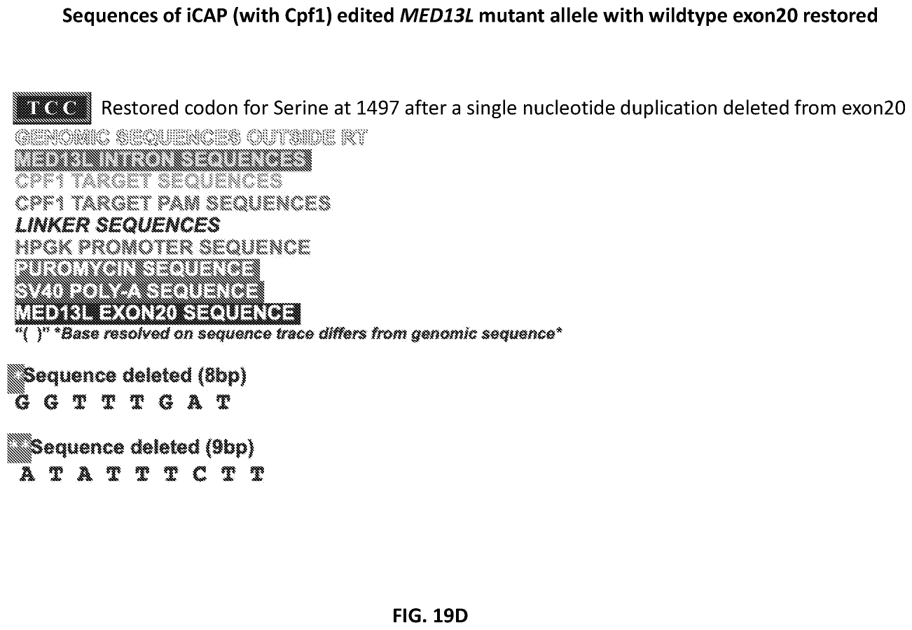

The sequences of the edited fragment pasted in the MED13L mutant allele and flanking endogenous genomic DNAs are illustrated in FIGS. 19A-19D, and the restored codon TCC without the single nucleotide Thymine duplication in exon20 are indicated in a red box in FIG. 19C.

In total, these data illustrate the successful use of the iCAP process through usage of Cpf1 programmable nuclease to precisely delete a single disease-causing nucleotide duplication in an exon of human genome to restore codon and the reading-frame, further demonstrating the versatile utility of iCAP genome editing and the flexibility of iCAP in using different Cas programmable nucleases and thereby enabling iCAP with significantly increased availabilities and choices of gRNA target sites to edit complex genome. In addition, the data also indicate that through flexible design choices iCAP genome editing process could mobilize the appropriate DNA damage repair pathways, if not all, to facilitate end re-joining of a broken genome at two designed DSB sites with an edited isogenic fragment which can be pre-constructed to contain altered nucleotide sequence compositions and replaces the excised endogenous intervening sequence between the two DSBs.

### Example 10: Successfully Perfect Deletion of a Section of Endogenous Sequences Between DSBs and Flawless Re-Joining of the Broken Genome without the Intervening Sequences by iCAP

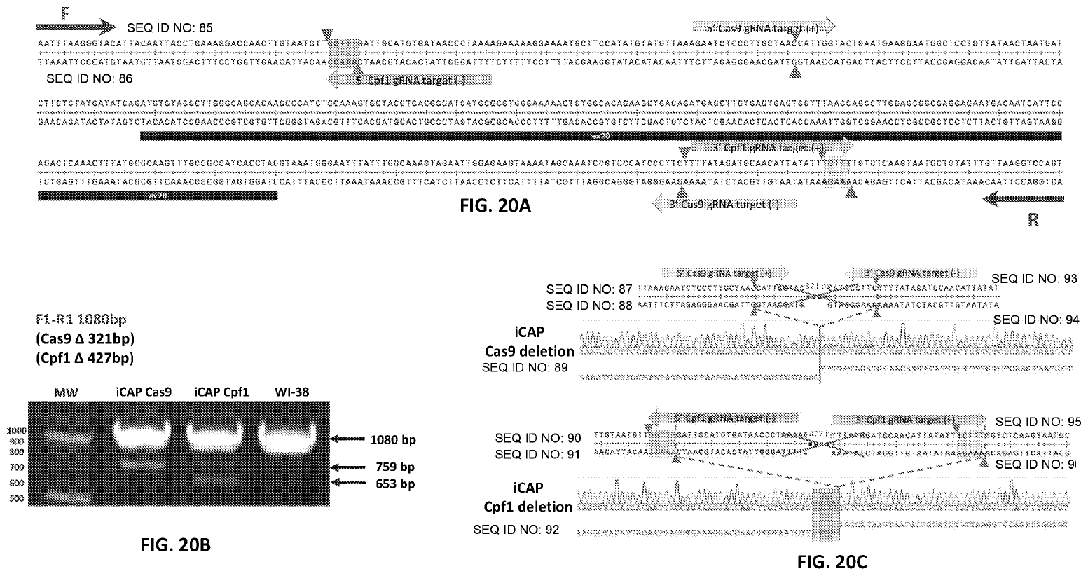

The iCAP genome editing also demonstrated that it enables a precise deletion of a section of endogenous genome sequences between two gRNA target sites cleaved by programmable nucleases such as Cas9 and Cpf1 (Cas12a) and results in a flawless end re-joining of broken genome with or without the presence of dRT. In a study, gRNA target sites recognized by either Cas9 or Cpf1 were identified in introns on either sides of exon20 of human MED13L gene as shown in FIG. 20A. To valid the effectiveness of these gRNA target sites in inducing DSBs in vivo, MED13L Syndrome patient cells of fibroblasts were transfected with either a sgRNAs/Cas9 expression vector (labeled as iCAP Cas9) or a sgRNAs/Cpf1 expression vector (labeled as iCAP Cpf1) by electroporation followed by incubation in culture medium for 24 hours. The transfected cells were then collected and genomic DNAs were extracted for genotypic analysis. Using primers as indicated with horizontal purple solid arrow in FIG. 20A, PCR products were amplified from genomic DNAs extracted from different groups of transfected cell population. The presence of PCR products with expected sizes, a 1080 bp band with no cleavage and no sequence deleted, a 759 bp band with Cas9 cleavages and a deletion of 321 bp and a 653 bp band with Cpf1 cleavages and a deletion of 427 bp as shown in the agarose gel of FIG. 20B, indicated deletions of intervening sequences between two gRNA target sites in the MED13L gene. To further evaluate the structure of the re-joined ends of the genome without the intervening sequence of exon20 between the two DSB sites, DNA sequencing analysis of the PCR products was conducted. As shown in FIG. 20A and the top sequence panel of FIG. 20C, Cas9 cleavages generated blunt ends at both 5′ (upstream) and 3′ (downstream) gRNA target sites, and the 5′ end of the upstream DSB site and the 3′ end of the downstream DSB site of broken genome were flawlessly re-joined without the intervening sequence, indicating no any modification at the blunt ends (top sequence panel of FIG. 20C, a red vertical line indicates where the re-joining occurred). Also shown in FIG. 20A and the bottom sequence panel of FIG. 20C, Cpf1 cleavages generated 5′ overhang ends at both 5′ (upstream) and 3′ (downstream) gRNA target sites. However, the 5′ cohesive end at the upstream DSB site and the 5′ cohesive end at the downstream DSB site are not compatible for annealing to re-join the broken genome ends without the intervening sequence containing exon20, due to differences of the two gRNA target sequences. As shown in the bottom panel of FIG. 20C, it appears that both of the cohesive ends created by Cpf1 cleavage in cells were converted to blunt ends with the 5′ overhang of CCAAA filled-in with pairing nucleotides GGTTT at the 5′ DSB end (shaded in light brown) while the 5′ overhang of TCTTT deleted at the 3′ DSB end (shaded in gray), resulting in a flawless re-joining of the two broken genome ends with the intervening endogenous sequence deleted (a red vertical line in the bottom sequence panel of FIG. 20C indicates where the re-joining occurred). The sequencing trace from the pooled cell populations also suggests the existance of another sequence species in which the formats of blunt end conversions at the 5′ and 3′ DSB sites are a reversal of what was described as above. The re-joining of two DSB ends created by either Cas9 or Cpf1 with overhang ends blunted abolished both of the 5′ and 3′ gRNA target sites to avoid further cleavage making the flawless linkage “permanent”. In total, these data illustrate the successful use of the iCAP process to precisely delete a section of endogenous sequence from genome in a simplest way, further demonstrating that the single iCAP genome editing platform has multiple utilities and is able to precisely alter nucleotide sequence composition in a variety of types such as single base alterations, exon deletion or replacement, precise insertions of exogenous sequences, precise deletions of endogenous sequences, etc., for the mammalian genomes.

### Other Embodiments

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of editing, mutating, or modifying a genomic target DNA sequence in a cell, the method comprising:
a. providing a DNA replacement template (RT) comprising
(i) the target DNA sequence comprising the desired edited, mutated, and/or modified nucleotide(s), and
(ii) a sequence encoding a nuclease,
b. contacting the genomic target DNA sequence, the DNA RT, and heterologous guide-RNAs (gRNAs) under conditions that allow for the gRNAs to induce double-strand breaks of the genomic target DNA sequence and the DNA RT by the nuclease generating either blunt ends or overhanging ends,
c. subjecting the blunt ends of the genomic target DNA sequence and DNA RT to 5′ to 3′ DNA end resection to generate complementary 3′ overhangs,
d. annealing complementary 3′ overhangs of the DNA replacement template to the complementary 3′ overhang sequences flanking the genomic target DNA sequence, and
e. ligating the annealing sites, thereby resulting in incorporation of the DNA RT into the place of the genomic target DNA sequence.

2. The method of claim 1, further comprising:
f. subjecting the overhanging ends flanking the endogenous genomic target DNA sequence and the DNA RT to modification resulting in blunt ends, and
g. ligating the blunt ends of RT to the genomic target DNA sequence, thereby resulting in incorporation of the DNA RT into the place of the genomic target DNA sequence.

3. The method of claim 1, wherein the blunt ends generated by the nuclease or resulting from modification of the overhanging ends of the target DNA sequence are ligated together, thereby resulting in the deletion of the target DNA sequence.

4. The method of claim 1, wherein the nuclease is a Cas9 nuclease.

5. The method of claim 4, wherein the Cas9 nuclease is a naturally-occurring variant thereof.

6. The method of claim 5, wherein the naturally-occurring Cas9 nuclease variant comprises at least one of SpCas9, SaCas9, StCas9, NmCas9, FnCas9, CjCas9, CasX, CasY, Cas12a, Cas14a, BlCas9, ScCas9, LmoCas9, TdCas9, Nme2Cas9, GsCas9, BlatCas9, and FnCas9-RHA.

7. The method of claim 6, wherein the naturally-occurring Cas12a nuclease variant comprises at least one of AsCpf1, FnCpf1, LbCpf1, AsCpf1-RR, LbCpf1-RR, and AsCpf1-RVR.

8. The method of claim 1, wherein the nuclease is a Cas variant nuclease.

9. The method of claim 8, wherein the Cas variant nuclease comprises at least one of Cas13a/b(C2c2), Cas12b(C2c1), and Cas12c(C2c3).

10. The method of claim 1, wherein the Cas9 nuclease is an engineered variant thereof.

11. The method of claim 10, wherein the engineered Cas9 nuclease variant comprises at least one of eSpCas9, SpCas9-HF1, Fok1-Fused dCas9, xCas9, SpCas9-VQR, SpCas9-VRER, SpCas9-D1135E, SpCas9-EQR, SpCas9-QQR1, Cas9-DD, HypaCas9, evoCas9, xCas9-3.7, SniperCas9, Cas9-CtIP, SpCas9-NG, Split-SpCas9, SpCas9-K855A, ScCas9+, ScCas9++, SaCas9-KKH, and SaCas9.

12. The method of claim 1, wherein the cell is a eukaryotic cell.

13. The method of claim 12, wherein the eukaryotic cell is a mammalian cell.

14. The method of claim 13, wherein the mammalian cell is part of a tissue or organism and the method is performed in situ.

15. The method of claim 13, wherein the mammalian cell is a developing embryo.
